SEQUENCE LISTING (140 sequences)

SEQ ID NO: 1 - protein, 535 aa, synthetic construct
QPPPPPPDATCHQVRSFFQRLQPGLKWVPETPVPGSDLQVCLPKGPTCCSRKMEEKYQLTARLNMEQLLQSASMELKFLIIQNAAVFQEAFEIVVRHAKNYTNAMFKNNYPSLTPQAFEFVGEFFTDVSLYILGSDINVDDMVNELFDSLFPVIYTQLMNPGLPDSALDINECLRGARRDLKVFGNFPKLIMTQVSKSLQVTRIFLQALNLGIEVINTTDHLKFSKDCGRMLTRMWYCSYCQGLMMVKPCGGYCNVVMQGCMAGVVEIDKYWREYILSLEELVNGMYRIYDMENVLLGLFSTIHDSIQYVQKNAGKLTTTIGKLCAHSQQRQYRSAYYPEDLFIDKKVLKVAHVEHEETLSSRRRELIQKLKSFISFYSALPGYICSHSPVAENDTLCWNGQELVERYSQKAARNGMKNQFNLHELKMKGPEPVVSQIIDKLKHINQLLRTMSMPKGRVLDKNLDEEGFESGDCGDDEDECIGGSGDGMIKVKNQLRFLAELAYDLDVDDAPGNSQQATPKDNEISTFHNLGNVH

SEQ ID NO: 2 - protein, 242 aa, synthetic construct
DVVMTQTPLSLPVSLGDQASISCRSSQSLVHSNGNTYLHWYLQKPGQSPKLLIYKVSNRFSGVPDRFSGSGSGTDFTLKISRVEAEDLGVYFCSQNTHVPPTFGSGTKLEIKGGGGSGGGGSGGGGSQVQLQQSGAELVRPGASVKLSCKASGYTFTDYEMHWVKQTPVHGLKWIGALDPKTGDTAYSQKFKGKATLTADKSSSTAYMELRSLTSEDSAVYYCTRFYSYTYWGQGTLVTVSA

SEQ ID NO: 3 - protein, 115 aa, synthetic construct
QVQLVQSGAEVKKPGASVKVSCKASGYTFTDYEMHWVRQAPGQGLEWMGALDPKTGDTAYSQKFKGRVTMTRDTSTSTVYMELSSLRSEDTAVYYCARFYSYTYWGQGTLVTVSS

SEQ ID NO: 4 - protein, 112 aa, synthetic construct
DIVMTQTPLSLSVTPGQPASISCRSSQSLVHSNGNTYLHWYLQKPGQSPQLLIYKVSNRFSGVPDRFSGSGSGTDFTLKISRVEAEDVGVYYCSQNTHVPPTFGQGTKVEIK

SEQ ID NO: 5 - protein, 115 aa, synthetic construct
QVQLVQSGAEVKKPGASVKVSCKASGYTFTDYEMHWVRQAPGQGLEWMGALDPKTGDTAYSQKFKGRVTLTADTSTSTVYMELSSLRSEDTAVYYCTRFYSYTYWGQGTLVTVSS

SEQ ID NO: 6 - protein, 115 aa, synthetic construct
QVQLVQSGAEVKKPGASVKVSCKASGYTFTSYEMHWVRQAPGQGLEWMGALDPKTGDTAYSQKFKGRVTLTADTSTSTVYMELSSLRSEDTAVYYCTRFYSYTYWGQGTLVTVSS

SEQ ID NO: 7 - protein, 115 aa, synthetic construct
QVQLVQSGAEVKKPGASVKVSCKASGYTFTDYEMHWVRQAPGQGLEWMGALDPKTGDTAYSQKFQGRVTLTADTSTSTVYMELSSLRSEDTAVYYCTRFYSYTYWGQGTLVTVSS

SEQ ID NO: 8 - protein, 115 aa, synthetic construct
QVQLVQSGAEVKKPGASVKVSCKASGYTFTSYEMHWVRQAPGQGLEWMGALDPKTGDTAYSQKFQGRVTLTADTSTSTVYMELSSLRSEDTAVYYCTRFYSYTYWGQGTLVTVSS

SEQ ID NO: 9 - protein, 112 aa, synthetic construct
DIVMTQTPLSLSVTPGQPASISCRSSQSLVHSNGNTYLHWYLQKPGQSPQLLIYKVSSRFSGVPDRFSGSGSGTDFTLKISRVEAEDVGVYYCSQNTHVPPTFGQGTKVEIK

SEQ ID NO: 10 - protein, 15 aa, synthetic construct
GGGGSGGGGSGGGGS

SEQ ID NO: 11 - protein, 115 aa, synthetic construct
QVQLVQSGAEVKKPGASVKVSCKASGYTFTSYEMHWVRQAPGQGLEWMGALDPSTGDTAYSQKFQGRVTLTADTSTSTVYMELSSLRSEDTAVYYCTRFYSYTYWGQGTLVTVSS

SEQ ID NO: 12 - protein, 115 aa, synthetic construct
QVQLVQSGAEVKKPGASVKVSCKASGYTFTSYEMHWVRQAPGQGLEWMGALDPKGGDTAYSQKFQGRVTLTADTSTSTVYMELSSLRSEDTAVYYCTRFYSYTYWGQGTLVTVSS

SEQ ID NO: 13 - protein, 115 aa, synthetic construct
QVQLVQSGAEVKKPGASVKVSCKASGYTFTSYEMHWVRQAPGQGLEWMGALDPSGGDTAYSQKFQGRVTLTADTSTSTVYMELSSLRSEDTAVYYCTRFYSYTYWGQGTLVTVSS

SEQ ID NO: 14 - protein, 115 aa, synthetic construct
QVQLVQSGAEVKKPGASVKVSCKASGYTFTSYEMHWVRQAPGQGLEWMGALDPKTGSTAYSQKFQGRVTLTADTSTSTVYMELSSLRSEDTAVYYCTRFYSYTYWGQGTLVTVSS

SEQ ID NO: 15 - protein, 115 aa, synthetic construct
QVQLVQSGAEVKKPGASVKVSCKASGYTFTSYEMHWVRQAPGQGLEWMGALDPSGGSTAYSQKFQGRVTLTADTSTSTVYMELSSLRSEDTAVYYCTRFYSYTYWGQGTLVTVSS

SEQ ID NO: 16 - protein, 112 aa, synthetic construct
DIVMTQTPLSLSVTPGQPASISCRSSQSLVHSNGKTYLHWYLQKPGQSPQLLIYKVSSRFSGVPDRFSGSGSGTDFTLKISRVEAEDVGVYYCSQNTHVPPTFGQGTKVEIK

SEQ ID NO: 17 - protein, 112 aa, synthetic construct
DIVMTQTPLSLSVTPGQPASISCRSSQSLVHSNGNTYLHWYLQKPGQSPQLLIYKVSSRFSGVPDRFSGSGSGTDFTLKISRVEAEDVGVYYCMQNTHVPPTFGQGTKVEIK

SEQ ID NO: 18 - protein, 112 aa, synthetic construct
DIVMTQTPLSLSVTPGQPASISCRSSQSLVHSNGNTYLHWYLQKPGQSPQLLIYKVSSRFSGVPDRFSGSGSGTDFTLKISRVEAEDVGVYYCSQGTHVPPTFGQGTKVEIK

SEQ ID NO: 19 - protein, 112 aa, synthetic construct
DIVMTQTPLSLSVTPGQPASISCRSSQSLVHSNGNTYLHWYLQKPGQSPQLLIYKVSSRFSGVPDRFSGSGSGTDFTLKISRVEAEDVGVYYCSQNTHWPPTFGQGTKVEIK

SEQ ID NO: 20 - protein, 10 aa, synthetic construct
GYTFTDYEMH

SEQ ID NO: 21 - protein, 10 aa, synthetic construct
GYTFTSYEMH

SEQ ID NO: 22 - protein, 17 aa, synthetic construct
ALDPKTGDTAYSQKFKG

SEQ ID NO: 23 - protein, 17 aa, synthetic construct
ALDPKTGDTAYSQKFQG

SEQ ID NO: 24 - protein, 17 aa, synthetic construct
ALDPSTGDTAYSQKFQG

SEQ ID NO: 25 - protein, 17 aa, synthetic construct
ALDPKGGDTAYSQKFQG

SEQ ID NO: 26 - protein, 17 aa, synthetic construct
ALDPSGGDTAYSQKFQG

SEQ ID NO: 27 - protein, 17 aa, synthetic construct
ALDPKTGSTAYSQKFQG

SEQ ID NO: 28 - protein, 17 aa, synthetic construct
ALDPSGGSTAYSQKFQG

SEQ ID NO: 29 - protein, 6 aa, synthetic construct
FYSYTY

SEQ ID NO: 30 - protein, 16 aa, synthetic construct
RSSQSLVHSNGNTYLH

SEQ ID NO: 31 - protein, 16 aa, synthetic construct
RSSQSLVHSNGKTYLH

SEQ ID NO: 32 - protein, 7 aa, synthetic construct
KVSNRFS

SEQ ID NO: 33 - protein, 7 aa, synthetic construct
KVSSRFS

SEQ ID NO: 34 - protein, 9 aa, synthetic construct
SQNTHVPPT

SEQ ID NO: 35 - protein, 9 aa, synthetic construct
MQNTHVPPT

SEQ ID NO: 36 - protein, 9 aa, synthetic construct
SQGTHVPPT

SEQ ID NO: 37 - protein, 9 aa, synthetic construct
SQNTHWPPT

SEQ ID NO: 38 - protein, 242 aa, synthetic construct
DVVMTQSPLSLPVTPGEPASISCRSSQSLVHSNGNTYLHWYLQKPGQSPQLLIYKVSNRFSGVPDRFSGSGSGTDFTLKISRVEAEDVGVYYCSQNTHVPPTFGQGTKLEIKGGGGSGGGGSGGGGSQVQLVQSGAEVKKPGASVKVSCKASGYTFTDYEMHWVRQAPGQGLEWMGALDPKTGDTAYSQKFKGRVTLTADKSTSTAYMELSSLTSEDTAVYYCTRFYSYTYWGQGTLVTVSS

SEQ ID NO: 39 - protein, 242 aa, synthetic construct
DIVMTQTPLSLSVTPGQPASISCRSSQSLVHSNGNTYLHWYLQKPGQSPQLLIYKVSNRFSGVPDRFSGSGSGTDFTLKISRVEAEDVGVYYCSQNTHVPPTFGQGTKVEIKGGGGSGGGGSGGGGSQVQLVQSGAEVKKPGASVKVSCKASGYTFTDYEMHWVRQAPGQGLEWMGALDPKTGDTAYSQKFKGRVTLTADTSTSTVYMELSSLRSEDTAVYYCTRFYSYTYWGQGTLVTVSS

SEQ ID NO: 40 - protein, 242 aa, synthetic construct
DIVMTQTPLSLSVTPGQPASISCRSSQSLVHSNGNTYLHWYLQKPGQSPQLLIYKVSNRFSGVPDRFSGSGSGTDFTLKISRVEAEDVGVYYCSQNTHVPPTFGQGTKVEIKGGGGSGGGGSGGGGSQVQLVQSGAEVKKPGASVKVSCKASGYTFTSYEMHWVRQAPGQGLEWMGALDPKTGDTAYSQKFKGRVTLTADTSTSTVYMELSSLRSEDTAVYYCTRFYSYTYWGQGTLVTVSS

SEQ ID NO: 41 - protein, 242 aa, synthetic construct
DIVMTQTPLSLSVTPGQPASISCRSSQSLVHSNGNTYLHWYLQKPGQSPQLLIYKVSNRFSGVPDRFSGSGSGTDFTLKISRVEAEDVGVYYCSQNTHVPPTFGQGTKVEIKGGGGSGGGGSGGGGSQVQLVQSGAEVKKPGASVKVSCKASGYTFTDYEMHWVRQAPGQGLEWMGALDPKTGDTAYSQKFQGRVTLTADTSTSTVYMELSSLRSEDTAVYYCTRFYSYTYWGQGTLVTVSS

SEQ ID NO: 42 - protein, 242 aa, synthetic construct
DIVMTQTPLSLSVTPGQPASISCRSSQSLVHSNGNTYLHWYLQKPGQSPQLLIYKVSNRFSGVPDRFSGSGSGTDFTLKISRVEAEDVGVYYCSQNTHVPPTFGQGTKVEIKGGGGSGGGGSGGGGSQVQLVQSGAEVKKPGASVKVSCKASGYTFTSYEMHWVRQAPGQGLEWMGALDPKTGDTAYSQKFQGRVTLTADTSTSTVYMELSSLRSEDTAVYYCTRFYSYTYWGQGTLVTVSS

SEQ ID NO: 43 - protein, 242 aa, synthetic construct
DIVMTQTPLSLSVTPGQPASISCRSSQSLVHSNGNTYLHWYLQKPGQSPQLLIYKVSSRFSGVPDRFSGSGSGTDFTLKISRVEAEDVGVYYCSQNTHVPPTFGQGTKVEIKGGGGSGGGGSGGGGSQVQLVQSGAEVKKPGASVKVSCKASGYTFTDYEMHWVRQAPGQGLEWMGALDPKTGDTAYSQKFKGRVTLTADTSTSTVYMELSSLRSEDTAVYYCTRFYSYTYWGQGTLVTVSS

SEQ ID NO: 44 - protein, 242 aa, synthetic construct
DIVMTQTPLSLSVTPGQPASISCRSSQSLVHSNGNTYLHWYLQKPGQSPQLLIYKVSSRFSGVPDRFSGSGSGTDFTLKISRVEAEDVGVYYCSQNTHVPPTFGQGTKVEIKGGGGSGGGGSGGGGSQVQLVQSGAEVKKPGASVKVSCKASGYTFTSYEMHWVRQAPGQGLEWMGALDPKTGDTAYSQKFKGRVTLTADTSTSTVYMELSSLRSEDTAVYYCTRFYSYTYWGQGTLVTVSS

SEQ ID NO: 45 - protein, 242 aa, synthetic construct
DIVMTQTPLSLSVTPGQPASISCRSSQSLVHSNGNTYLHWYLQKPGQSPQLLIYKVSSRFSGVPDRFSGSGSGTDFTLKISRVEAEDVGVYYCSQNTHVPPTFGQGTKVEIKGGGGSGGGGSGGGGSQVQLVQSGAEVKKPGASVKVSCKASGYTFTDYEMHWVRQAPGQGLEWMGALDPKTGDTAYSQKFQGRVTLTADTSTSTVYMELSSLRSEDTAVYYCTRFYSYTYWGQGTLVTVSS

SEQ ID NO: 46 - protein, 242 aa, synthetic construct
DIVMTQTPLSLSVTPGQPASISCRSSQSLVHSNGNTYLHWYLQKPGQSPQLLIYKVSSRFSGVPDRFSGSGSGTDFTLKISRVEAEDVGVYYCSQNTHVPPTFGQGTKVEIKGGGGSGGGGSGGGGSQVQLVQSGAEVKKPGASVKVSCKASGYTFTSYEMHWVRQAPGQGLEWMGALDPKTGDTAYSQKFQGRVTLTADTSTSTVYMELSSLRSEDTAVYYCTRFYSYTYWGQGTLVTVSS

SEQ ID NO: 47 - protein, 21 aa, synthetic construct
MALPVTALLLPLALLLHAARP

SEQ ID NO: 48 - protein, 8 aa, synthetic construct
DYKDDDDK

SEQ ID NO: 49 - protein, 45 aa, synthetic construct
TTTPAPRPPTPAPTIASQPLSLRPEACRPAAGGAVHTRGLDFACD

SEQ ID NO: 50 - protein, 24 aa, synthetic construct
IYIWAPLAGTCGVLLLSLVITLYC

SEQ ID NO: 51 - protein, 42 aa, synthetic construct
KRGRKKLLYIFKQPFMRPVQTTQEEDGCSCRFPEEEEGGCEL

SEQ ID NO: 52 - protein, 112 aa, synthetic construct
RVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 53 - protein, 494 aa, synthetic construct
MALPVTALLLPLALLLHAARPDYKDDDDKDVVMTQSPLSLPVTPGEPASISCRSSQSLVHSNGNTYLHWYLQKPGQSPQLLIYKVSNRFSGVPDRFSGSGSGTDFTLKISRVEAEDVGVYYCSQNTHVPPTFGQGTKLEIKGGGGSGGGGSGGGGSQVQLVQSGAEVKKPGASVKVSCKASGYTFTDYEMHWVRQAPGQGLEWMGALDPKTGDTAYSQKFKGRVTLTADKSTSTAYMELSSLTSEDTAVYYCTRFYSYTYWGQGTLVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 54 - protein, 494 aa, synthetic construct
MALPVTALLLPLALLLHAARPDYKDDDDKDIVMTQTPLSLSVTPGQPASISCRSSQSLVHSNGNTYLHWYLQKPGQSPQLLIYKVSNRFSGVPDRFSGSGSGTDFTLKISRVEAEDVGVYYCSQNTHVPPTFGQGTKVEIKGGGGSGGGGSGGGGSQVQLVQSGAEVKKPGASVKVSCKASGYTFTDYEMHWVRQAPGQGLEWMGALDPKTGDTAYSQKFKGRVTLTADTSTSTVYMELSSLRSEDTAVYYCTRFYSYTYWGQGTLVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 55 - protein, 494 aa, synthetic construct
MALPVTALLLPLALLLHAARPDYKDDDDKDIVMTQTPLSLSVTPGQPASISCRSSQSLVHSNGNTYLHWYLQKPGQSPQLLIYKVSNRFSGVPDRFSGSGSGTDFTLKISRVEAEDVGVYYCSQNTHVPPTFGQGTKVEIKGGGGSGGGGSGGGGSQVQLVQSGAEVKKPGASVKVSCKASGYTFTSYEMHWVRQAPGQGLEWMGALDPKTGDTAYSQKFKGRVTLTADTSTSTVYMELSSLRSEDTAVYYCTRFYSYTYWGQGTLVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 56 - protein, 494 aa, synthetic construct
MALPVTALLLPLALLLHAARPDYKDDDDKDIVMTQTPLSLSVTPGQPASISCRSSQSLVHSNGNTYLHWYLQKPGQSPQLLIYKVSNRFSGVPDRFSGSGSGTDFTLKISRVEAEDVGVYYCSQNTHVPPTFGQGTKVEIKGGGGSGGGGSGGGGSQVQLVQSGAEVKKPGASVKVSCKASGYTFTDYEMHWVRQAPGQGLEWMGALDPKTGDTAYSQKFQGRVTLTADTSTSTVYMELSSLRSEDTAVYYCTRFYSYTYWGQGTLVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 57 - protein, 494 aa, synthetic construct [sequence omitted]

SEQ ID NO: 58 - protein, 494 aa, synthetic construct
MALPVTALLLPLALLLHAARPDYKDDDDKDIVMTQTPLSLSVTPGQPASISCRSSQSLVHSNGNTYLHWYLQKPGQSPQLLIYKVSSRFSGVPDRFSGSGSGTDFTLKISRVEAEDVGVYYCSQNTHVPPTFGQGTKVEIKGGGGSGGGGSGGGGSQVQLVQSGAEVKKPGASVKVSCKASGYTFTDYEMHWVRQAPGQGLEWMGALDPKTGDTAYSQKFKGRVTLTADTSTSTVYMELSSLRSEDTAVYYCTRFYSYTYWGQGTLVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 59 - protein, 494 aa, synthetic construct
MALPVTALLLPLALLLHAARPDYKDDDDKDIVMTQTPLSLSVTPGQPASISCRSSQSLVHSNGNTYLHWYLQKPGQSPQLLIYKVSSRFSGVPDRFSGSGSGTDFTLKISRVEAEDVGVYYCSQNTHVPPTFGQGTKVEIKGGGGSGGGGSGGGGSQVQLVQSGAEVKKPGASVKVSCKASGYTFTSYEMHWVRQAPGQGLEWMGALDPKTGDTAYSQKFKGRVTLTADTSTSTVYMELSSLRSEDTAVYYCTRFYSYTYWGQGTLVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 60 - protein, 494 aa, synthetic construct
MALPVTALLLPLALLLHAARPDYKDDDDKDIVMTQTPLSLSVTPGQPASISCRSSQSLVHSNGNTYLHWYLQKPGQSPQLLIYKVSSRFSGVPDRFSGSGSGTDFTLKISRVEAEDVGVYYCSQNTHVPPTFGQGTKVEIKGGGGSGGGGSGGGGSQVQLVQSGAEVKKPGASVKVSCKASGYTFTDYEMHWVRQAPGQGLEWMGALDPKTGDTAYSQKFQGRVTLTADTSTSTVYMELSSLRSEDTAVYYCTRFYSYTYWGQGTLVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 61 - protein, 494 aa, synthetic construct
MALPVTALLLPLALLLHAARPDYKDDDDKDIVMTQTPLSLSVTPGQPASISCRSSQSLVHSNGNTYLHWYLQKPGQSPQLLIYKVSSRFSGVPDRFSGSGSGTDFTLKISRVEAEDVGVYYCSQNTHVPPTFGQGTKVEIKGGGGSGGGGSGGGGSQVQLVQSGAEVKKPGASVKVSCKASGYTFTSYEMHWVRQAPGQGLEWMGALDPKTGDTAYSQKFQGRVTLTADTSTSTVYMELSSLRSEDTAVYYCTRFYSYTYWGQGTLVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 62 - protein, 242 aa, synthetic construct
DIVMTQTPLSLSVTPGQPASISCRSSQSLVHSNGNTYLHWYLQKPGQSPQLLIYKVSNRFSGVPDRFSGSGSGTDFTLKISRVEAEDVGVYYCSQNTHVPPTFGQGTKVEIKGGGGSGGGGSGGGGSQVQLVQSGAEVKKPGASVKVSCKASGYTFTSYEMHWVRQAPGQGLEWMGALDPSTGDTAYSQKFQGRVTLTADTSTSTVYMELSSLRSEDTAVYYCTRFYSYTYWGQGTLVTVSS

SEQ ID NO: 63 - protein, 242 aa, synthetic construct
DIVMTQTPLSLSVTPGQPASISCRSSQSLVHSNGNTYLHWYLQKPGQSPQLLIYKVSNRFSGVPDRFSGSGSGTDFTLKISRVEAEDVGVYYCSQNTHVPPTFGQGTKVEIKGGGGSGGGGSGGGGSQVQLVQSGAEVKKPGASVKVSCKASGYTFTSYEMHWVRQAPGQGLEWMGALDPKGGDTAYSQKFQGRVTLTADTSTSTVYMELSSLRSEDTAVYYCTRFYSYTYWGQGTLVTVSS

SEQ ID NO: 64 - protein, 242 aa, synthetic construct
DIVMTQTPLSLSVTPGQPASISCRSSQSLVHSNGNTYLHWYLQKPGQSPQLLIYKVSNRFSGVPDRFSGSGSGTDFTLKISRVEAEDVGVYYCSQNTHVPPTFGQGTKVEIKGGGGSGGGGSGGGGSQVQLVQSGAEVKKPGASVKVSCKASGYTFTSYEMHWVRQAPGQGLEWMGALDPSGGDTAYSQKFQGRVTLTADTSTSTVYMELSSLRSEDTAVYYCTRFYSYTYWGQGTLVTVSS

SEQ ID NO: 65 - protein, 242 aa, synthetic construct
DIVMTQTPLSLSVTPGQPASISCRSSQSLVHSNGNTYLHWYLQKPGQSPQLLIYKVSNRFSGVPDRFSGSGSGTDFTLKISRVEAEDVGVYYCSQNTHVPPTFGQGTKVEIKGGGGSGGGGSGGGGSQVQLVQSGAEVKKPGASVKVSCKASGYTFTSYEMHWVRQAPGQGLEWMGALDPKTGSTAYSQKFQGRVTLTADTSTSTVYMELSSLRSEDTAVYYCTRFYSYTYWGQGTLVTVSS

SEQ ID NO: 66 - protein, 242 aa, synthetic construct
DIVMTQTPLSLSVTPGQPASISCRSSQSLVHSNGNTYLHWYLQKPGQSPQLLIYKVSNRFSGVPDRFSGSGSGTDFTLKISRVEAEDVGVYYCSQNTHVPPTFGQGTKVEIKGGGGSGGGGSGGGGSQVQLVQSGAEVKKPGASVKVSCKASGYTFTSYEMHWVRQAPGQGLEWMGALDPSGGSTAYSQKFQGRVTLTADTSTSTVYMELSSLRSEDTAVYYCTRFYSYTYWGQGTLVTVSS

SEQ ID NO: 67 - protein, 242 aa, synthetic construct
DIVMTQTPLSLSVTPGQPASISCRSSQSLVHSNGKTYLHWYLQKPGQSPQLLIYKVSSRFSGVPDRFSGSGSGTDFTLKISRVEAEDVGVYYCSQNTHVPPTFGQGTKVEIKGGGGSGGGGSGGGGSQVQLVQSGAEVKKPGASVKVSCKASGYTFTDYEMHWVRQAPGQGLEWMGALDPKTGDTAYSQKFKGRVTLTADTSTSTVYMELSSLRSEDTAVYYCTRFYSYTYWGQGTLVTVSS

SEQ ID NO: 68 - protein, 242 aa, synthetic construct
DIVMTQTPLSLSVTPGQPASISCRSSQSLVHSNGKTYLHWYLQKPGQSPQLLIYKVSSRFSGVPDRFSGSGSGTDFTLKISRVEAEDVGVYYCSQNTHVPPTFGQGTKVEIKGGGGSGGGGSGGGGSQVQLVQSGAEVKKPGASVKVSCKASGYTFTSYEMHWVRQAPGQGLEWMGALDPSTGDTAYSQKFQGRVTLTADTSTSTVYMELSSLRSEDTAVYYCTRFYSYTYWGQGTLVTVSS

SEQ ID NO: 69 - protein, 242 aa, synthetic construct
DIVMTQTPLSLSVTPGQPASISCRSSQSLVHSNGKTYLHWYLQKPGQSPQLLIYKVSSRFSGVPDRFSGSGSGTDFTLKISRVEAEDVGVYYCSQNTHVPPTFGQGTKVEIKGGGGSGGGGSGGGGSQVQLVQSGAEVKKPGASVKVSCKASGYTFTSYEMHWVRQAPGQGLEWMGALDPKGGDTAYSQKFQGRVTLTADTSTSTVYMELSSLRSEDTAVYYCTRFYSYTYWGQGTLVTVSS

SEQ ID NO: 70 - protein, 242 aa, synthetic construct
DIVMTQTPLSLSVTPGQPASISCRSSQSLVHSNGKTYLHWYLQKPGQSPQLLIYKVSSRFSGVPDRFSGSGSGTDFTLKISRVEAEDVGVYYCSQNTHVPPTFGQGTKVEIKGGGGSGGGGSGGGGSQVQLVQSGAEVKKPGASVKVSCKASGYTFTSYEMHWVRQAPGQGLEWMGALDPSGGDTAYSQKFQGRVTLTADTSTSTVYMELSSLRSEDTAVYYCTRFYSYTYWGQGTLVTVSS

SEQ ID NO: 71 - protein, 242 aa, synthetic construct
DIVMTQTPLSLSVTPGQPASISCRSSQSLVHSNGKTYLHWYLQKPGQSPQLLIYKVSSRFSGVPDRFSGSGSGTDFTLKISRVEAEDVGVYYCSQNTHVPPTFGQGTKVEIKGGGGSGGGGSGGGGSQVQLVQSGAEVKKPGASVKVSCKASGYTFTSYEMHWVRQAPGQGLEWMGALDPKTGSTAYSQKFQGRVTLTADTSTSTVYMELSSLRSEDTAVYYCTRFYSYTYWGQGTLVTVSS

SEQ ID NO: 72 - protein, 242 aa, synthetic construct
DIVMTQTPLSLSVTPGQPASISCRSSQSLVHSNGKTYLHWYLQKPGQSPQLLIYKVSSRFSGVPDRFSGSGSGTDFTLKISRVEAEDVGVYYCSQNTHVPPTFGQGTKVEIKGGGGSGGGGSGGGGSQVQLVQSGAEVKKPGASVKVSCKASGYTFTSYEMHWVRQAPGQGLEWMGALDPSGGSTAYSQKFQGRVTLTADTSTSTVYMELSSLRSEDTAVYYCTRFYSYTYWGQGTLVTVSS

SEQ ID NO: 73 - protein, 242 aa, synthetic construct
DIVMTQTPLSLSVTPGQPASISCRSSQSLVHSNGNTYLHWYLQKPGQSPQLLIYKVSSRFSGVPDRFSGSGSGTDFTLKISRVEAEDVGVYYCMQNTHVPPTFGQGTKVEIKGGGGSGGGGSGGGGSQVQLVQSGAEVKKPGASVKVSCKASGYTFTDYEMHWVRQAPGQGLEWMGALDPKTGDTAYSQKFKGRVTLTADTSTSTVYMELSSLRSEDTAVYYCTRFYSYTYWGQGTLVTVSS

SEQ ID NO: 74 - protein, 242 aa, synthetic construct
DIVMTQTPLSLSVTPGQPASISCRSSQSLVHSNGNTYLHWYLQKPGQSPQLLIYKVSSRFSGVPDRFSGSGSGTDFTLKISRVEAEDVGVYYCMQNTHVPPTFGQGTKVEIKGGGGSGGGGSGGGGSQVQLVQSGAEVKKPGASVKVSCKASGYTFTSYEMHWVRQAPGQGLEWMGALDPSTGDTAYSQKFQGRVTLTADTSTSTVYMELSSLRSEDTAVYYCTRFYSYTYWGQGTLVTVSS

SEQ ID NO: 75 - protein, 242 aa, synthetic construct
DIVMTQTPLSLSVTPGQPASISCRSSQSLVHSNGNTYLHWYLQKPGQSPQLLIYKVSSRFSGVPDRFSGSGSGTDFTLKISRVEAEDVGVYYCMQNTHVPPTFGQGTKVEIKGGGGSGGGGSGGGGSQVQLVQSGAEVKKPGASVKVSCKASGYTFTSYEMHWVRQAPGQGLEWMGALDPKGGDTAYSQKFQGRVTLTADTSTSTVYMELSSLRSEDTAVYYCTRFYSYTYWGQGTLVTVSS

SEQ ID NO: 76 - protein, 242 aa, synthetic construct
DIVMTQTPLSLSVTPGQPASISCRSSQSLVHSNGNTYLHWYLQKPGQSPQLLIYKVSSRFSGVPDRFSGSGSGTDFTLKISRVEAEDVGVYYCMQNTHVPPTFGQGTKVEIKGGGGSGGGGSGGGGSQVQLVQSGAEVKKPGASVKVSCKASGYTFTSYEMHWVRQAPGQGLEWMGALDPSGGDTAYSQKFQGRVTLTADTSTSTVYMELSSLRSEDTAVYYCTRFYSYTYWGQGTLVTVSS

SEQ ID NO: 77 - protein, 242 aa, synthetic construct
DIVMTQTPLSLSVTPGQPASISCRSSQSLVHSNGNTYLHWYLQKPGQSPQLLIYKVSSRFSGVPDRFSGSGSGTDFTLKISRVEAEDVGVYYCMQNTHVPPTFGQGTKVEIKGGGGSGGGGSGGGGSQVQLVQSGAEVKKPGASVKVSCKASGYTFTSYEMHWVRQAPGQGLEWMGALDPKTGSTAYSQKFQGRVTLTADTSTSTVYMELSSLRSEDTAVYYCTRFYSYTYWGQGTLVTVSS

SEQ ID NO: 78 - protein, 242 aa, synthetic construct
DIVMTQTPLSLSVTPGQPASISCRSSQSLVHSNGNTYLHWYLQKPGQSPQLLIYKVSSRFSGVPDRFSGSGSGTDFTLKISRVEAEDVGVYYCMQNTHVPPTFGQGTKVEIKGGGGSGGGGSGGGGSQVQLVQSGAEVKKPGASVKVSCKASGYTFTSYEMHWVRQAPGQGLEWMGALDPSGGSTAYSQKFQGRVTLTADTSTSTVYMELSSLRSEDTAVYYCTRFYSYTYWGQGTLVTVSS

SEQ ID NO: 79 - protein, 242 aa, synthetic construct
DIVMTQTPLSLSVTPGQPASISCRSSQSLVHSNGNTYLHWYLQKPGQSPQLLIYKVSSRFSGVPDRFSGSGSGTDFTLKISRVEAEDVGVYYCSQGTHVPPTFGQGTKVEIKGGGGSGGGGSGGGGSQVQLVQSGAEVKKPGASVKVSCKASGYTFTDYEMHWVRQAPGQGLEWMGALDPKTGDTAYSQKFKGRVTLTADTSTSTVYMELSSLRSEDTAVYYCTRFYSYTYWGQGTLVTVSS

SEQ ID NO: 80 - protein, 242 aa, synthetic construct
DIVMTQTPLSLSVTPGQPASISCRSSQSLVHSNGNTYLHWYLQKPGQSPQLLIYKVSSRFSGVPDRFSGSGSGTDFTLKISRVEAEDVGVYYCSQGTHVPPTFGQGTKVEIKGGGGSGGGGSGGGGSQVQLVQSGAEVKKPGASVKVSCKASGYTFTSYEMHWVRQAPGQGLEWMGALDPSTGDTAYSQKFQGRVTLTADTSTSTVYMELSSLRSEDTAVYYCTRFYSYTYWGQGTLVTVSS

SEQ ID NO: 81 - protein, 242 aa, synthetic construct
DIVMTQTPLSLSVTPGQPASISCRSSQSLVHSNGNTYLHWYLQKPGQSPQLLIYKVSSRFSGVPDRFSGSGSGTDFTLKISRVEAEDVGVYYCSQGTHVPPTFGQGTKVEIKGGGGSGGGGSGGGGSQVQLVQSGAEVKKPGASVKVSCKASGYTFTSYEMHWVRQAPGQGLEWMGALDPKGGDTAYSQKFQGRVTLTADTSTSTVYMELSSLRSEDTAVYYCTRFYSYTYWGQGTLVTVSS

SEQ ID NO: 82 - protein, 242 aa, synthetic construct
DIVMTQTPLSLSVTPGQPASISCRSSQSLVHSNGNTYLHWYLQKPGQSPQLLIYKVSSRFSGVPDRFSGSGSGTDFTLKISRVEAEDVGVYYCSQGTHVPPTFGQGTKVEIKGGGGSGGGGSGGGGSQVQLVQSGAEVKKPGASVKVSCKASGYTFTSYEMHWVRQAPGQGLEWMGALDPSGGDTAYSQKFQGRVTLTADTSTSTVYMELSSLRSEDTAVYYCTRFYSYTYWGQGTLVTVSS

SEQ ID NO: 83 - protein, 242 aa, synthetic construct
DIVMTQTPLSLSVTPGQPASISCRSSQSLVHSNGNTYLHWYLQKPGQSPQLLIYKVSSRFSGVPDRFSGSGSGTDFTLKISRVEAEDVGVYYCSQGTHVPPTFGQGTKVEIKGGGGSGGGGSGGGGSQVQLVQSGAEVKKPGASVKVSCKASGYTFTSYEMHWVRQAPGQGLEWMGALDPKTGSTAYSQKFQGRVTLTADTSTSTVYMELSSLRSEDTAVYYCTRFYSYTYWGQGTLVTVSS

SEQ ID NO: 84 - protein, 242 aa, synthetic construct
DIVMTQTPLSLSVTPGQPASISCRSSQSLVHSNGNTYLHWYLQKPGQSPQLLIYKVSSRFSGVPDRFSGSGSGTDFTLKISRVEAEDVGVYYCSQGTHVPPTFGQGTKVEIKGGGGSGGGGSGGGGSQVQLVQSGAEVKKPGASVKVSCKASGYTFTSYEMHWVRQAPGQGLEWMGALDPSGGSTAYSQKFQGRVTLTADTSTSTVYMELSSLRSEDTAVYYCTRFYSYTYWGQGTLVTVSS

SEQ ID NO: 85 - protein, 242 aa, synthetic construct
DIVMTQTPLSLSVTPGQPASISCRSSQSLVHSNGNTYLHWYLQKPGQSPQLLIYKVSSRFSGVPDRFSGSGSGTDFTLKISRVEAEDVGVYYCSQNTHWPPTFGQGTKVEIKGGGGSGGGGSGGGGSQVQLVQSGAEVKKPGASVKVSCKASGYTFTDYEMHWVRQAPGQGLEWMGALDPKTGDTAYSQKFKGRVTLTADTSTSTVYMELSSLRSEDTAVYYCTRFYSYTYWGQGTLVTVSS

SEQ ID NO: 86 - protein, 242 aa, synthetic construct
DIVMTQTPLSLSVTPGQPASISCRSSQSLVHSNGNTYLHWYLQKPGQSPQLLIYKVSSRFSGVPDRFSGSGSGTDFTLKISRVEAEDVGVYYCSQNTHWPPTFGQGTKVEIKGGGGSGGGGSGGGGSQVQLVQSGAEVKKPGASVKVSCKASGYTFTSYEMHWVRQAPGQGLEWMGALDPSTGDTAYSQKFQGRVTLTADTSTSTVYMELSSLRSEDTAVYYCTRFYSYTYWGQGTLVTVSS

SEQ ID NO: 87 - protein, 242 aa, synthetic construct
DIVMTQTPLSLSVTPGQPASISCRSSQSLVHSNGNTYLHWYLQKPGQSPQLLIYKVSSRFSGVPDRFSGSGSGTDFTLKISRVEAEDVGVYYCSQNTHWPPTFGQGTKVEIKGGGGSGGGGSGGGGSQVQLVQSGAEVKKPGASVKVSCKASGYTFTSYEMHWVRQAPGQGLEWMGALDPKGGDTAYSQKFQGRVTLTADTSTSTVYMELSSLRSEDTAVYYCTRFYSYTYWGQGTLVTVSS

SEQ ID NO: 88 - protein, 242 aa, synthetic construct
DIVMTQTPLSLSVTPGQPASISCRSSQSLVHSNGNTYLHWYLQKPGQSPQLLIYKVSSRFSGVPDRFSGSGSGTDFTLKISRVEAEDVGVYYCSQNTHWPPTFGQGTKVEIKGGGGSGGGGSGGGGSQVQLVQSGAEVKKPGASVKVSCKASGYTFTSYEMHWVRQAPGQGLEWMGALDPSGGDTAYSQKFQGRVTLTADTSTSTVYMELSSLRSEDTAVYYCTRFYSYTYWGQGTLVTVSS

SEQ ID NO: 89 - protein, 242 aa, synthetic construct
DIVMTQTPLSLSVTPGQPASISCRSSQSLVHSNGNTYLHWYLQKPGQSPQLLIYKVSSRFSGVPDRFSGSGSGTDFTLKISRVEAEDVGVYYCSQNTHWPPTFGQGTKVEIKGGGGSGGGGSGGGGSQVQLVQSGAEVKKPGASVKVSCKASGYTFTSYEMHWVRQAPGQGLEWMGALDPKTGSTAYSQKFQGRVTLTADTSTSTVYMELSSLRSEDTAVYYCTRFYSYTYWGQGTLVTVSS

SEQ ID NO: 90 - protein, 242 aa, synthetic construct
DIVMTQTPLSLSVTPGQPASISCRSSQSLVHSNGNTYLHWYLQKPGQSPQLLIYKVSSRFSGVPDRFSGSGSGTDFTLKISRVEAEDVGVYYCSQNTHWPPTFGQGTKVEIKGGGGSGGGGSGGGGSQVQLVQSGAEVKKPGASVKVSCKASGYTFTSYEMHWVRQAPGQGLEWMGALDPSGGSTAYSQKFQGRVTLTADTSTSTVYMELSSLRSEDTAVYYCTRFYSYTYWGQGTLVTVSS

SEQ ID NO: 91 - protein, 242 aa, synthetic construct
DIVMTQTPLSLSVTPGQPASISCRSSQSLVHSNGNTYLHWYLQKPGQSPQLLIYKVSNRFSGVPDRFSGSGSGTDFTLKISRVEAEDVGVYYCMQNTHVPPTFGQGTKVEIKGGGGSGGGGSGGGGSQVQLVQSGAEVKKPGASVKVSCKASGYTFTDYEMHWVRQAPGQGLEWMGALDPKTGDTAYSQKFKGRVTLTADTSTSTVYMELSSLRSEDTAVYYCTRFYSYTYWGQGTLVTVSS

SEQ ID NO: 92 - protein, 242 aa, synthetic construct
DIVMTQTPLSLSVTPGQPASISCRSSQSLVHSNGNTYLHWYLQKPGQSPQLLIYKVSNRFSGVPDRFSGSGSGTDFTLKISRVEAEDVGVYYCMQNTHVPPTFGQGTKVEIKGGGGSGGGGSGGGGSQVQLVQSGAEVKKPGASVKVSCKASGYTFTSYEMHWVRQAPGQGLEWMGALDPSTGDTAYSQKFQGRVTLTADTSTSTVYMELSSLRSEDTAVYYCTRFYSYTYWGQGTLVTVSS

SEQ ID NO: 93 - protein, 242 aa, synthetic construct
DIVMTQTPLSLSVTPGQPASISCRSSQSLVHSNGNTYLHWYLQKPGQSPQLLIYKVSNRFSGVPDRFSGSGSGTDFTLKISRVEAEDVGVYYCMQNTHVPPTFGQGTKVEIKGGGGSGGGGSGGGGSQVQLVQSGAEVKKPGASVKVSCKASGYTFTSYEMHWVRQAPGQGLEWMGALDPKGGDTAYSQKFQGRVTLTADTSTSTVYMELSSLRSEDTAVYYCTRFYSYTYWGQGTLVTVSS

SEQ ID NO: 94 - protein, 242 aa, synthetic construct
DIVMTQTPLSLSVTPGQPASISCRSSQSLVHSNGNTYLHWYLQKPGQSPQLLIYKVSNRFSGVPDRFSGSGSGTDFTLKISRVEAEDVGVYYCMQNTHVPPTFGQGTKVEIKGGGGSGGGGSGGGGSQVQLVQSGAEVKKPGASVKVSCKASGYTFTSYEMHWVRQAPGQGLEWMGALDPSGGDTAYSQKFQGRVTLTADTSTSTVYMELSSLRSEDTAVYYCTRFYSYTYWGQGTLVTVSS

SEQ ID NO: 95 - protein, 242 aa, synthetic construct
DIVMTQTPLSLSVTPGQPASISCRSSQSLVHSNGNTYLHWYLQKPGQSPQLLIYKVSNRFSGVPDRFSGSGSGTDFTLKISRVEAEDVGVYYCMQNTHVPPTFGQGTKVEIKGGGGSGGGGSGGGGSQVQLVQSGAEVKKPGASVKVSCKASGYTFTSYEMHWVRQAPGQGLEWMGALDPKTGSTAYSQKFQGRVTLTADTSTSTVYMELSSLRSEDTAVYYCTRFYSYTYWGQGTLVTVSS

SEQ ID NO: 96 - protein, 242 aa, synthetic construct
DIVMTQTPLSLSVTPGQPASISCRSSQSLVHSNGNTYLHWYLQKPGQSPQLLIYKVSNRFSGVPDRFSGSGSGTDFTLKISRVEAEDVGVYYCMQNTHVPPTFGQGTKVEIKGGGGSGGGGSGGGGSQVQLVQSGAEVKKPGASVKVSCKASGYTFTSYEMHWVRQAPGQGLEWMGALDPSGGSTAYSQKFQGRVTLTADTSTSTVYMELSSLRSEDTAVYYCTRFYSYTYWGQGTLVTVSS

SEQ ID NO: 97 - protein, 494 aa, synthetic construct
MALPVTALLLPLALLLHAARPDYKDDDDKDIVMTQTPLSLSVTPGQPASISCRSSQSLVHSNGNTYLHWYLQKPGQSPQLLIYKVSNRFSGVPDRFSGSGSGTDFTLKISRVEAEDVGVYYCSQNTHVPPTFGQGTKVEIKGGGGSGGGGSGGGGSQVQLVQSGAEVKKPGASVKVSCKASGYTFTSYEMHWVRQAPGQGLEWMGALDPSTGDTAYSQKFQGRVTLTADTSTSTVYMELSSLRSEDTAVYYCTRFYSYTYWGQGTLVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 98 - protein, 494 aa, synthetic construct
MALPVTALLLPLALLLHAARPDYKDDDDKDIVMTQTPLSLSVTPGQPASISCRSSQSLVHSNGNTYLHWYLQKPGQSPQLLIYKVSNRFSGVPDRFSGSGSGTDFTLKISRVEAEDVGVYYCSQNTHVPPTFGQGTKVEIKGGGGSGGGGSGGGGSQVQLVQSGAEVKKPGASVKVSCKASGYTFTSYEMHWVRQAPGQGLEWMGALDPKGGDTAYSQKFQGRVTLTADTSTSTVYMELSSLRSEDTAVYYCTRFYSYTYWGQGTLVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 99 - protein, 494 aa, synthetic construct
MALPVTALLLPLALLLHAARPDYKDDDDKDIVMTQTPLSLSVTPGQPASISCRSSQSLVHSNGNTYLHWYLQKPGQSPQLLIYKVSNRFSGVPDRFSGSGSGTDFTLKISRVEAEDVGVYYCSQNTHVPPTFGQGTKVEIKGGGGSGGGGSGGGGSQVQLVQSGAEVKKPGASVKVSCKASGYTFTSYEMHWVRQAPGQGLEWMGALDPSGGDTAYSQKFQGRVTLTADTSTSTVYMELSSLRSEDTAVYYCTRFYSYTYWGQGTLVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 100 - protein, 494 aa, synthetic construct
MALPVTALLLPLALLLHAARPDYKDDDDKDIVMTQTPLSLSVTPGQPASISCRSSQSLVHSNGNTYLHWYLQKPGQSPQLLIYKVSNRFSGVPDRFSGSGSGTDFTLKISRVEAEDVGVYYCSQNTHVPPTFGQGTKVEIKGGGGSGGGGSGGGGSQVQLVQSGAEVKKPGASVKVSCKASGYTFTSYEMHWVRQAPGQGLEWMGALDPKTGSTAYSQKFQGRVTLTADTSTSTVYMELSSLRSEDTAVYYCTRFYSYTYWGQGTLVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 101 - protein, 494 aa, synthetic construct
MALPVTALLLPLALLLHAARPDYKDDDDKDIVMTQTPLSLSVTPGQPASISCRSSQSLVHSNGNTYLHWYLQKPGQSPQLLIYKVSNRFSGVPDRFSGSGSGTDFTLKISRVEAEDVGVYYCSQNTHVPPTFGQGTKVEIKGGGGSGGGGSGGGGSQVQLVQSGAEVKKPGASVKVSCKASGYTFTSYEMHWVRQAPGQGLEWMGALDPSGGSTAYSQKFQGRVTLTADTSTSTVYMELSSLRSEDTAVYYCTRFYSYTYWGQGTLVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 102 - protein, 494 aa, synthetic construct
MALPVTALLLPLALLLHAARPDYKDDDDKDIVMTQTPLSLSVTPGQPASISCRSSQSLVHSNGKTYLHWYLQKPGQSPQLLIYKVSSRFSGVPDRFSGSGSGTDFTLKISRVEAEDVGVYYCSQNTHVPPTFGQGTKVEIKGGGGSGGGGSGGGGSQVQLVQSGAEVKKPGASVKVSCKASGYTFTDYEMHWVRQAPGQGLEWMGALDPKTGDTAYSQKFKGRVTLTADTSTSTVYMELSSLRSEDTAVYYCTRFYSYTYWGQGTLVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 103 - protein, 494 aa, synthetic construct
MALPVTALLLPLALLLHAARPDYKDDDDKDIVMTQTPLSLSVTPGQPASISCRSSQSLVHSNGKTYLHWYLQKPGQSPQLLIYKVSSRFSGVPDRFSGSGSGTDFTLKISRVEAEDVGVYYCSQNTHVPPTFGQGTKVEIKGGGGSGGGGSGGGGSQVQLVQSGAEVKKPGASVKVSCKASGYTFTSYEMHWVRQAPGQGLEWMGALDPSTGDTAYSQKFQGRVTLTADTSTSTVYMELSSLRSEDTAVYYCTRFYSYTYWGQGTLVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 104 - protein, 494 aa, synthetic construct
MALPVTALLLPLALLLHAARPDYKDDDDKDIVMTQTPLSLSVTPGQPASISCRSSQSLVHSNGKTYLHWYLQKPGQSPQLLIYKVSSRFSGVPDRFSGSGSGTDFTLKISRVEAEDVGVYYCSQNTHVPPTFGQGTKVEIKGGGGSGGGGSGGGGSQVQLVQSGAEVKKPGASVKVSCKASGYTFTSYEMHWVRQAPGQGLEWMGALDPKGGDTAYSQKFQGRVTLTADTSTSTVYMELSSLRSEDTAVYYCTRFYSYTYWGQGTLVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 105 - protein, 494 aa, synthetic construct
MALPVTALLLPLALLLHAARPDYKDDDDKDIVMTQTPLSLSVTPGQPASISCRSSQSLVHSNGKTYLHWYLQKPGQSPQLLIYKVSSRFSGVPDRFSGSGSGTDFTLKISRVEAEDVGVYYCSQNTHVPPTFGQGTKVEIKGGGGSGGGGSGGGGSQVQLVQSGAEVKKPGASVKVSCKASGYTFTSYEMHWVRQAPGQGLEWMGALDPSGGDTAYSQKFQGRVTLTADTSTSTVYMELSSLRSEDTAVYYCTRFYSYTYWGQGTLVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 106 - protein, 494 aa, synthetic construct
MALPVTALLLPLALLLHAARPDYKDDDDKDIVMTQTPLSLSVTPGQPASISCRSSQSLVHSNGKTYLHWYLQKPGQSPQLLIYKVSSRFSGVPDRFSGSGSGTDFTLKISRVEAEDVGVYYCSQNTHVPPTFGQGTKVEIKGGGGSGGGGSGGGGSQVQLVQSGAEVKKPGASVKVSCKASGYTFTSYEMHWVRQAPGQGLEWMGALDPKTGSTAYSQKFQGRVTLTADTSTSTVYMELSSLRSEDTAVYYCTRFYSYTYWGQGTLVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 107 - protein, 494 aa, synthetic construct
MALPVTALLLPLALLLHAARPDYKDDDDKDIVMTQTPLSLSVTPGQPASISCRSSQSLVHSNGKTYLHWYLQKPGQSPQLLIYKVSSRFSGVPDRFSGSGSGTDFTLKISRVEAEDVGVYYCSQNTHVPPTFGQGTKVEIKGGGGSGGGGSGGGGSQVQLVQSGAEVKKPGASVKVSCKASGYTFTSYEMHWVRQAPGQGLEWMGALDPSGGSTAYSQKFQGRVTLTADTSTSTVYMELSSLRSEDTAVYYCTRFYSYTYWGQGTLVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 108 - protein, 494 aa, synthetic construct
MALPVTALLLPLALLLHAARPDYKDDDDKDIVMTQTPLSLSVTPGQPASISCRSSQSLVHSNGNTYLHWYLQKPGQSPQLLIYKVSSRFSGVPDRFSGSGSGTDFTLKISRVEAEDVGVYYCMQNTHVPPTFGQGTKVEIKGGGGSGGGGSGGGGSQVQLVQSGAEVKKPGASVKVSCKASGYTFTDYEMHWVRQAPGQGLEWMGALDPKTGDTAYSQKFKGRVTLTADTSTSTVYMELSSLRSEDTAVYYCTRFYSYTYWGQGTLVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 109 - protein, 494 aa, synthetic construct
MALPVTALLLPLALLLHAARPDYKDDDDKDIVMTQTPLSLSVTPGQPASISCRSSQSLVHSNGNTYLHWYLQKPGQSPQLLIYKVSSRFSGVPDRFSGSGSGTDFTLKISRVEAEDVGVYYCMQNTHVPPTFGQGTKVEIKGGGGSGGGGSGGGGSQVQLVQSGAEVKKPGASVKVSCKASGYTFTSYEMHWVRQAPGQGLEWMGALDPSTGDTAYSQKFQGRVTLTADTSTSTVYMELSSLRSEDTAVYYCTRFYSYTYWGQGTLVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 110 - protein, 494 aa, synthetic construct
MALPVTALLLPLALLLHAARPDYKDDDDKDIVMTQTPLSLSVTPGQPASISCRSSQSLVHSNGNTYLHWYLQKPGQSPQLLIYKVSSRFSGVPDRFSGSGSGTDFTLKISRVEAEDVGVYYCMQNTHVPPTFGQGTKVEIKGGGGSGGGGSGGGGSQVQLVQSGAEVKKPGASVKVSCKASGYTFTSYEMHWVRQAPGQGLEWMGALDPKGGDTAYSQKFQGRVTLTADTSTSTVYMELSSLRSEDTAVYYCTRFYSYTYWGQGTLVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 111 - protein, 494 aa, synthetic construct
MALPVTALLLPLALLLHAARPDYKDDDDKDIVMTQTPLSLSVTPGQPASISCRSSQSLVHSNGNTYLHWYLQKPGQSPQLLIYKVSSRFSGVPDRFSGSGSGTDFTLKISRVEAEDVGVYYCMQNTHVPPTFGQGTKVEIKGGGGSGGGGSGGGGSQVQLVQSGAEVKKPGASVKVSCKASGYTFTSYEMHWVRQAPGQGLEWMGALDPSGGDTAYSQKFQGRVTLTADTSTSTVYMELSSLRSEDTAVYYCTRFYSYTYWGQGTLVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 112 - protein, 494 aa, synthetic construct
MALPVTALLLPLALLLHAARPDYKDDDDKDIVMTQTPLSLSVTPGQPASISCRSSQSLVHSNGNTYLHWYLQKPGQSPQLLIYKVSSRFSGVPDRFSGSGSGTDFTLKISRVEAEDVGVYYCMQNTHVPPTFGQGTKVEIKGGGGSGGGGSGGGGSQVQLVQSGAEVKKPGASVKVSCKASGYTFTSYEMHWVRQAPGQGLEWMGALDPKTGSTAYSQKFQGRVTLTADTSTSTVYMELSSLRSEDTAVYYCTRFYSYTYWGQGTLVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 113 - protein, 494 aa, synthetic construct
MALPVTALLLPLALLLHAARPDYKDDDDKDIVMTQTPLSLSVTPGQPASISCRSSQSLVHSNGNTYLHWYLQKPGQSPQLLIYKVSSRFSGVPDRFSGSGSGTDFTLKISRVEAEDVGVYYCMQNTHVPPTFGQGTKVEIKGGGGSGGGGSGGGGSQVQLVQSGAEVKKPGASVKVSCKASGYTFTSYEMHWVRQAPGQGLEWMGALDPSGGSTAYSQKFQGRVTLTADTSTSTVYMELSSLRSEDTAVYYCTRFYSYTYWGQGTLVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 114 - protein, 494 aa, synthetic construct
MALPVTALLLPLALLLHAARPDYKDDDDKDIVMTQTPLSLSVTPGQPASISCRSSQSLVHSNGNTYLHWYLQKPGQSPQLLIYKVSSRFSGVPDRFSGSGSGTDFTLKISRVEAEDVGVYYCSQGTHVPPTFGQGTKVEIKGGGGSGGGGSGGGGSQVQLVQSGAEVKKPGASVKVSCKASGYTFTDYEMHWVRQAPGQGLEWMGALDPKTGDTAYSQKFKGRVTLTADTSTSTVYMELSSLRSEDTAVYYCTRFYSYTYWGQGTLVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 115 - protein, 494 aa, synthetic construct
MALPVTALLLPLALLLHAARPDYKDDDDKDIVMTQTPLSLSVTPGQPASISCRSSQSLVHSNGNTYLHWYLQKPGQSPQLLIYKVSSRFSGVPDRFSGSGSGTDFTLKISRVEAEDVGVYYCSQGTHVPPTFGQGTKVEIKGGGGSGGGGSGGGGSQVQLVQSGAEVKKPGASVKVSCKASGYTFTSYEMHWVRQAPGQGLEWMGALDPSTGDTAYSQKFQGRVTLTADTSTSTVYMELSSLRSEDTAVYYCTRFYSYTYWGQGTLVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 116 - protein, 494 aa, synthetic construct
MALPVTALLLPLALLLHAARPDYKDDDDKDIVMTQTPLSLSVTPGQPASISCRSSQSLVHSNGNTYLHWYLQKPGQSPQLLIYKVSSRFSGVPDRFSGSGSGTDFTLKISRVEAEDVGVYYCSQGTHVPPTFGQGTKVEIKGGGGSGGGGSGGGGSQVQLVQSGAEVKKPGASVKVSCKASGYTFTSYEMHWVRQAPGQGLEWMGALDPKGGDTAYSQKFQGRVTLTADTSTSTVYMELSSLRSEDTAVYYCTRFYSYTYWGQGTLVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 117 - protein, 494 aa, synthetic construct
MALPVTALLLPLALLLHAARPDYKDDDDKDIVMTQTPLSLSVTPGQPASISCRSSQSLVHSNGNTYLHWYLQKPGQSPQLLIYKVSSRFSGVPDRFSGSGSGTDFTLKISRVEAEDVGVYYCSQGTHVPPTFGQGTKVEIKGGGGSGGGGSGGGGSQVQLVQSGAEVKKPGASVKVSCKASGYTFTSYEMHWVRQAPGQGLEWMGALDPSGGDTAYSQKFQGRVTLTADTSTSTVYMELSSLRSEDTAVYYCTRFYSYTYWGQGTLVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 118 - protein, 494 aa, synthetic construct
MALPVTALLLPLALLLHAARPDYKDDDDKDIVMTQTPLSLSVTPGQPASISCRSSQSLVHSNGNTYLHWYLQKPGQSPQLLIYKVSSRFSGVPDRFSGSGSGTDFTLKISRVEAEDVGVYYCSQGTHVPPTFGQGTKVEIKGGGGSGGGGSGGGGSQVQLVQSGAEVKKPGASVKVSCKASGYTFTSYEMHWVRQAPGQGLEWMGALDPKTGSTAYSQKFQGRVTLTADTSTSTVYMELSSLRSEDTAVYYCTRFYSYTYWGQGTLVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 119 - protein, 494 aa, synthetic construct
MALPVTALLLPLALLLHAARPDYKDDDDKDIVMTQTPLSLSVTPGQPASISCRSSQSLVHSNGNTYLHWYLQKPGQSPQLLIYKVSSRFSGVPDRFSGSGSGTDFTLKISRVEAEDVGVYYCSQGTHVPPTFGQGTKVEIKGGGGSGGGGSGGGGSQVQLVQSGAEVKKPGASVKVSCKASGYTFTSYEMHWVRQAPGQGLEWMGALDPSGGSTAYSQKFQGRVTLTADTSTSTVYMELSSLRSEDTAVYYCTRFYSYTYWGQGTLVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 120 - protein, 494 aa, synthetic construct
MALPVTALLLPLALLLHAARPDYKDDDDKDIVMTQTPLSLSVTPGQPASISCRSSQSLVHSNGNTYLHWYLQKPGQSPQLLIYKVSSRFSGVPDRFSGSGSGTDFTLKISRVEAEDVGVYYCSQNTHWPPTFGQGTKVEIKGGGGSGGGGSGGGGSQVQLVQSGAEVKKPGASVKVSCKASGYTFTDYEMHWVRQAPGQGLEWMGALDPKTGDTAYSQKFKGRVTLTADTSTSTVYMELSSLRSEDTAVYYCTRFYSYTYWGQGTLVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 121 - protein, 494 aa, synthetic construct
MALPVTALLLPLALLLHAARPDYKDDDDKDIVMTQTPLSLSVTPGQPASISCRSSQSLVHSNGNTYLHWYLQKPGQSPQLLIYKVSSRFSGVPDRFSGSGSGTDFTLKISRVEAEDVGVYYCSQNTHWPPTFGQGTKVEIKGGGGSGGGGSGGGGSQVQLVQSGAEVKKPGASVKVSCKASGYTFTSYEMHWVRQAPGQGLEWMGALDPSTGDTAYSQKFQGRVTLTADTSTSTVYMELSSLRSEDTAVYYCTRFYSYTYWGQGTLVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 122 - protein, 494 aa, synthetic construct
MALPVTALLLPLALLLHAARPDYKDDDDKDIVMTQTPLSLSVTPGQPASISCRSSQSLVHSNGNTYLHWYLQKPGQSPQLLIYKVSSRFSGVPDRFSGSGSGTDFTLKISRVEAEDVGVYYCSQNTHWPPTFGQGTKVEIKGGGGSGGGGSGGGGSQVQLVQSGAEVKKPGASVKVSCKASGYTFTSYEMHWVRQAPGQGLEWMGALDPKGGDTAYSQKFQGRVTLTADTSTSTVYMELSSLRSEDTAVYYCTRFYSYTYWGQGTLVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 123 - protein, 494 aa, synthetic construct
MALPVTALLLPLALLLHAARPDYKDDDDKDIVMTQTPLSLSVTPGQPASISCRSSQSLVHSNGNTYLHWYLQKPGQSPQLLIYKVSSRFSGVPDRFSGSGSGTDFTLKISRVEAEDVGVYYCSQNTHWPPTFGQGTKVEIKGGGGSGGGGSGGGGSQVQLVQSGAEVKKPGASVKVSCKASGYTFTSYEMHWVRQAPGQGLEWMGALDPSGGDTAYSQKFQGRVTLTADTSTSTVYMELSSLRSEDTAVYYCTRFYSYTYWGQGTLVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 124 - protein, 494 aa, synthetic construct
MALPVTALLLPLALLLHAARPDYKDDDDKDIVMTQTPLSLSVTPGQPASISCRSSQSLVHSNGNTYLHWYLQKPGQSPQLLIYKVSSRFSGVPDRFSGSGSGTDFTLKISRVEAEDVGVYYCSQNTHWPPTFGQGTKVEIKGGGGSGGGGSGGGGSQVQLVQSGAEVKKPGASVKVSCKASGYTFTSYEMHWVRQAPGQGLEWMGALDPKTGSTAYSQKFQGRVTLTADTSTSTVYMELSSLRSEDTAVYYCTRFYSYTYWGQGTLVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 125 - protein, 494 aa, synthetic construct
MALPVTALLLPLALLLHAARPDYKDDDDKDIVMTQTPLSLSVTPGQPASISCRSSQSLVHSNGNTYLHWYLQKPGQSPQLLIYKVSSRFSGVPDRFSGSGSGTDFTLKISRVEAEDVGVYYCSQNTHWPPTFGQGTKVEIKGGGGSGGGGSGGGGSQVQLVQSGAEVKKPGASVKVSCKASGYTFTSYEMHWVRQAPGQGLEWMGALDPSGGSTAYSQKFQGRVTLTADTSTSTVYMELSSLRSEDTAVYYCTRFYSYTYWGQGTLVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 126 - protein, 494 aa, synthetic construct
MALPVTALLLPLALLLHAARPDYKDDDDKDIVMTQTPLSLSVTPGQPASISCRSSQSLVHSNGNTYLHWYLQKPGQSPQLLIYKVSNRFSGVPDRFSGSGSGTDFTLKISRVEAEDVGVYYCMQNTHVPPTFGQGTKVEIKGGGGSGGGGSGGGGSQVQLVQSGAEVKKPGASVKVSCKASGYTFTDYEMHWVRQAPGQGLEWMGALDPKTGDTAYSQKFKGRVTLTADTSTSTVYMELSSLRSEDTAVYYCTRFYSYTYWGQGTLVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 127 - protein, 494 aa, synthetic construct
MALPVTALLLPLALLLHAARPDYKDDDDKDIVMTQTPLSLSVTPGQPASISCRSSQSLVHSNGNTYLHWYLQKPGQSPQLLIYKVSNRFSGVPDRFSGSGSGTDFTLKISRVEAEDVGVYYCMQNTHVPPTFGQGTKVEIKGGGGSGGGGSGGGGSQVQLVQSGAEVKKPGASVKVSCKASGYTFTSYEMHWVRQAPGQGLEWMGALDPSTGDTAYSQKFQGRVTLTADTSTSTVYMELSSLRSEDTAVYYCTRFYSYTYWGQGTLVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 128 - protein, 494 aa, synthetic construct
MALPVTALLLPLALLLHAARPDYKDDDDKDIVMTQTPLSLSVTPGQPASISCRSSQSLVHSNGNTYLHWYLQKPGQSPQLLIYKVSNRFSGVPDRFSGSGSGTDFTLKISRVEAEDVGVYYCMQNTHVPPTFGQGTKVEIKGGGGSGGGGSGGGGSQVQLVQSGAEVKKPGASVKVSCKASGYTFTSYEMHWVRQAPGQGLEWMGALDPKGGDTAYSQKFQGRVTLTADTSTSTVYMELSSLRSEDTAVYYCTRFYSYTYWGQGTLVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 129 - protein, 494 aa, synthetic construct
MALPVTALLLPLALLLHAARPDYKDDDDKDIVMTQTPLSLSVTPGQPASISCRSSQSLVHSNGNTYLHWYLQKPGQSPQLLIYKVSNRFSGVPDRFSGSGSGTDFTLKISRVEAEDVGVYYCMQNTHVPPTFGQGTKVEIKGGGGSGGGGSGGGGSQVQLVQSGAEVKKPGASVKVSCKASGYTFTSYEMHWVRQAPGQGLEWMGALDPSGGDTAYSQKFQGRVTLTADTSTSTVYMELSSLRSEDTAVYYCTRFYSYTYWGQGTLVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 130 - protein, 494 aa, synthetic construct
MALPVTALLLPLALLLHAARPDYKDDDDKDIVMTQTPLSLSVTPGQPASISCRSSQSLVHSNGNTYLHWYLQKPGQSPQLLIYKVSNRFSGVPDRFSGSGSGTDFTLKISRVEAEDVGVYYCMQNTHVPPTFGQGTKVEIKGGGGSGGGGSGGGGSQVQLVQSGAEVKKPGASVKVSCKASGYTFTSYEMHWVRQAPGQGLEWMGALDPKTGSTAYSQKFQGRVTLTADTSTSTVYMELSSLRSEDTAVYYCTRFYSYTYWGQGTLVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 131 - protein, 494 aa, synthetic construct
MALPVTALLLPLALLLHAARPDYKDDDDKDIVMTQTPLSLSVTPGQPASISCRSSQSLVHSNGNTYLHWYLQKPGQSPQLLIYKVSNRFSGVPDRFSGSGSGTDFTLKISRVEAEDVGVYYCMQNTHVPPTFGQGTKVEIKGGGGSGGGGSGGGGSQVQLVQSGAEVKKPGASVKVSCKASGYTFTSYEMHWVRQAPGQGLEWMGALDPSGGSTAYSQKFQGRVTLTADTSTSTVYMELSSLRSEDTAVYYCTRFYSYTYWGQGTLVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 132 - protein, 10 aa, synthetic construct
  VARIANT 6: note = S or D
GYTFTXYEMH

SEQ ID NO: 133 - protein, 17 aa, synthetic construct
  VARIANT 5: note = S or K
  VARIANT 6: note = G or T
  VARIANT 8: note = S or D
  VARIANT 16: note = Q or K
ALDPXXGXTAYSQKFXG

SEQ ID NO: 134 - protein, 16 aa, synthetic construct
  VARIANT 12: note = N or K
RSSQSLVHSNGXTYLH

SEQ ID NO: 135 - protein, 7 aa, synthetic construct
  VARIANT 4: note = N or S
KVSXRFS

SEQ ID NO: 136 - protein, 9 aa, synthetic construct
  VARIANT 1: note = S or M
  VARIANT 3: note = N or G
  VARIANT 6: note = W or V
XQXTHXPPT

SEQ ID NO: 137 - protein, 112 aa, synthetic construct
DIVMTQTPLSLSVTPGQPASISCRSSQSLVHSNGNTYLHWYLQKPGQSPQLLIYKVSNRFSGVPDRFSGSGSGTDFTLKISRVEAEDVGVYYCMQNTHVPPTFGQGTKVEIK

SEQ ID NO: 138 - protein, 112 aa, synthetic construct
DVVMTQTPLSLPVSLGDQASISCRSSQSLVHSNGNTYLHWYLQKPGQSPKLLIYKVSNRFSGVPDRFSGSGSGTDFTLKISRVEAEDLGVYFCSQNTHVPPTFGSGTKLEIK

SEQ ID NO: 139 - protein, 115 aa, synthetic construct
QVQLQQSGAELVRPGASVKLSCKASGYTFTDYEMHWVKQTPVHGLKWIGALDPKTGDTAYSQKFKGKATLTADKSSSTAYMELRSLTSEDSAVYYCTRFYSYTYWGQGTLVTVSA

SEQ ID NO: 140 - protein, 5 aa, synthetic construct
GGGGS

(19) United States
(12) Patent Application Publication
(10) Pub. No.: US 2025/0297029 A1
(43) Pub. Date: Sep. 25, 2025

(54) ANTI-GPC3 CHIMERIC ANTIGEN RECEPTOR AND METHODS OF USE THEREOF

(71) Applicant: Nanjing Legend Biotech Co., Ltd., Nanjing (CN)

(72) Inventors: Qingling JIANG, Nanjing (CN); Shuai YANG, Nanjing (CN); Yafeng ZHANG, Nanjing (CN); Shu WU, Nanjing (CN); Xiaojie TU, Nanjing (CN)

(21) Appl. No.: 18/687,617

(22) PCT Filed: Sep. 6, 2022
(86) PCT No.: PCT/CN2022/117270

§ 371 Date: Feb. 28, 2024

(30) Foreign Application Priority Data
Sep. 6, 2021 (WO) PCT/CN2021/116702

(51) Int. Cl. C07K 16/30; A61K 40/11; A61K 40/31; A61K 40/42; A61P 35/00; C07K 14/705; C07K 14/725; C12N 5/0783

(52) U.S. Cl. CPC C07K 16/303; A61K 40/11; A61K 40/31; A61K 40/4261; A61P 35/00; C07K 14/7051; C07K 14/70578; C12N 5/0636; C07K 2317/24; C07K 2317/622; C07K 2317/92; C12N 2510/00

(57) ABSTRACT

Provided herein are antibodies and antigen-binding fragment thereof targeting GPC3, and chimeric antigen receptors having one or more anti-GPC3 antigen-binding fragments thereof. Further provided are engineered immune effector cells (e.g., T cells) expressing the chimeric antigen receptors and methods of use thereof.

### CROSS REFERENCE TO RELATED APPLICATION

This application is a 371 national stage application of PCT Application No. PCT/CN2022/117270, filed on Sep. 6, 2022, which claims priority to PCT Patent Application No. PCT/CN2021/116702, filed on Sep. 6, 2021. The disclosures of the aforementioned applications are hereby incorporated by reference in their entireties.

### SEQUENCE LISTING

This application contains a Sequence Listing that has been submitted electronically as an XML file named “51624-0008US1_SL_ST26.XML.” The XML file, created on Mar. 11, 2025, is 160,999 bytes in size. The material in the XML file is hereby incorporated by reference in its entirety.

### TECHNICAL FIELD

This disclosure relates to antibodies targeting GPC3, chimeric antigen receptors targeting GPC3, and methods of use thereof.

### BACKGROUND

Worldwide, liver cancers are the fourth most common cause of cancer-related death and rank sixth in terms of incident cases. On the basis of annual projections, the World Health Organization estimates that more than 1 million patients will die from liver cancer in 2030. Half of all liver cancer cases and deaths are estimated to occur in China (Chen W. Q. et al., CA Cancer J Clin. March-April 2016; 66(2):115-32). Hepatocellular carcinoma (HCC) accounts for the majority of primary liver cancers. Considerable challenges exist in the clinical management, most of HCC cases are diagnosed at advanced-stage that aren't suitable for curative treatment, such as surgical resection, liver transplantation, radiofrequency ablation or trans-arterial chemoembolization (Villanueva A. et al., N Engl J Med. 2019 Apr. 11; 380(15):1450-1462). Systemic therapies are recommended for these patients. However, the improvement in overall survival duration is limited and relapse is a frequent and expected event after systemic therapies (Tabrizian P. et al., Ann Surg. 2015 May; 261(5):947-55). Therefore, novel strategies for treatment of patients with advanced HCC are needed.

The field of cancer immunotherapy has been re-energized by the application of chimeric antigen receptor (CAR) T cell therapy in cancers. These CAR T cells are engineered to express synthetic receptors, which have a modular design with the following major components: an antigen-binding domain, a hinge, a transmembrane region and an intracellular signaling domain (Rafiq S. et al. Nat Rev Clin Oncol. 2020 March; 17(3):147-167). The extracellular antigen-binding domain may comprise a single chain variable fragment (scFv). Upon binding to the target tumor antigen, the CARs can activate the T cells to launch specific anti-tumor response in a major histocompatibility complexes (MHC)-independent manner. When designing CAR-modified immune effector cells, the targeted antigen is important.

Anti-GPC3 antibodies have been used for liver cancer detection. Although antibody-dependent cellular cytotoxicity (ADCC) and the complement-dependent cytotoxicity (CDC) research programs have been reported, no clinical use of anti-GPC3 antibodies have been approved. Only the GC33 antibody, codrituzumab (PCT Application No. PCT/JP2005/013103) entered clinical research. In Phase II clinical trial, codrituzumab was not found to be effective against liver cancer (Abou-Alfa G. K. et al., J Hepatol. 2016 August; 65(2):289-95). Thus, there is a need to further optimize and prepare new anti-GPC3 antibodies, which have good tumor killing activity and excellent clinical application prospects in the solid tumors.

### SUMMARY

The disclosure relates to antibodies and antigen-binding fragment thereof targeting GPC3, and chimeric antigen receptors (e.g., monovalent CAR, and multivalent CAR including bi-epitope CAR) having one or more anti-GPC3 antigen-binding fragments thereof Further provided are engineered immune effector cells (e.g., T cells) expressing the chimeric antigen receptors and methods of use thereof.

The disclosure also relates to anti-GPC3 CAR-T cell therapy for the treatment of cancer patients with GPC3-positive cancer, including e.g., liver cancer. Genetically engineered T cells can recognize and attack target cells. These T cells can be isolated from the host and genetically modified using e.g., suitable virus mediated or non-viral means of transfection. Thereafter, the modified T cells can be infused back into the patients as adoptive cell therapy.

The present disclosure further reduces immune response against these therapeutic agents in patients by further humanizing and optimizing antibody fragment sequences (e.g., scFv). These antibody fragments can be integrated into a CAR construct that will not elicit an immune response against these antibody fragments in patients, is safe for long term use, and maintains or has better clinical effectiveness as compared to known CAR-T therapy for treatment of hepatocellular carcinoma (HCC). The present disclosure further provides the use of T cells engineered to express a humanized antibody fragment that binds GPC3 integrated into a CAR to treat a solid cancer associated with expression of GPC3.

In one aspect, the disclosure relates to an antibody or antigen-binding fragment thereof that binds to GPC3, containing: a heavy chain variable region (VH) having VH complementarity determining regions (CDRs) 1, 2, and 3; and a light chain variable region (VL) having VL CDRs 1, 2, and 3, wherein: (a) the VH CDR1 has the amino acid sequence GYTFTX₁YEMH (SEQ ID NO: 132); (b) the VH CDR2 has the amino acid sequence ALDPX₂×₃GX₄TAYSQKFX₅G (SEQ ID NO: 133); (c) the VH CDR3 has the amino acid sequence FYSYTY(SEQ ID NO: 29); (d) the VL CDR1 has the amino acid sequence RSSQSLVHSNGX₆TYLH(SEQ ID NO: 134); (e) the VL CDR2 has the amino acid sequence KVSX₇RFS(SEQ ID NO: 135); and (f) the VL CDR3 has the amino acid sequence X₈QX₉THX₁₀PPT(SEQ ID NO: 136); and wherein: X₁ is S or D; X₂ is S or K; X₃ is G or T; X₄ is S or D; X₅ is Q or K; X₆ is N or K; X₇ is N or S; X₈ is S or M; X₉ is N or G; and X₁₀ is W or V. In some embodiments, the antibody or antigen-binding fragment is not an antibody or antigen-binding fragment containing VH CDRs 1, 2, 3 having amino acid sequences as set forth in SEQ ID NOs: 20, 22 and 29 respectively; and VL CDRs 1, 2, 3 having amino acid sequences as set forth in SEQ ID NOs: 30, 32 and 34 respectively. In some embodiments, VH CDR1 is not SEQ ID NO: 20. In some embodiments, VH CDR2 is not SEQ ID NO: 22. In some embodiments, VH CDR3 is not SEQ ID NO: 29. In some embodiments, VL CDR1 is not SEQ ID NO: 30. In some embodiments, VL CDR2 is not SEQ ID NO: 32. In some embodiments, VL CDR3 is not SEQ ID NO: 34.

In some embodiments, (a) the VH CDR1 amino acid sequence is selected from the group consisting of SEQ ID NOs: 20 and 21; (b) the VH CDR2 amino acid sequence is selected from the group consisting of SEQ ID NOs: 22-28; (c) the VH CDR3 amino acid sequence is SEQ ID NOs: 29; (d) the VL CDR1 amino acid sequence is selected from the group consisting of SEQ ID NOs: 30 and 31; (e) the VL CDR2 amino acid sequence is selected from the group consisting of SEQ ID NOs: 32 and 33; and (f) the VL CDR3 amino acid sequence is selected from the group consisting of SEQ ID NOs: 34-37.

In some embodiments, the VH CDRs 1, 2, 3 amino acid sequences are set forth in SEQ ID NOs: 20, 22 and 29 respectively; the VH CDRs 1, 2, 3 amino acid sequences are set forth in SEQ ID NOs: 21, 22 and 29 respectively; the VH CDRs 1, 2, 3 amino acid sequences are set forth in SEQ ID NOs: 20, 23 and 29 respectively; the VH CDRs 1, 2, 3 amino acid sequences are set forth in SEQ ID NOs: 21, 23 and 29 respectively; the VH CDRs 1, 2, 3 amino acid sequences are set forth in SEQ ID NOs: 21, 24 and 29 respectively; the VH CDRs 1, 2, 3 amino acid sequences are set forth in SEQ ID NOs: 21, 25 and 29 respectively; the VH CDRs 1, 2, 3 amino acid sequences are set forth in SEQ ID NOs: 21, 26 and 29 respectively; the VH CDRs 1, 2, 3 amino acid sequences are set forth in SEQ ID NOs: 21, 27 and 29 respectively; the VH CDRs 1, 2, 3 amino acid sequences are set forth in SEQ ID NOs: 21, 28 and 29 respectively. In some embodiments, the VL CDRs, 1, 2, and 3 amino acid sequences are one of the following: the VL CDRs 1, 2, 3 amino acid sequences are set forth in SEQ ID NOs: 30, 32 and 34 respectively; the VL CDRs 1, 2, 3 amino acid sequences are set forth in SEQ ID NOs: 30, 33 and 34 respectively; the VL CDRs 1, 2, 3 amino acid sequences are set forth in SEQ ID NOs: 31, 33 and 34 respectively; the VL CDRs 1, 2, 3 amino acid sequences are set forth in SEQ ID NOs: 30, 33 and 35 respectively; the VL CDRs 1, 2, 3 amino acid sequences are set forth in SEQ ID NOs: 30, 33 and 36 respectively; the VL CDRs 1, 2, 3 amino acid sequences are set forth in SEQ ID NOs: 30, 33 and 37 respectively; the VL CDRs 1, 2, 3 amino acid sequences are set forth in SEQ ID NOs: 30, 32 and 35 respectively.

In some embodiments, the VH CDRs 1, 2, and 3 amino acid sequences and the VL CDRs, 1, 2, and 3 amino acid sequences are one of the following: the VH CDRs 1, 2, 3 amino acid sequences are set forth in SEQ ID NOs: 20, 22, and 29 respectively, and the selected VL CDRs 1, 2, 3 amino acid sequences are set forth in SEQ ID NOs: 30, 32, and 34 respectively; the VH CDRs 1, 2, 3 amino acid sequences are set forth in SEQ ID NOs: 21, 22, and 29 respectively, and the selected VL CDRs 1, 2, 3 amino acid sequences are set forth in SEQ ID NOs: 30, 32 and 34 respectively; the VH CDRs 1, 2, 3 amino acid sequences are set forth in SEQ ID NOs: 20, 23 and 29 respectively, and the selected VL CDRs 1, 2, 3 amino acid sequences are set forth in SEQ ID NOs: 30, 32 and 34 respectively; the VH CDRs 1, 2, 3 amino acid sequences are set forth in SEQ ID NOs: 21, 23, and 29 respectively, and the selected VL CDRs 1, 2, 3 amino acid sequences are set forth in SEQ ID NOs: 30, 32 and 34 respectively; the VH CDRs 1, 2, 3 amino acid sequences are set forth in SEQ ID NOs: 20, 22 and 29 respectively, and the selected VL CDRs 1, 2, 3 amino acid sequences are set forth in SEQ ID NOs: 30, 33 and 34 respectively; the VH CDRs 1, 2, 3 amino acid sequences are set forth in SEQ ID NOs: 21, 22, and 29 respectively, and the selected VL CDRs 1, 2, 3 amino acid sequences are set forth in SEQ ID NOs: 30, 33 and 34 respectively; the VH CDRs 1, 2, 3 amino acid sequences are set forth in SEQ ID NOs: 20, 23, and 29 respectively, and the selected VL CDRs 1, 2, 3 amino acid sequences are set forth in SEQ ID NOs: 30, 33 and 34 respectively; the VH CDRs 1, 2, 3 amino acid sequences are set forth in SEQ ID NOs: 21, 23, and 29 respectively, and the selected VL CDRs 1, 2, 3 amino acid sequences are set forth in SEQ ID NOs: 30, 33 and 34 respectively; the VH CDRs 1, 2, 3 amino acid sequences are set forth in SEQ ID NOs: 21, 24, and 29 respectively, and the selected VL CDRs 1, 2, 3 amino acid sequences are set forth in SEQ ID NOs: 30, 33 and 34 respectively; the VH CDRs 1, 2, 3 amino acid sequences are set forth in SEQ ID NOs: 21, 25, and 29 respectively, and the selected VL CDRs 1, 2, 3 amino acid sequences are set forth in SEQ ID NOs: 30, 33 and 34 respectively; the VH CDRs 1, 2, 3 amino acid sequences are set forth in SEQ ID NOs: 21, 26, and 29 respectively, and the selected VL CDRs 1, 2, 3 amino acid sequences are set forth in SEQ ID NOs: 30, 33 and 34 respectively; the VH CDRs 1, 2, 3 amino acid sequences are set forth in SEQ ID NOs: 21, 27, and 29 respectively, and the selected VL CDRs 1, 2, 3 amino acid sequences are set forth in SEQ ID NOs: 30, 33 and 34 respectively; the VH CDRs 1, 2, 3 amino acid sequences are set forth in SEQ ID NOs: 21, 23 and 29 respectively, and the selected VL CDRs 1, 2, 3 amino acid sequences are set forth in SEQ ID NOs: 31, 33 and 34 respectively; the VH CDRs 1, 2, 3 amino acid sequences are set forth in SEQ ID NOs: 21, 23 and 29 respectively, and the selected VL CDRs 1, 2, 3 amino acid sequences are set forth in SEQ ID NOs: 30, 33 and 35 respectively; the VH CDRs 1, 2, 3 amino acid sequences are set forth in SEQ ID NOs: 21, 23 and 29 respectively, and the selected VL CDRs 1, 2, 3 amino acid sequences are set forth in SEQ ID NOs: 30, 33 and 36 respectively; the VH CDRs 1, 2, 3 amino acid sequences are set forth in SEQ ID NOs: 21, 23 and 29 respectively, and the selected VL CDRs 1, 2, 3 amino acid sequences are set forth in SEQ ID NOs: 30, 33 and 37 respectively.

In one aspect, the disclosure relates to an antibody or antigen-binding fragment thereof that binds to GPC3, containing a heavy chain variable region (VH) having an amino acid sequence that is at least 80%, 85%, 90%, 95% or 100% identical to a selected VH sequence, and a light chain variable region (VL) having an amino acid sequence that is at least 80%, 85%, 90%, 95% or 100% identical to a selected VL sequence. In some embodiments, the selected VH sequence is one of the following: the selected VH sequence is SEQ ID NO: 3; the selected VH sequence is SEQ ID NO: 5; the selected VH sequence is SEQ ID NO: 6; the selected VH sequence is SEQ ID NO: 7; the selected VH sequence is SEQ ID NO: 8; the selected VH sequence is SEQ ID NO: 11; the selected VH sequence is SEQ ID NO: 12; the selected VH sequence is SEQ ID NO: 13; the selected VH sequence is SEQ ID NO: 14; the selected VH sequence is SEQ ID NO: 15. In some embodiments, the selected VL sequence is one of the following: the selected VL sequence is SEQ ID NO: 4; the selected VL sequence is SEQ ID NO: 9; the selected VL sequence is SEQ ID NO: 16; the selected VL sequence is SEQ ID NO: 17; the selected VL sequence is SEQ ID NO: 18; the selected VL sequence is SEQ ID NO: 19; the selected VL sequence is SEQ ID NO: 137.

In some embodiments, the selected VH sequence is one of the following: the selected VH sequence is SEQ ID NO: 5, and the selected VL sequence is SEQ ID NO: 4; the selected VH sequence is SEQ ID NO: 6, and the selected VL sequence is SEQ ID NO: 4; the selected VH sequence is SEQ ID NO: 7, and the selected VL sequence is SEQ ID NO: 4; the selected VH sequence is SEQ ID NO: 8, and the selected VL sequence is SEQ ID NO: 4; the selected VH sequence is SEQ ID NO: 5, and the selected VL sequence is SEQ ID NO: 9; the selected VH sequence is SEQ ID NO: 6, and the selected VL sequence is SEQ ID NO: 9; the selected VH sequence is SEQ ID NO: 7, and the selected VL sequence is SEQ ID NO: 9; the selected VH sequence is SEQ ID NO: 8, and the selected VL sequence is SEQ ID NO: 9; the selected VH sequence is SEQ ID NO: 11, and the selected VL sequence is SEQ ID NO: 9; the selected VH sequence is SEQ ID NO: 12, and the selected VL sequence is SEQ ID NO: 9; the selected VH sequence is SEQ ID NO: 13, and the selected VL sequence is SEQ ID NO: 9; the selected VH sequence is SEQ ID NO: 14, and the selected VL sequence is SEQ ID NO: 9; the selected VH sequence is SEQ ID NO: 8, and the selected VL sequence is SEQ ID NO: 16; the selected VH sequence is SEQ ID NO: 8, and the selected VL sequence is SEQ ID NO: 17; the selected VH sequence is SEQ ID NO: 8, and the selected VL sequence is SEQ ID NO: 18; the selected VH sequence is SEQ ID NO: 8, and the selected VL sequence is SEQ ID NO: 19.

In one aspect, the disclosure relates to an antibody or antigen-binding fragment thereof that binds to GPC3, containing a heavy chain variable region (VH) having VH CDR1, VH CDR2, and VH CDR3 that are identical to VH CDR1, VH CDR2, and VH CDR3 of a selected VH sequence, and a light chain variable region (VL) having VL CDR1, VL CDR2, and VL CDR3 that are identical to VL CDR1, VL CDR2, and VL CDR3 of a selected VL sequence. In some embodiments, the selected VH sequence is one of the following: the selected VH sequence is SEQ ID NO: 3; the selected VH sequence is SEQ ID NO: 5; the selected VH sequence is SEQ ID NO: 6; the selected VH sequence is SEQ ID NO: 7; the selected VH sequence is SEQ ID NO: 8; the selected VH sequence is SEQ ID NO: 11; the selected VH sequence is SEQ ID NO: 12; the selected VH sequence is SEQ ID NO: 13; the selected VH sequence is SEQ ID NO: 14; the selected VH sequence is SEQ ID NO: 15. In some embodiments, the selected VL sequence is one of the following: the selected VL sequence is SEQ ID NO: 4; the selected VL sequence is SEQ ID NO: 9; the selected VL sequence is SEQ ID NO: 16; the selected VL sequence is SEQ ID NO: 17; the selected VL sequence is SEQ ID NO: 18; the selected VL sequence is SEQ ID NO: 19; the selected VL sequence is SEQ ID NO: 137.

In some embodiments, the antibody or antigen-binding fragment has a single-chain variable fragment (scFv). In some embodiments, the scFv has an amino acid sequence of SEQ ID NOs: 39-46 and 62-96.

In some embodiments, the antibody or antigen-binding fragment specifically binds to a human GPC3 peptide having a sequence that is at least 80%, 85%, 90%, 95%, or 100% identical to the amino acid sequence of SEQ ID NO: 1. In some embodiments, the antibody or antigen-binding fragment specifically binds to the extracellular domain (ECD) of human GPC3.

In some embodiments, the antibody or antigen-binding fragment is a humanized antibody or antigen-binding fragment thereof.

In some embodiments, the antibody or antigen-binding fragment is a chimeric antibody or antigen-binding fragment thereof or a human antibody or antigen-binding fragment thereof.

In one aspect, the disclosure relates to an antibody or antigen-binding fragment thereof that cross-competes with the herein-disclosed antibody or antigen-binding fragment thereof.

In one aspect, the disclosure relates to an antibody-drug conjugate containing the herein-disclosed antibody or antigen-binding fragment thereof covalently bound to a therapeutic agent.

In one aspect, the disclosure relates to a pharmaceutical composition containing the herein-disclosed antibody or antigen-binding fragment thereof, or the herein-disclosed antibody-drug conjugate, and a pharmaceutically acceptable carrier.

In one aspect, the disclosure relates to a nucleic acid encoding the herein-disclosed antibody or antigen-binding fragment thereof. In one aspect, the disclosure relates to a vector containing such a nucleic acid. In one aspect, the disclosure relates to a cell containing such a vector.

In one aspect, the disclosure relates to a method of producing an antibody or an antigen-binding fragment thereof, the method including culturing the cell containing a nucleic acid encoding the herein-disclosed antibody or antigen-binding fragment thereof under conditions sufficient for the cell to produce the antibody or the antigen-binding fragment thereof; and collecting the antibody or the antigen-binding fragment thereof produced by the cell.

In one aspect, the disclosure relates to an engineered receptor containing the herein-disclosed antigen-binding fragment thereof.

In some embodiments, the engineered receptor further has a transmembrane domain, and an intracellular signaling domain.

In some embodiments, the engineered receptor is a chimeric antigen receptor (“CAR”).

In some embodiments, the engineered receptor further has a hinge domain.

In some embodiments, the transmembrane domain has a transmembrane domain of CD4, CD8, and/or CD28, or a portion thereof.

In some embodiments, the intracellular signaling domain has a primary intracellular signaling sequence of an immune effector cell.

In some embodiments, the intracellular signaling domain is or has a functional signaling domain of CD3 zeta.

In some embodiments, the intracellular signaling domain further has a costimulatory signaling domain.

In some embodiments, the costimulatory signaling domain has a functional signaling domain from a protein selected from the group consisting of a MHC class I molecule, a TNF receptor protein, an Immunoglobulin-like protein, a cytokine receptor, an integrin, a signaling lymphocytic activation molecule (SLAM protein), an activating NK cell receptor, BTLA, a Toll ligand receptor, OX40, CD2, CD7, CD27, CD28, CD30, CD40, CDS, ICAM-1, LFA-1, CD11a/CD18, 4-1BB (CD137), B7-H3, CDS, ICAM-1, ICOS (CD278), GITR, BAFFR, LIGHT, HVEM (LIGHTR), KIRDS2, SLAMF7, NKp80 (KLRF1), NKp44, NKp30, NKp46, CD19, CD4, CD8alpha, CD8beta, IL2R beta, IL2R gamma, IL7R alpha, ITGA4, VLA1, CD49a, ITGA4, IA4, CD49D, ITGA6, VLA-6, CD49f, ITGAD, CD11d, ITGAE, CD103, ITGAL, CD11a, LFA-1, ITGAM, CD 11b, ITGAX, CD 11c, ITGB1, CD29, ITGB2, CD18, LFA-1, ITGB7, NKG2D, NKG2C, TNFR2, TRANCE/RANKL, DNAM1 (CD226), SLAMF4 (CD244, 2B4), CD84, CD96 (Tactile), CEACAMI, CRTAM, Ly9 (CD229), CD160 (BY55), PSGL1, CD100 (SEMA4D), CD69, SLAMF6 (NTB-A, Lyl08), SLAM (SLAMF1, CD150, IPO-3), BLAME (SLAMF8), SELPLG (CD162), LTBR, LAT, GADS, SLP-76, PAG/Cbp, CD19a, and a CD83 ligand.

In some embodiments, the costimulatory signaling domain has an intracellular signaling domain of 4-1BB and/or CD28.

In some embodiments, the engineered receptor has a signal peptide. In some embodiments, the signal peptide is at least 80%, 85%, 90%, 95% or 100% identical to SEQ ID NO: 47.

In some embodiments, the engineered receptor has an amino acid sequence set forth in any one of SEQ ID NOs: 54-61 and 97-131, or an amino acid sequence that is at least 90%, 91%, 92%, 93%, 94%, 95%, 96%, 97%, 98%, 99% or 100% identical to any one of SEQ ID NOs: 54-61 and 97-131. In some embodiments, the amino acid sequence is identical to any one of SEQ ID NOs: 54-61 and 97-131.

In some embodiments, the engineered receptor is a chimeric T cell receptor (“cTCR”).

In some embodiments, the transmembrane domain of the engineered receptor is derived from the transmembrane domain of a TCR subunit selected from the group consisting of TCRα, TCRβ, TCRγ, TCRδ, CD3γ, CD3ε, and CD3δ. In some embodiments, the transmembrane domain is derived from the transmembrane domain of CD3ε.

In some embodiments, the intracellular signaling domain of the engineered receptor is derived from the intracellular signaling domain of a TCR subunit selected from the group consisting of TCRα, TCRβ, TCRγ, TCRδ, CD3γ, CD3ε, and CD3δ. In some embodiments, the intracellular signaling domain is derived from the intracellular signaling domain of CD3ε.

In some embodiments, the engineered receptor further has at least a portion of an extracellular domain of a TCR subunit.

In some embodiments, the antigen binding fragment of the engineered receptor is fused to the N-terminus of CD3ε(“eTCR”).

In one aspect, the disclosure relates to a polynucleotide encoding the herein-disclosed engineered receptor. In one aspect, the disclosure relates to a vector containing such polynucleotide. In some embodiments, the vector is a viral vector.

In one aspect, the disclosure relates to an engineered cell expressing the herein-disclosed engineered receptor.

In some embodiments, the engineered cell is an immune cell.

In some embodiments, the immune cell is an NK cell or a T cell.

In some embodiments, the engineered cell is a T cell.

In some embodiments, the T cell is selected from the group consisting of cytotoxic T cell, a helper T cell, a natural killer T (NK-T) cell, and a TST cell.

In one aspect, the disclosure relates to a method for producing an engineered cell, including introducing a vector of containing a polynucleotide encoding the herein-disclosed engineered receptor into a cell in vitro or ex vivo.

In some embodiments, the vector is a viral vector and the introducing is carried out by transduction.

In one aspect, the disclosure relates to a method of treating cancer in a subject, including administering an effective amount of the herein-disclosed antibody or antigen-binding fragment thereof, the herein-disclosed antibody-drug conjugate, the herein-disclosed pharmaceutical composition, or the herein-disclosed engineered cell to the subject.

In some embodiments, the cancer is liver cancer, lung cancer, or esophageal cancer.

In one aspect, the disclosure provides an antibody or antigen-binding fragment thereof that binds to GPC3, containing: a heavy chain variable region (VH) having complementarity determining regions (CDRs) 1, 2, and 3, wherein the VH CDR1 region has an amino acid sequence that is at least 80% or 90% identical to a selected VH CDR1 amino acid sequence, the VH CDR2 region has an amino acid sequence that is at least 80% or 90% identical to a selected VH CDR2 amino acid sequence, and the VH CDR3 region has an amino acid sequence that is at least 80% or 90% identical to a selected VH CDR3 amino acid sequence; and a light chain variable region (VL) having CDRs 1, 2, and 3, wherein the VL CDR1 region has an amino acid sequence that is at least 80% or 90% identical to a selected VL CDR1 amino acid sequence, the VL CDR2 region has an amino acid sequence that is at least 80% or 90% identical to a selected VL CDR2 amino acid sequence, and the VL CDR3 region has an amino acid sequence that is at least 80% or 90% identical to a selected VL CDR3 amino acid sequence, wherein the selected VH CDRs 1, 2, and 3 amino acid sequences are one of the following:
(1) the selected VH CDRs 1, 2, 3 amino acid sequences are set forth in SEQ ID NOs: 20, 22 and 29 respectively;
(2) the selected VH CDRs 1, 2, 3 amino acid sequences are set forth in SEQ ID NOs: 21, 22 and 29 respectively;
(3) the selected VH CDRs 1, 2, 3 amino acid sequences are set forth in SEQ ID NOs: 20, 23 and 29 respectively;
(4) the selected VH CDRs 1, 2, 3 amino acid sequences are set forth in SEQ ID NOs: 21, 23 and 29 respectively;
(5) the selected VH CDRs 1, 2, 3 amino acid sequences are set forth in SEQ ID NOs: 21, 24 and 29 respectively;
(6) the selected VH CDRs 1, 2, 3 amino acid sequences are set forth in SEQ ID NOs: 21, 25 and 29 respectively;
(7) the selected VH CDRs 1, 2, 3 amino acid sequences are set forth in SEQ ID NOs: 21, 26 and 29 respectively;
(8) the selected VH CDRs 1, 2, 3 amino acid sequences are set forth in SEQ ID NOs: 21, 27 and 29 respectively;
(9) the selected VH CDRs 1, 2, 3 amino acid sequences are set forth in SEQ ID NOs: 21, 28 and 29 respectively;
wherein the selected VL CDRs, 1, 2, and 3 amino acid sequences are one of the following:
(1) the selected VL CDRs 1, 2, 3 amino acid sequences are set forth in SEQ ID NOs: 30, 32 and 34 respectively;
(2) the selected VL CDRs 1, 2, 3 amino acid sequences are set forth in SEQ ID NOs: 30, 33 and 34 respectively;
(3) the selected VL CDRs 1, 2, 3 amino acid sequences are set forth in SEQ ID NOs: 31, 33 and 34 respectively;
(4) the selected VL CDRs 1, 2, 3 amino acid sequences are set forth in SEQ ID NOs: 30, 33 and 35 respectively;
(5) the selected VL CDRs 1, 2, 3 amino acid sequences are set forth in SEQ ID NOs: 30, 33 and 36 respectively;
(6) the selected VL CDRs 1, 2, 3 amino acid sequences are set forth in SEQ ID NOs: 30, 33 and 37 respectively;
(7) the selected VL CDRs 1, 2, 3 amino acid sequences are set forth in SEQ ID NOs: 30, 32 and 35 respectively.

As used herein, the term “antibody” refers to any antigen-binding molecule that contains at least one (e.g., one, two, three, four, five, or six) complementary determining region (CDR) (e.g., any of the three CDRs from an immunoglobulin light chain or any of the three CDRs from an immunoglobulin heavy chain) and is capable of specifically binding to an epitope in an antigen. Non-limiting examples of antibodies include: monoclonal antibodies, polyclonal antibodies, multi-specific antibodies (e.g., bi-specific antibodies), single-chain antibodies (scFv), single variable domain (VHH) antibodies, chimeric antibodies, human antibodies, and humanized antibodies. In some embodiments, an antibody can contain an Fc region of a human antibody. The term antibody also includes derivatives, e.g., multi-specific antibodies, bi-specific antibodies, single-chain antibodies, diabodies, and linear antibodies formed from these antibodies or antibody fragments.

As used herein, the term “antigen-binding fragment” refers to a portion of a full-length antibody, wherein the portion of the antibody is capable of specifically binding to an antigen. In some embodiments, the antigen-binding fragment contains at least one variable domain (e.g., a variable domain of a heavy chain, a variable domain of light chain or a V_{H}H). Non-limiting examples of antibody fragments include, e.g., Fab, Fab′, F(ab′)₂, and Fv fragments, scFv, and V_{H}H.

As used herein, the terms “subject” and “patient” are used interchangeably throughout the specification and describe an animal, human or non-human, to whom treatment according to the methods of the present disclosure is provided. Veterinary and non-veterinary applications are contemplated in the present disclosure. Human patients can be adult humans or juvenile humans (e.g., humans below the age of 18 years old). In addition to humans, patients include but are not limited to mice, rats, hamsters, guinea-pigs, rabbits, ferrets, cats, dogs, and primates. Included are, for example, non-human primates (e.g., monkey, chimpanzee, gorilla, and the like), rodents (e.g., rats, mice, gerbils, hamsters, ferrets, rabbits), lagomorphs, swine (e.g., pig, miniature pig), equine, canine, feline, bovine, and other domestic, farm, and zoo animals.

As used herein, when referring to an antibody or an antigen-binding fragment, the phrases “specifically binding” and “specifically binds” mean that the antibody or an antigen-binding fragment interacts with its target molecule preferably to other molecules, because the interaction is dependent upon the presence of a particular structure (i.e., the antigenic determinant or epitope) on the target molecule; in other words, the reagent is recognizing and binding to molecules that include a specific structure rather than to all molecules in general. An antibody that specifically binds to the target molecule may be referred to as a target-specific antibody. For example, an antibody that specifically binds to GPC3 may be referred to as a GPC3 antibody, a GPC3-specific antibody or an anti-GPC3 antibody.

As used herein, the term “bispecific antibody” refers to an antibody that binds to two different epitopes. The epitopes can be on the same antigen or on different antigens.

As used herein, the term “trispecific antibody” refers to an antibody that binds to three different epitopes. The epitopes can be on the same antigen or on different antigens.

As used herein, the term “multispecific antibody” refers to an antibody that binds to two or more different epitopes. The epitopes can be on the same antigen or on different antigens. A multispecific antibody can be e.g., a bispecific antibody or a trispecific antibody. In some embodiments, the multispecific antibody binds to two, three, four, five, or six different epitopes.

As used herein, a “V_{H}H” refers to the variable domain of a heavy chain only antibody. In some embodiments, the V_{H}H is a humanized V_{H}H.

As used herein, a “chimeric antigen receptor” or “CAR” refers to a fusion protein comprising an extracellular domain capable of binding to an antigen, and an intracellular region comprising one or more intracellular signaling domains derived from signal transducing proteins. These intracellular signaling domains are typically different from the polypeptide from which the extracellular domain is derived. The extracellular domain can be any proteinaceous molecule or part thereof that can specifically bind to a predetermined antigen. In some embodiments, the extracellular domain comprises an antibody or antigen binding fragment thereof. In some embodiments, the intracellular signaling domain can be any oligopeptide or polypeptide domain known to function to transmit a signal causing activation or inhibition of a biological process in a cell, for example, activation of an immune cell such as a T cell or a NK cell.

As used herein, a “tandem CAR” refers to a CAR comprising two or more extracellular domain capable of binding to an antigen. In some embodiments, a tandem CAR can have 2, 3, 4, 5, 6, 7, 8, 9, or 10 extracellular domains that are capable of binding to an antigen. These antigen-binding domains can be the same or different. In some embodiments, they can bind to the same or different antigens. In some embodiments, the can bind to different epitopes on the same antigen.

Unless otherwise defined, all technical and scientific terms used herein have the same meaning as commonly understood by one of ordinary skill in the art to which this disclosure belongs. Methods and materials are described herein for use in the present disclosure; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

Other features and advantages of the disclosure will be apparent from the following detailed description and figures, and from the claims.

### DESCRIPTION OF DRAWINGS

Exemplary embodiments are illustrated in referenced figures. It is intended that the embodiments and figures disclosed herein are to be considered illustrative rather than restrictive.

FIG. 1 shows a schematic diagram of the chimeric antigen receptor (CAR). The vector construct includes sequences encoding: a signal peptide (SP), a GPC3-binding domain (GPC3-scFv), a CD8 hinge, a CD8 transmembrane domain (CD8 TM), a 4-1BB intracellular co-stimulatory domain, and a CD3ξ intracellular signaling sequence.

FIG. 2A shows the number of humanized anti-GPC3 CAR αβ T cells with an extracellular scFv on day 0, day 4 and day 6 after transduction. Un-transduced T cells (UNT) were used as control cells.

FIG. 2B shows the percentage of viable humanized anti-GPC3 CAR αβ T cells with an extracellular scFv on day 0, day 4 and day 6 after transduction. Un-transduced T cells (UNT) were used as control cells.

FIG. 3A shows calculated cytotoxicity (Cytotoxicity %) of humanized anti-GPC3 CAR αβ T cells in long-term co-culture assays with Huh7 cells. The T cells were re-challenged for a total of 8 times. Un-transduced T cells (UNT) were used as the negative control.

FIG. 3B shows the expansion fold curves of humanized anti-GPC3 CAR αβ T cells in long-term co-culture assays with Huh7 cells. The T cells were re-challenged for a total of 8 times. The curves reflected the calculated T cell proliferation.

FIG. 4A shows the number of humanized anti-GPC3 CAR γδ T cells with an extracellular scFv on day 0, and day 9 after transduction. Un-transduced T cells (UNT) were used as control cells.

FIG. 4B shows the percentage of viable humanized anti-GPC3 CAR γδ T cells with an extracellular scFv on day 0, and day 9 after transduction. Un-transduced T cells (UNT) were used as control cells.

FIG. 5A shows calculated cytotoxicity (Cytotoxicity %) of humanized anti-GPC3 CAR γδ T cells in long-term co-culture assays with Huh7 cells. The T cells were re-challenged for a total of 5 times. Un-transduced T cells (UNT) were used as the negative control.

FIG. 5B shows the expansion fold curves of humanized anti-GPC3 CAR γδ T cells in long-term co-culture assays with Huh7 cells. The T cells were re-challenged for a total of 5 times. The curves reflected the calculated T cell proliferation.

FIGS. 6A-6D show in vivo efficacy of humanized anti-GPC3 CAR γδ T cells in mouse Huh7 xenograft model. The curves reflected the calculated tumor volume.

FIGS. 7A, 8A and 9A show calculated cytotoxicity (Cytotoxicity %) of further humanized anti-GPC3 CAR αβ T cells in long-term co-culture assays with Huh7 cells. Un-transduced T cells (UNT) were used as the negative control.

FIGS. 7B, 8B and 9B show the expansion fold curves of further humanized anti-GPC3 CAR αβ T cells in long-term co-culture assays with Huh7 cells. The curves reflected the calculated T cell proliferation.

FIG. 10A shows calculated cytotoxicity (Cytotoxicity %) of further humanized anti-GPC3 CAR γδ T cells in long-term co-culture assays with Huh7 cells. Un-transduced T cells (UNT) were used as the negative control.

FIG. 10B shows the expansion fold curves of further humanized anti-GPC3 CAR γδ T cells in long-term co-culture assays with Huh7 cells. The curves reflected the calculated T cell proliferation.

FIG. 11 shows in vivo efficacy of further humanized anti-GPC3 CAR γδ T cells in mouse Huh7 xenograft model. The curves reflected the calculated tumor volume.

FIG. 12A shows different combinations of VH and VL.

FIG. 12B shows different combinations of VH CDRs.

FIG. 12C shows different combinations of VL CDRs.

FIG. 13 lists the sequences discussed in the disclosure.

### DETAILED DESCRIPTION

Provided herein are compositions and methods for treating diseases associated with expression of GPC3. The disclosure also relates to chimeric antigen receptor (CAR) specific to GPC3, and engineered immune effector cells (such as T cells) comprising the GPC3 CAR. The disclosure also includes methods of administering a genetically modified T cell expressing a CAR that comprises an optimized GPC3 binding domain.

### GPC3 and Cancer

Glypican 3 (GPC3) is a glycosylphosphatidylinositol-anchored cell surface protein consisting of a core protein and two heparan sulfate HS chains (Filmus J. et al., Biochem J. 1995 Oct. 15; 311(Pt 2):561-5). GPC3 is an oncofetal protein expressed in over 70% of HCC (Capurro M. et al., Gastroenterology. 2003 July; 125(1):89-97.) and other solid tumors including hepatoblastoma and lung squamous cell carcinoma (Baumhoer D. et al., Am J Clin Pathol. 2008 June; 129(6):899-906; Aviel-Ronen S. et al., Mod Pathol. 2008 July; 21(7):817-25). Its expression is not detected in nonmalignant adult tissues including normal liver (Montalbano M. et al., Oncol Rep. 2017 March; 37(3):1291-1300). Mechanistically, GPC3 can promote tumor growth by modulating the Wnt/Frizzled signaling complex on HCC cells (Li N. et al., Hepatology. 2019 October; 70(4):1231-1245). Considering the high specific expression of GPC3 in hepatocellular carcinoma, melanoma and other tumors, it is considered as a candidate target antigen for CAR T cells tumor immunotherapy.

The protein core of GPC3 consists of two subunits, where the N-terminal subunit has a size of about 40 kDa and the C-terminal subunit is about 30 kDa. Six glypicans (GPC1-6) have been identified in mammals.

Details of GPC3 can be found, e.g., in Filmus, Jorge, et al. “Identification of a new membrane-bound heparan sulphate proteoglycan.” Biochemical Journal 311.2 (1995): 561-565; Capurro M. et al. Glypican-3: a novel serum and histochemical marker for hepatocellular carcinoma. Gastroenterology. 2003 July; 125(1):89-97; Baumhoer D. et al. Glypican 3 expression in human nonneoplastic, preneoplastic, and neoplastic tissues: a tissue microarray analysis of 4,387 tissue samples. Am J Clin Pathol. 2008 June; 129(6):899-906; Aviel-Ronen S. et al. Glypican-3 is overexpressed in lung squamous cell carcinoma, but not in adenocarcinoma. Mod Pathol. 2008 July; 21(7):817-25; Montalbano M. et al. Biology and function of glypican-3 as a candidate for early cancerous transformation of hepatocytes in hepatocellular carcinoma. Oncol Rep. 2017 March; 37(3):1291-1300; and Li N. et al. A Frizzled-Like Cysteine-Rich Domain in Glypican-3 Mediates Wnt Binding and Regulates Hepatocellular Carcinoma Tumor Growth in Mice. Hepatology. 2019 October; 70(4):1231-1245; each of which is incorporated herein by reference in its entirety.

The present disclosure provides antibodies and antigen-binding fragment thereof (e.g., single chain variable fragments) targeting GPC3, and chimeric antigen receptors targeting GPC3.

### Antibodies and Antigen Binding Fragments

The present disclosure provides antibodies and antigen-binding fragments thereof that bind to GPC3 (e.g., human GPC3 or extracellular region of GPC3).

In general, antibodies (also called immunoglobulins) are made up of two classes of polypeptide chains, light chains and heavy chains. A non-limiting antibody of the present disclosure can be an intact, four immunoglobulin chain antibody comprising two heavy chains and two light chains. The heavy chain of the antibody can be of any isotype including IgM, IgG, IgE, IgA, or IgD or sub-isotype including IgG1, IgG2, IgG2a, IgG2b, IgG3, IgG4, IgE1, IgE2, etc. The light chain can be a kappa light chain or a lambda light chain. An antibody can have two identical copies of a light chain and two identical copies of a heavy chain. The heavy chains, which each contain one variable domain (or variable region, VH) and multiple constant domains (or constant regions), bind to one another via disulfide bonding within their constant domains to form the “stem” of the antibody. The light chains, which each contain one variable domain (or variable region, VL) and one constant domain (or constant region), each bind to one heavy chain via disulfide binding. The variable region of each light chain is aligned with the variable region of the heavy chain to which it is bound. The variable regions of both the light chains and heavy chains contain three hypervariable regions sandwiched between more conserved framework regions (FR).

These hypervariable regions, known as the complementary determining regions (CDRs), form loops that comprise the principle antigen binding surface of the antibody. The four framework regions largely adopt a beta-sheet conformation and the CDRs form loops connecting, and in some cases forming part of, the beta-sheet structure. The CDRs in each chain are held in close proximity by the framework regions and, with the CDRs from the other chain, contribute to the formation of the antigen-binding region.

Methods for identifying the CDR regions of an antibody by analyzing the amino acid sequence of the antibody are well known, and a number of definitions of the CDRs are commonly used. The Kabat definition is based on sequence variability, and the AbM definition is based on the location of the structural loop regions. These methods and definitions are described in, e.g., Martin, Antibody engineering, Springer Berlin Heidelberg, 2001. 422-439; Abhinandan, et al., Molecular immunology 45.14 (2008): 3832-3839; Wu, T. T. and Kabat, E. A. (1970) J. Exp. Med. 132: 211-250; Martin et al., Methods Enzymol. 203:121-53 (1991); Morea et al., Biophys Chem. 68(1-3):9-16 (October 1997); Morea et al., J Mol Biol. 275(2):269-94 (January 1998); Chothia et al., Nature 342(6252):877-83 (December 1989); Ponomarenko and Bourne, BMC Structural Biology 7:64 (2007); each of which is incorporated herein by reference in its entirety. In some embodiments, the Kabat definition is used. In some embodiments, the AbM definition is used. In some embodiments, a combination of Kabat and AbM, and/or some other definitions that are well known in the art are used.

The CDRs are important for recognizing an epitope of an antigen. As used herein, an “epitope” is the smallest portion of a target molecule capable of being specifically bound by the antigen binding domain of an antibody. The minimal size of an epitope may be about three, four, five, six, or seven amino acids, but these amino acids need not be in a consecutive linear sequence of the antigen's primary structure, as the epitope may depend on an antigen's three-dimensional configuration based on the antigen's secondary and tertiary structure.

In some embodiments, the antibody is an intact immunoglobulin molecule (e.g., IgG1, IgG2a, IgG2b, IgG3, IgM, IgD, IgE, IgA). The IgG subclasses (IgG1, IgG2, IgG3, and IgG4) are highly conserved, differ in their constant region, particularly in their hinges and upper CH2 domains. The sequences and differences of the IgG subclasses are known in the art, and are described, e.g., in Vidarsson, et al, Frontiers in immunology 5 (2014); Irani, et al., Molecular immunology 67.2 (2015): 171-182; Shakib, Farouk, ed. The human IgG subclasses: molecular analysis of structure, function and regulation. Elsevier, 2016; each of which is incorporated herein by reference in its entirety.

The antibody can also be an immunoglobulin molecule that is derived from any species (e.g., human, rodent, mouse, camelid). Antibodies disclosed herein also include, but are not limited to, polyclonal, monoclonal, monospecific, polyspecific antibodies, and chimeric antibodies that include an immunoglobulin binding domain fused to another polypeptide. The term “antigen binding domain” or “antigen binding fragment” is a portion of an antibody that retains specific binding activity of the intact antibody, i.e., any portion of an antibody that is capable of specific binding to an epitope on the intact antibody's target molecule. It includes, e.g., Fab, Fab′, F(ab′)₂, and variants of these fragments. Thus, in some embodiments, an antibody or an antigen binding fragment thereof can be, e.g., a scFv, a Fv, a Fd, a dAb, a bispecific antibody, a bispecific scFv, a diabody, a linear antibody, a single-chain antibody molecule, a multi-specific antibody formed from antibody fragments, and any polypeptide that includes a binding domain which is, or is homologous to, an antibody binding domain. Non-limiting examples of antigen binding domains include, e.g., the heavy chain and/or light chain CDRs of an intact antibody, the heavy and/or light chain variable regions of an intact antibody, full length heavy or light chains of an intact antibody, or an individual CDR from either the heavy chain or the light chain of an intact antibody.

In some embodiments, the antigen binding fragment can form a part of a chimeric antigen receptor (CAR). In some embodiments, the chimeric antigen receptor are fusions of single-chain variable fragments (scFv) as described herein, fused to CD3-zeta transmembrane and endodomain. In some embodiments, the chimeric antigen receptor also comprises intracellular signaling domains from various costimulatory protein receptors (e.g., CD28, 41BB, ICOS). In some embodiments, the chimeric antigen receptor comprises multiple signaling domains, e.g., CD3z-CD28-41BB or CD3z-CD28-OX40, to increase potency. Thus, in one aspect, the disclosure further provides cells (e.g., T cells) that express the chimeric antigen receptors as described herein. In some embodiments, the scFv has one heavy chain variable domain, and one light chain variable domain.

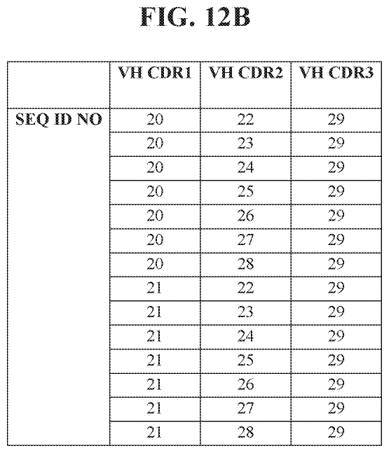

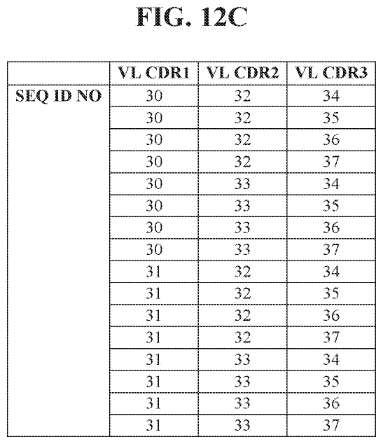

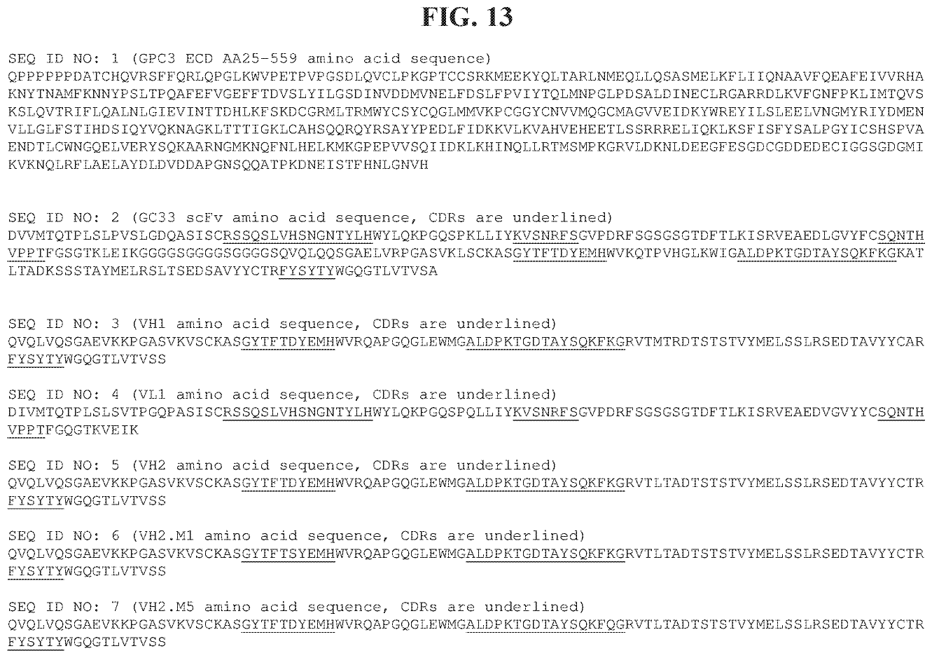

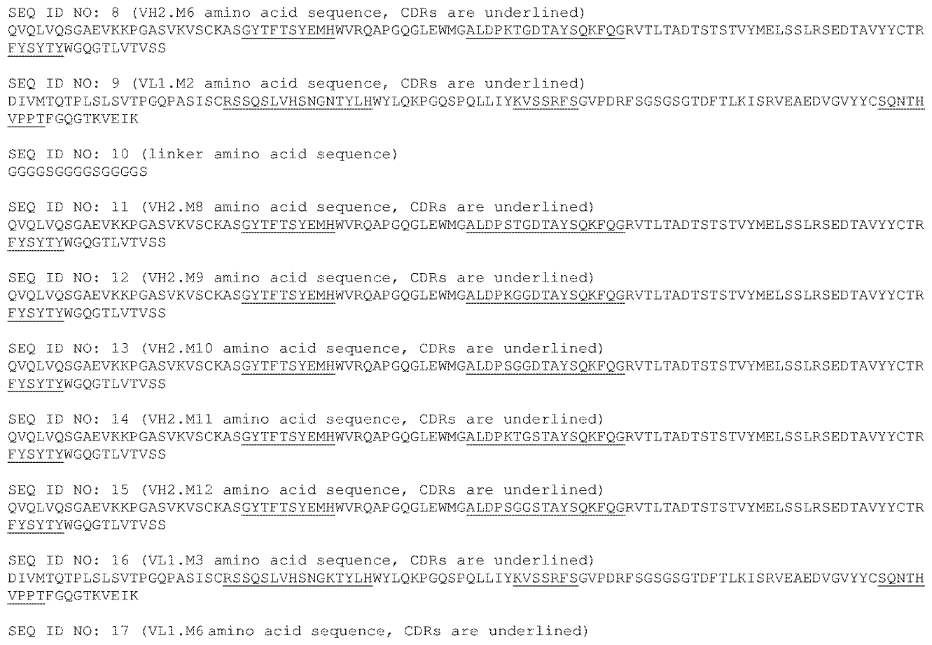

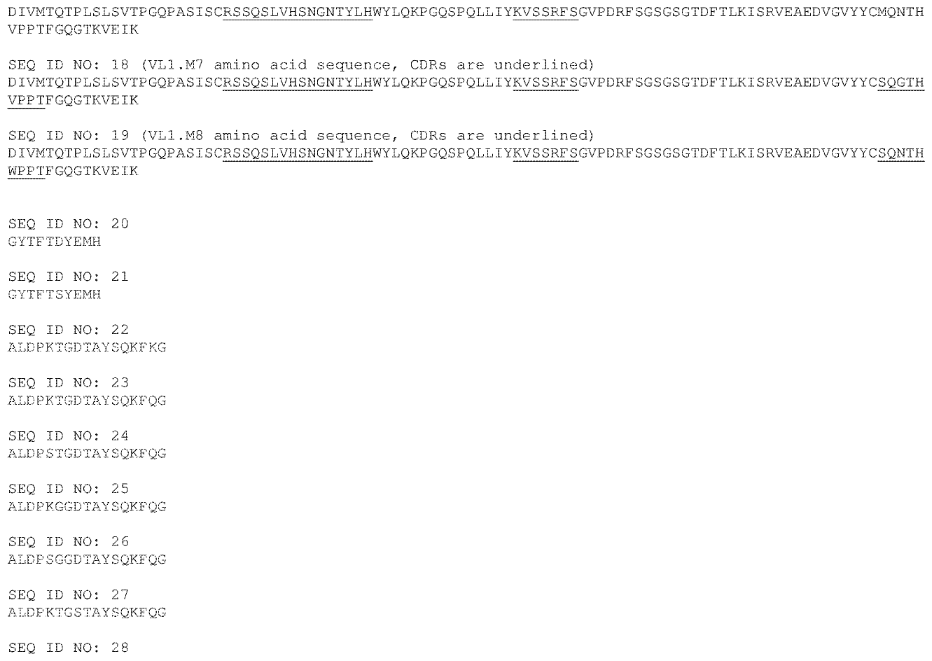

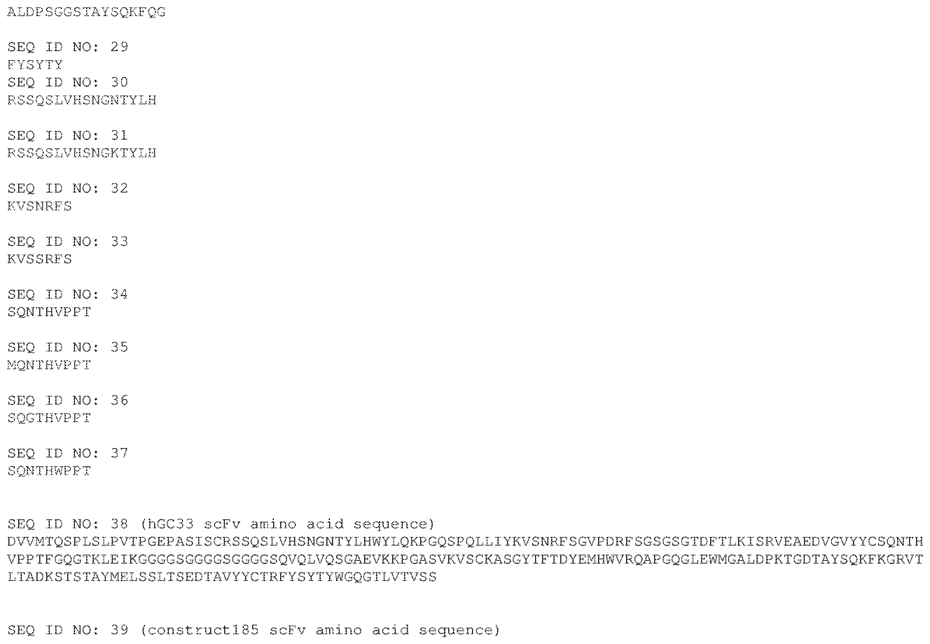

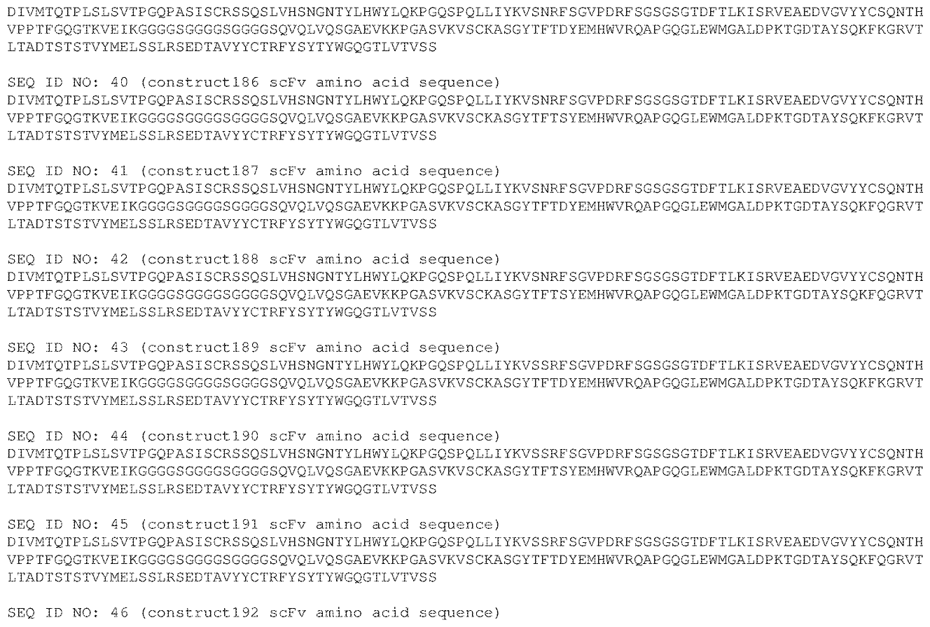

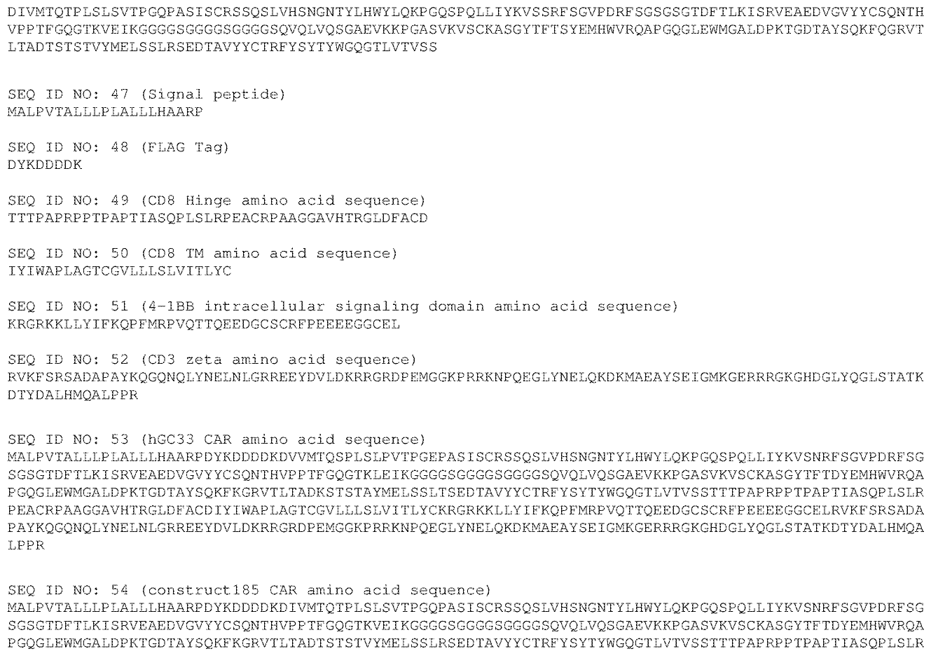

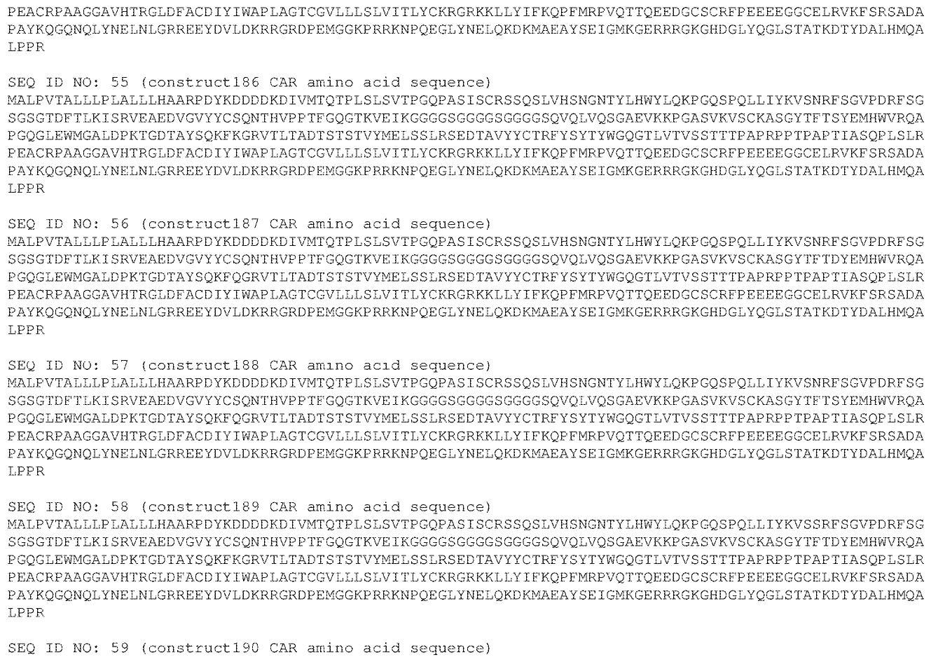

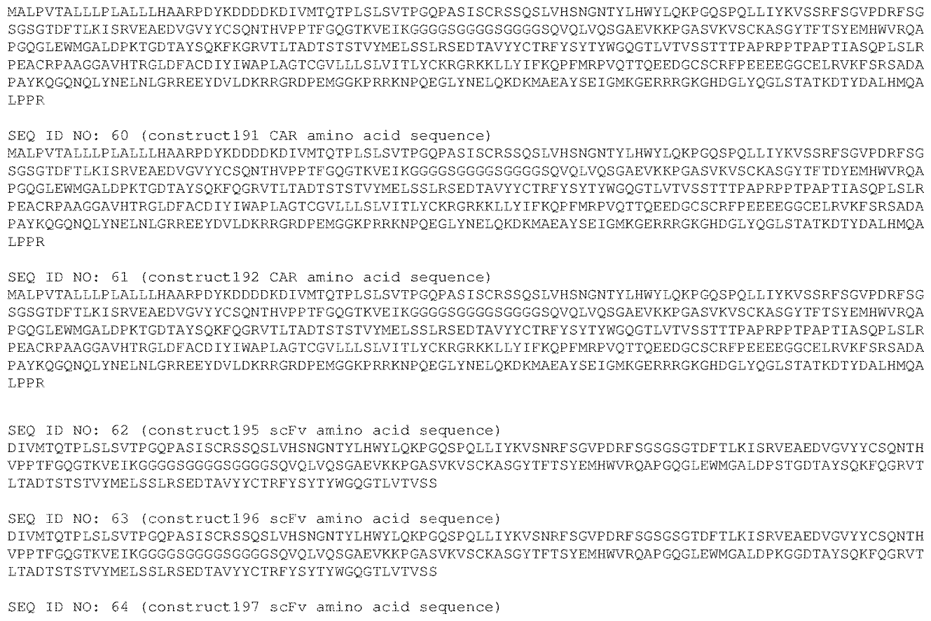

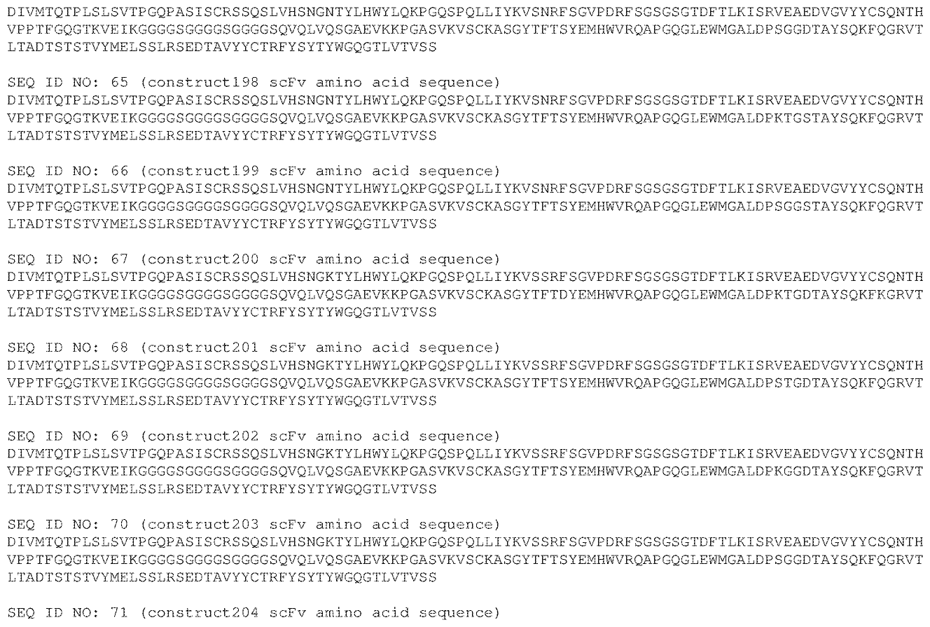

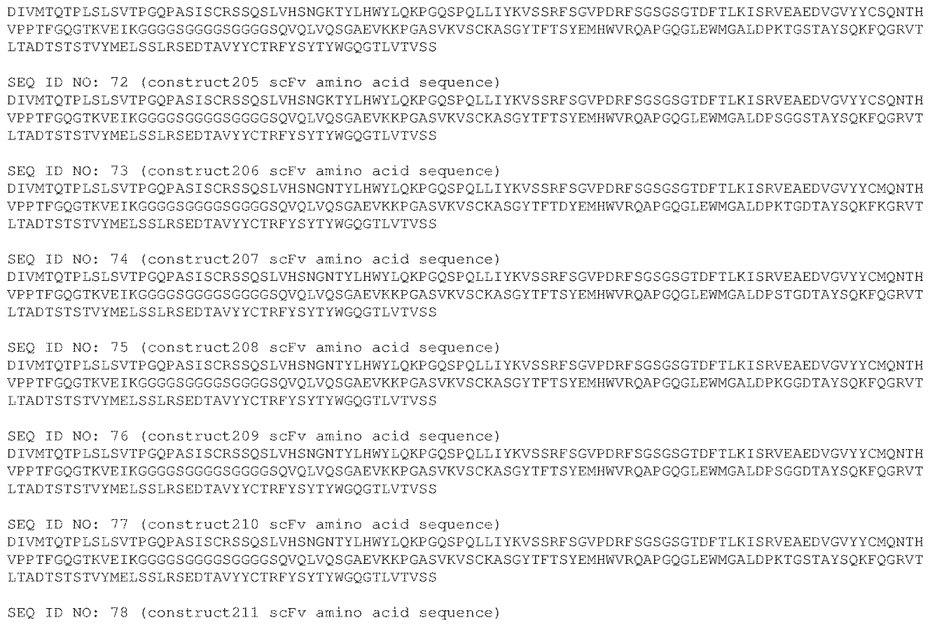

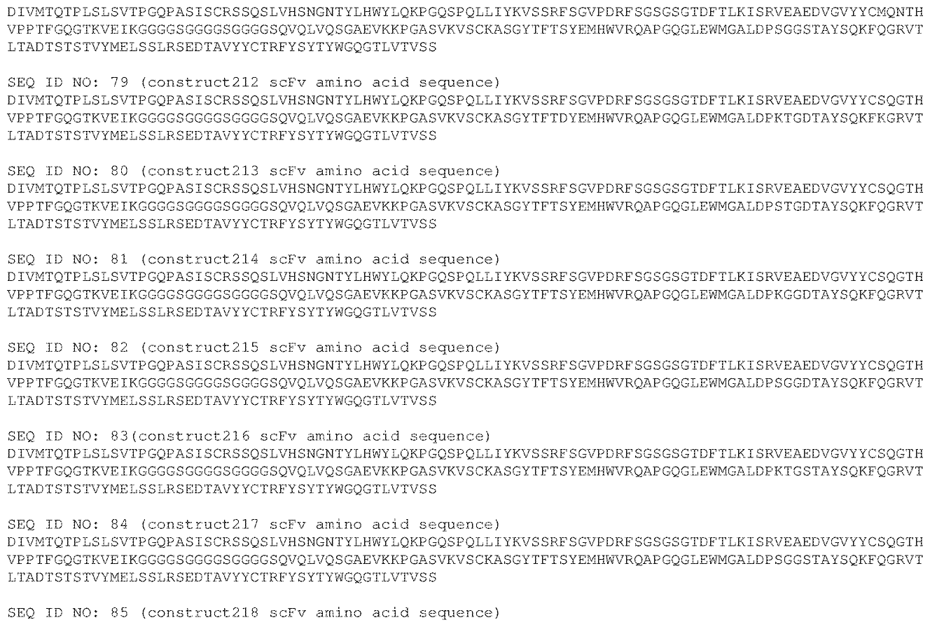

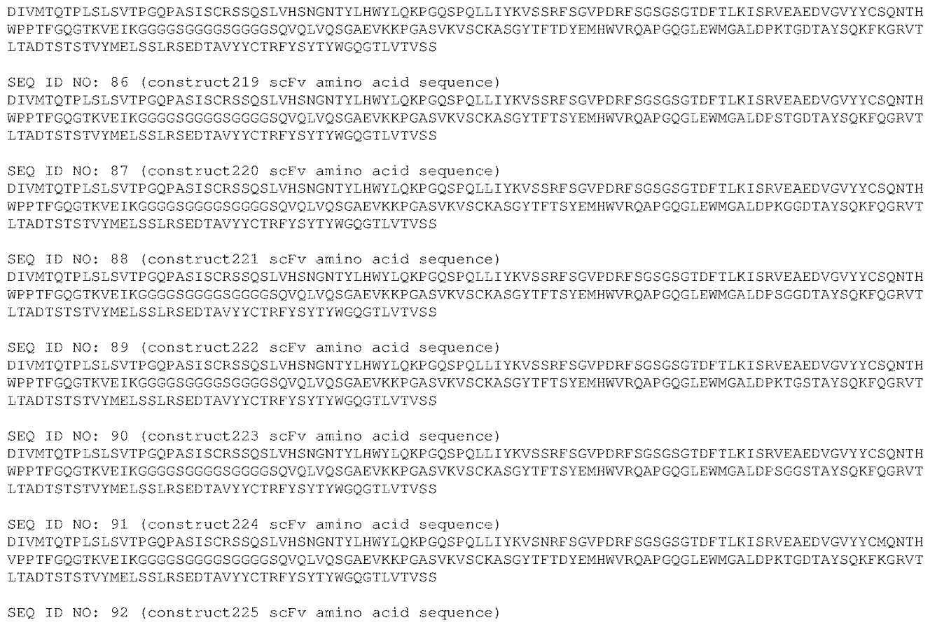

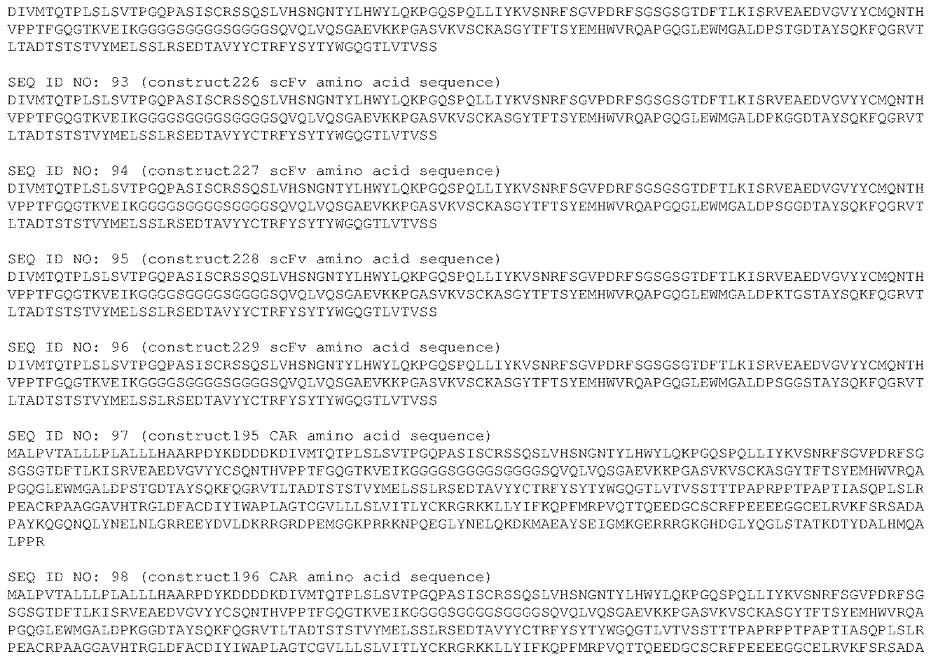

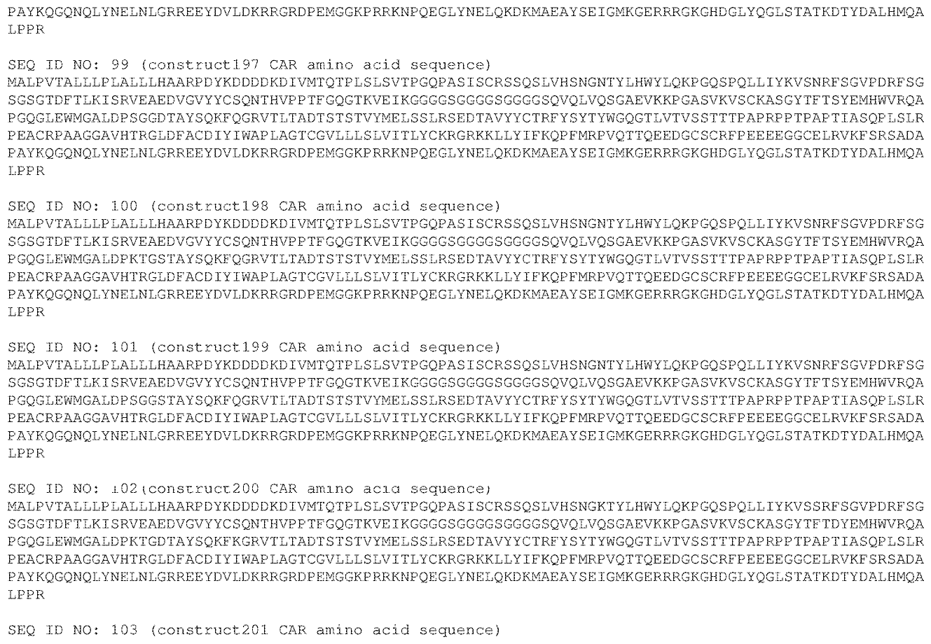

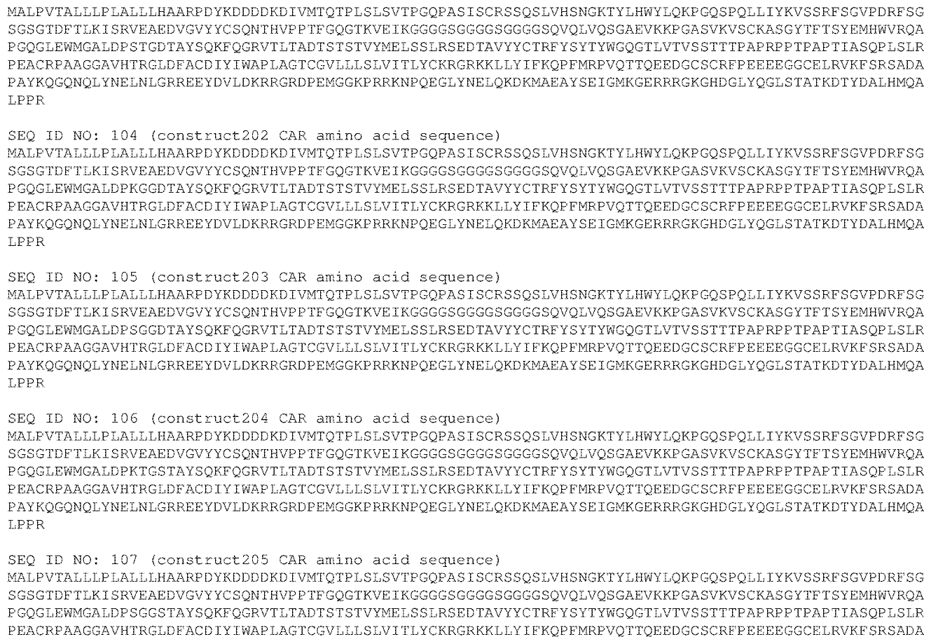

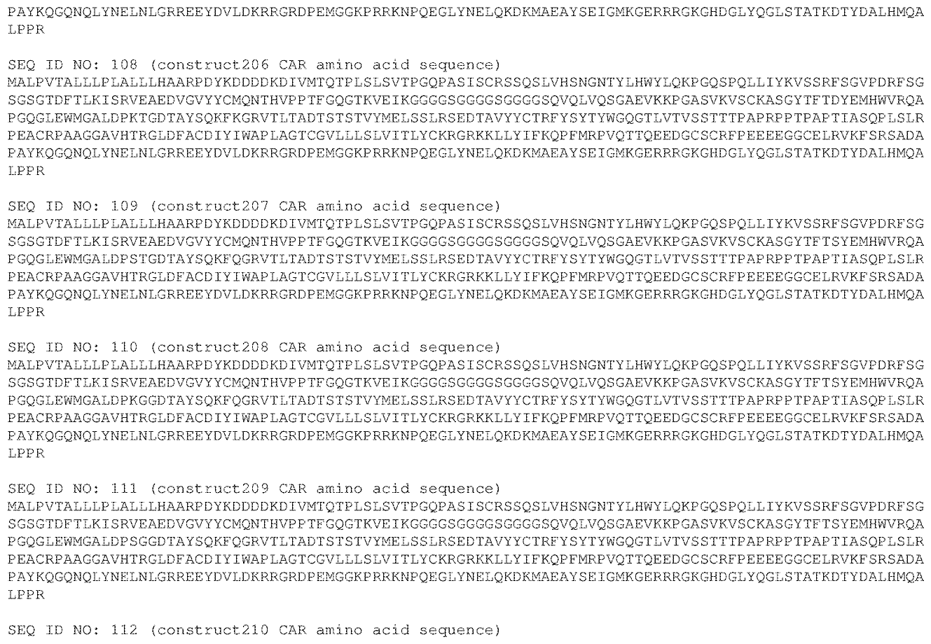

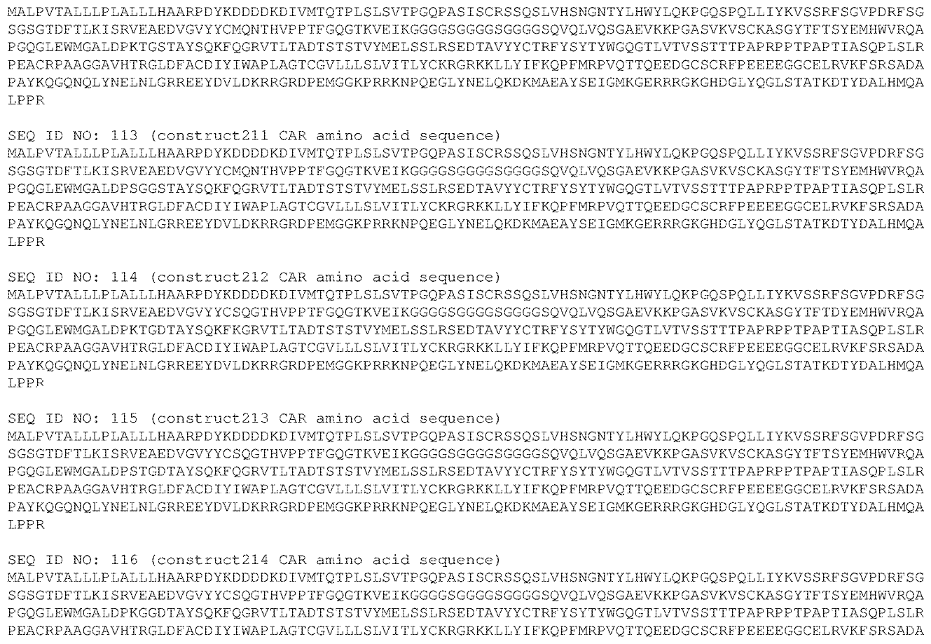

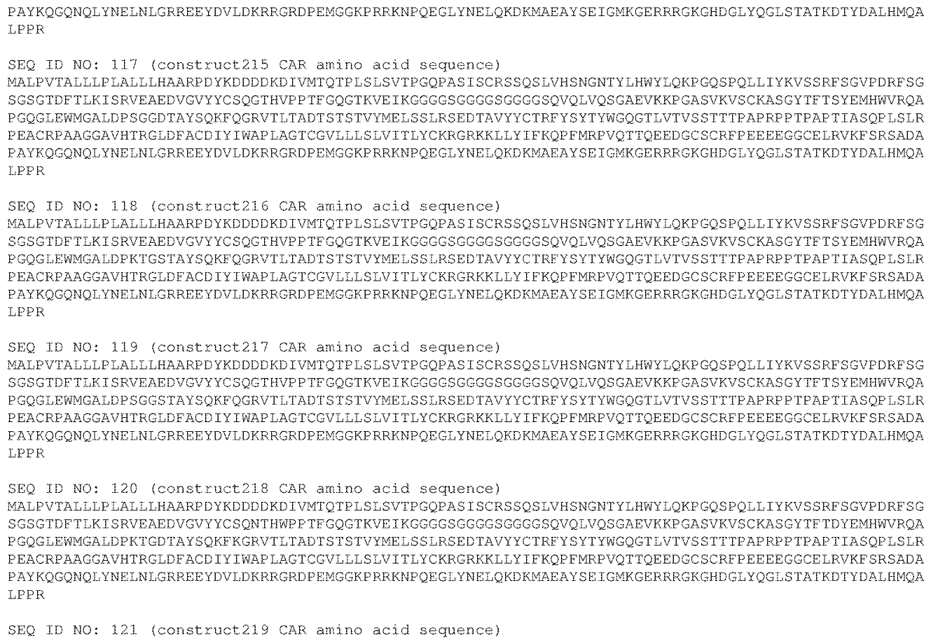

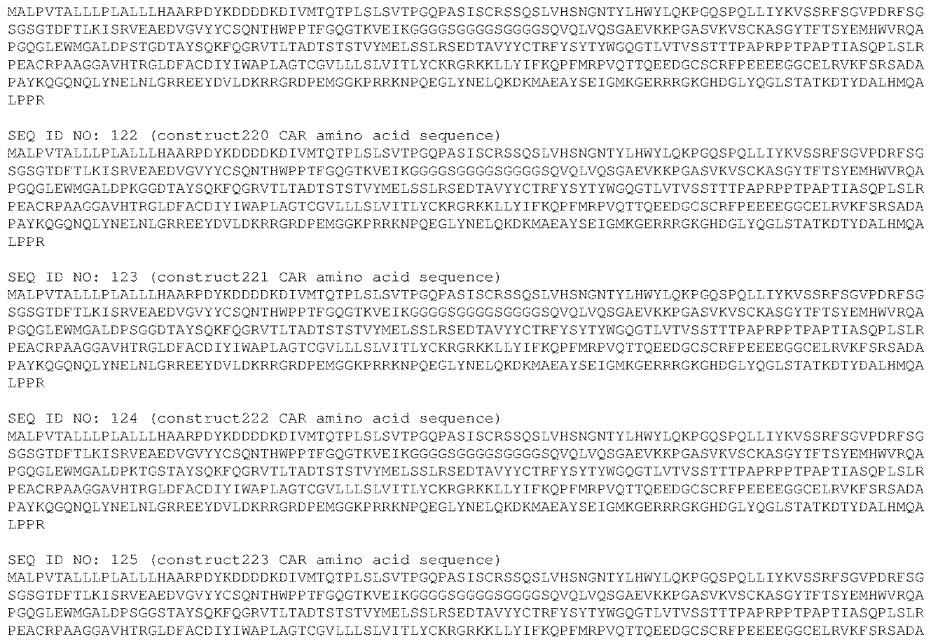

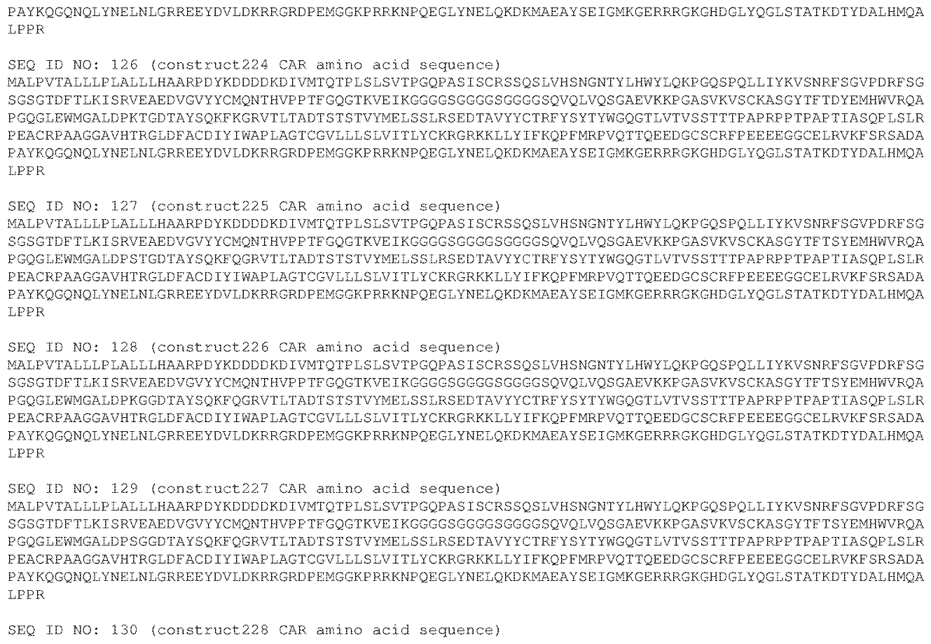

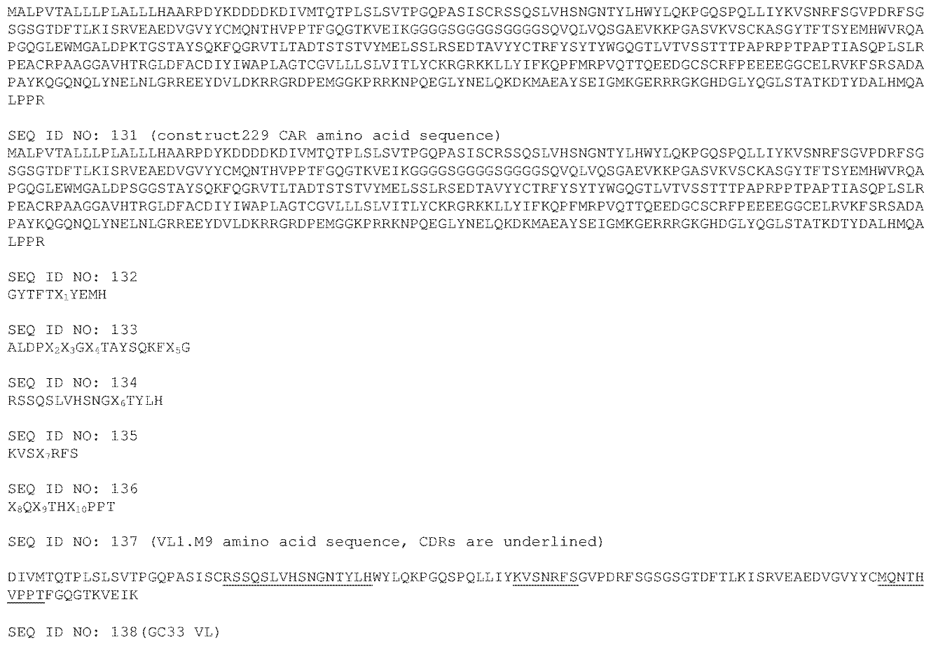

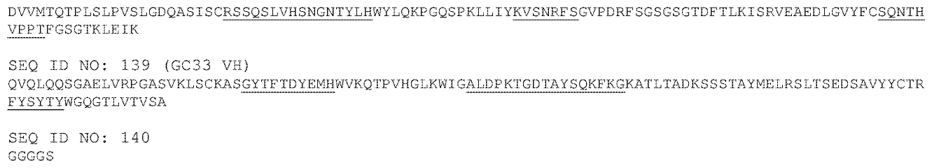

The disclosure provides antibodies and antigen-binding fragments thereof that specifically bind GPC3. The disclosure provides e.g., antibodies and antigen-binding fragments thereof, the chimeric antibodies thereof, and the humanized antibodies thereof (e.g., antibodies as shown in Tables 1 and 5, or antibodies having the combination of VH and VL as shown in FIG. 12A).

In some embodiments, the antibody or antigen-binding fragment thereof that binds to GPC3 comprises: a heavy chain variable region (VH) comprising VH complementarity determining regions (CDRs) 1, 2, and 3; and a light chain variable region (VL) comprising VL CDRs 1, 2, and 3, wherein: (a) the VH CDR1 comprises GYTFTX₁YEMH (SEQ ID NO: 132); (b) the VH CDR2 comprises ALDPX₂×₃GX₄TAYSQKFX₅G (SEQ ID NO: 133); (c) the VH CDR3 comprises FYSYTY(SEQ ID NO: 29); (d) the VL CDR1 comprises RSSQSLVHSNGX₆TYLH(SEQ ID NO: 134); (e) the VL CDR2 comprises KVSX₇RFS(SEQ ID NO: 135); and (f) the VL CDR3 comprises X₈QX₉THX₁₀PPT(SEQ ID NO: 136); and wherein: X₁ is S or D; X₂ is S or K; X₃ is G or T; X₄ is S or D; X₅ is Q or K; X₆ is N or K; X₇ is N or S; X₅ is S or M; X₉ is N or G; and X₁₀ is W or V.

In some embodiments, (a) the VH CDR1 amino acid sequence is selected from the group consisting of SEQ ID NOs: 20 and 21; (b) the VH CDR2 amino acid sequence is selected from the group consisting of SEQ ID NOs: 22-28; (c) the VH CDR3 amino acid sequence is SEQ ID NOs: 29; (d) the VL CDR1 amino acid sequence is selected from the group consisting of SEQ ID NOs: 30 and 31; (e) the VL CDR2 amino acid sequence is selected from the group consisting of SEQ ID NOs: 32 and 33; and (f) the VL CDR3 amino acid sequence is selected from the group consisting of SEQ ID NOs: 34-37. In some embodiments, the antibody or antigen-binding fragment is not an antibody or antigen-binding fragment containing VH CDRs 1, 2, 3 having amino acid sequences as set forth in SEQ ID NOs: 20, 22 and 29 respectively; and VL CDRs 1, 2, 3 having amino acid sequences as set forth in SEQ ID NOs: 30, 32 and 34 respectively. In some embodiments, VH CDR1 is not SEQ ID NO: 20. In some embodiments, VH CDR2 is not SEQ ID NO: 22. In some embodiments, VH CDR3 is not SEQ ID NO: 29. In some embodiments, VL CDR1 is not SEQ ID NO: 30. In some embodiments, VL CDR2 is not SEQ ID NO: 32. In some embodiments, VL CDR3 is not SEQ ID NO: 34. In some embodiments, the VH is not SEQ ID NO: 139. In some embodiments, the VL is not SEQ ID NO: 138.

In some embodiments, the present disclosure provides antibodies and antigen-binding fragments thereof with VH and VL CDRs that are derived from VH1-VL1, VH2-VL1, VH2.M1-VL1, VH2.M5-VL1, VH2.M6-VL1, VH2.M8-VL1, VH2.M9-VL1, VH2.M10-VL1, VH2.M11-VL1, VH2.M12-VL1, VH1-VL1.M2, VH2-VL1.M2, VH2.M1-VL1.M2, VH2.M5-VL1.M2, VH2.M6-VL1.M2, VH2.M8-VL1.M2, VH2.M9-VL1.M2, VH2.M10-VL1.M2, VH2.M11-VL1.M2, VH2.M12-VL1.M2, VH1-VL1.M3, VH2-VL1.M3, VH2.M1-VL1.M3, VH2.M5-VL1.M3, VH2.M6-VL1.M3, VH2.M8-VL1.M3, VH2.M9-VL1.M3, VH2.M10-VL1.M3, VH2.M11-VL1.M3, VH2.M12-VL1.M3, VH1-VL1.M6, VH2-VL1.M6, VH2.M1-VL1.M6, VH2.M5-VL1.M6, VH2.M6-VL1.M6, VH2.M8-VL1.M6, VH2.M9-VL1.M6, VH2.M10-VL1.M6, VH2.M11-VL1.M6, VH2.M12-VL1.M6, VH1-VL1.M7, VH2-VL1.M7, VH2.M1-VL1.M7, VH2.M5-VL1.M7, VH2.M6-VL1.M7, VH2.M8-VL1.M7, VH2.M9-VL1.M7, VH2.M10-VL1.M7, VH2.M11-VL1.M7, VH2.M12-VL1.M7, VH1-VL1.M8, VH2-VL1.M8, VH2.M1-VL1.M8, VH2.M5-VL 1.M8, VH2.M6-VL 1.M8, VH2.M8-VL 1.M8, VH2.M9-VL1.M8, VH2.M10-VL1.M8, VH2.M11-VL1.M8, VH2.M12-VL1.M8, VH1-VL1.M9, VH2-VL1.M9, VH2.M1-VL1.M9, VH2.M5-VL1.M9, VH2.M6-VL1.M9, VH2.M8-VL1.M9, VH2.M9-VL1.M9, VH2.M10-VL1.M9, VH2.M11-VL1.M9, and VH2.M12-VL1.M9. In some embodiments, the present disclosure provides antibodies and antigen-binding fragments thereof of VL1-VH2, VL1-VH2M1, VL1-VH2M5, VL1-VH2M6, VL1M2-VH2, VL1M2-VH2M1, VL1M2-VH2M5, VL1M2-VH2M6, VL1M2-VH2M8, VL1M2-VH2M9, VL1M2-VH2M10, VL1M2-VH2M11, VL1M3-VH2M6, VL1M6-VH2M6, VL1M7-VH2M6, and VL1M8-VH2M6.

In some embodiments, the VH CDR1, VH CDR2, and VH CDR3 are selected from the combination of VH CDR sequences as shown in FIG. 12B. In some embodiments, the VL CDR1, VL CDR2, and VL CDR3 are selected from the combination of VL CDR sequences as shown in FIG. 12C. Any of these VH sequences can be combined with any of these VL sequences.

In some embodiments, provided is a VH comprising VH CDR1, VH CDR2, VH CDR3, wherein (a) the VH CDR1 amino acid sequence is selected from the group consisting of SEQ ID NOs: 20 and 21; (b) the VH CDR2 amino acid sequence is selected from the group consisting of SEQ ID NOs: 22-28; (c) the VH CDR3 amino acid sequence is SEQ ID NOs: 29. In some embodiments, provided is a VL comprising VL CDR1, VL CDR2, VL CDR3, wherein (d) the VL CDR1 amino acid sequence is selected from the group consisting of SEQ ID NOs: 30 and 31; (e) the VL CDR2 amino acid sequence is selected from the group consisting of SEQ ID NOs: 32 and 33; and (f) the VL CDR3 amino acid sequence is selected from the group consisting of SEQ ID NOs: 34-37.

The CDR sequences for VL1-VH2 include CDRs of the heavy chain variable domain, SEQ ID NOs: 20, 22, and 29, and CDRs of the light chain variable domain, SEQ ID NOs: 30, 32, and 34.

The CDR sequences for VL1-VH2M1 include CDRs of the heavy chain variable domain, SEQ ID NOs: 21, 22, and 29, and CDRs of the light chain variable domain, SEQ ID NOs: 30, 32, and 34.

The CDR sequences for VL1-VH2M5 include CDRs of the heavy chain variable domain, SEQ ID NOs: 20, 23 and 29, and CDRs of the light chain variable domain, SEQ ID NOs: 30, 32, and 34.

The CDR sequences for VL1-VH2M6 include CDRs of the heavy chain variable domain, SEQ ID NOs: 21, 23, and 29, and CDRs of the light chain variable domain, SEQ ID NOs: 30, 32, and 34.

The CDR sequences for VL1M2-VH2 include CDRs of the heavy chain variable domain, SEQ ID NOs: 20, 22 and 29, and CDRs of the light chain variable domain, SEQ ID NOs: 30, 33 and 34.

The CDR sequences for VL1M2-VH2M1 include CDRs of the heavy chain variable domain, SEQ ID NOs: 21, 22, and 29, and CDRs of the light chain variable domain, SEQ ID NOs: 30, 33 and 34.

The CDR sequences for VL1M2-VH2M5 include CDRs of the heavy chain variable domain, SEQ ID NOs: 20, 23, and 29, and CDRs of the light chain variable domain, SEQ ID NOs: 30, 33 and 34.

The CDR sequences for VL1M2-VH2M6 include CDRs of the heavy chain variable domain, SEQ ID NOs: 21, 23, and 29, and CDRs of the light chain variable domain, SEQ ID NOs: 30, 33 and 34.

The CDR sequences for VL1M2-VH2M8 include CDRs of the heavy chain variable domain, SEQ ID NOs: 21, 24, and 29, and CDRs of the light chain variable domain, SEQ ID NOs: 30, 33 and 34.

The CDR sequences for VL1M2-VH2M9 include CDRs of the heavy chain variable domain, SEQ ID NOs: 21, 25, and 29, and CDRs of the light chain variable domain, SEQ ID NOs: 30, 33 and 34.

The CDR sequences for VL1M2-VH2M10 include CDRs of the heavy chain variable domain, SEQ ID NOs: 21, 26, and 29, and CDRs of the light chain variable domain, SEQ ID NOs: 30, 33 and 34.

The CDR sequences for VL1M2-VH2M1linclude CDRs of the heavy chain variable domain, SEQ ID NOs: 21, 27, and 29, and CDRs of the light chain variable domain, SEQ ID NOs: 30, 33 and 34.

The CDR sequences for VL1M3-VH2M6 include CDRs of the heavy chain variable domain, SEQ ID NOs: 21, 23 and 29, and CDRs of the light chain variable domain, SEQ ID NOs: 31, 33 and 34.

The CDR sequences for VL1M6-VH2M6 include CDRs of the heavy chain variable domain, SEQ ID NOs: 21, 23 and 29, and CDRs of the light chain variable domain, SEQ ID NOs: 30, 33 and 35.

The CDR sequences for VL1M7-VH2M6 include CDRs of the heavy chain variable domain, SEQ ID NOs: 21, 23 and 29, and CDRs of the light chain variable domain, SEQ ID NOs: 30, 33 and 36.

The CDR sequences for VL1M8-VH2M6 include CDRs of the heavy chain variable domain, SEQ ID NOs: 21, 23 and 29, and CDRs of the light chain variable domain, SEQ ID NOs: 30, 33 and 37.

Furthermore, in some embodiments, the antibodies or antigen-binding fragments thereof described herein can also contain one, two, or three heavy chain variable region CDRs selected from VH CDRs in Table 6, and one, two, or three light chain variable region CDRs selected from VL CDRs in Table 7. These CDR sequences can be determined by a combination of Kabat and AbM definitions. In some embodiments, the VL CDR1, VL CDR2, VL CDR3, VH CDR2, and VH CDR3 are determined by Kabat definitions. In some embodiments, the VH CDR1 is determined by AbM, e.g., the VH CDR1 can start 5 residues before the typical Kabat definition.

In some embodiments, the antibodies can have a heavy chain variable region (VH) comprising complementarity determining regions (CDRs) 1, 2, 3, wherein the CDR1 region comprises or consists of an amino acid sequence that is at least 80%, 85%, 90%, 95%, 96%, 97%, 98%, 99% or 100% identical to a selected VH CDR1 amino acid sequence, the CDR2 region comprises or consists of an amino acid sequence that is at least 80%, 85%, 90%, 95%, 96%, 97%, 98%, 99% or 100% identical to a selected VH CDR2 amino acid sequence, and the CDR3 region comprises or consists of an amino acid sequence that is at least 80%, 85%, 90%, 95%, 96%, 97%, 98%, 99% or 100% identical to a selected VH CDR3 amino acid sequence. In some embodiments, the antibodies can have a light chain variable region (VL) comprising CDRs 1, 2, 3, wherein the CDR1 region comprises or consists of an amino acid sequence that is at least 80%, 85%, 90%, 95%, 96%, 97%, 98%, 99% or 100% identical to a selected VL CDR1 amino acid sequence, the CDR2 region comprises or consists of an amino acid sequence that is at least 80%, 85%, 90%, 95%, 96%, 97%, 98%, 99% or 100% identical to a selected VL CDR2 amino acid sequence, and the CDR3 region comprises or consists of an amino acid sequence that is at least 80%, 85%, 90%, 95%, 96%, 97%, 98%, 99% or 100% identical to a selected VL CDR3 amino acid sequence. The selected VH CDRs 1, 2, 3 amino acid sequences and the selected VL CDRs, 1, 2, 3 amino acid sequences are shown in Table 6 and Table 7. The different combination of VH CDRs and VL CDRs are shown in FIGS. 12B and 12C.

In some embodiments, the antibody or an antigen-binding fragment described herein can contain a heavy chain variable domain containing one, two, or three of the CDRs in Table 6 with zero, one or two amino acid insertions, deletions, or substitutions in each of the CDRs. In some embodiments, the antibody or an antigen-binding fragment described herein can contain a light chain variable domain containing one, two, or three of the CDRs in Table 7 with zero, one or two amino acid insertions, deletions, or substitutions in each of the CDRs.

The amino acid sequences for heavy chain variable regions and light variable regions of the various antibodies are also provided. As there are different ways to humanize an antibody (e.g., a sequence can be modified with different amino acid substitutions), the heavy chain and the light chain of an antibody can have more than one version of humanized sequences.

The disclosure also provides antibodies or antigen-binding fragments thereof that bind to GPC3. The antibodies or antigen-binding fragments thereof contain a heavy chain variable region (VH) comprising or consisting of an amino acid sequence that is at least 80%, 85%, 90%, 95%, 96%, 97%, 98% or 99% identical to a selected VH sequence, and a light chain variable region (VL) comprising or consisting of an amino acid sequence that is at least 80%, 85%, 90%, 95%, 96%, 97%, 98% or 99% identical to a selected VL sequence. In some embodiments, the selected VH sequence is one of the following: the selected VH sequence is SEQ ID NO: 3; the selected VH sequence is SEQ ID NO: 5; the selected VH sequence is SEQ ID NO: 6; the selected VH sequence is SEQ ID NO: 7; the selected VH sequence is SEQ ID NO: 8; the selected VH sequence is SEQ ID NO: 11; the selected VH sequence is SEQ ID NO: 12; the selected VH sequence is SEQ ID NO: 13; the selected VH sequence is SEQ ID NO: 14; the selected VH sequence is SEQ ID NO: 15. In some embodiments, the selected VL sequence is one of the following: the selected VL sequence is SEQ ID NO: 4; the selected VL sequence is SEQ ID NO: 9; the selected VL sequence is SEQ ID NO: 16; the selected VL sequence is SEQ ID NO: 17; the selected VL sequence is SEQ ID NO: 18; the selected VL sequence is SEQ ID NO: 19; the selected VL sequence is SEQ ID NO: 137.

The different combination of VH and VL are shown in FIG. 12A. In some embodiments, the selected VH sequence and the selected VL sequences are derived from VH1-VL1, VH2-VL1, VH2.M1-VL1, VH2.M5-VL1, VH2.M6-VL1, VH2.M8-VL1, VH2.M9-VL1, VH2.M10-VL1, VH2.M11-VL1, VH2.M12-VL1, VH1-VL1.M2, VH2-VL1.M2, VH2.M1-VL1.M2, VH2.M5-VL1.M2, VH2.M6-VL1.M2, VH2.M8-VL1.M2, VH2.M9-VL1.M2, VH2.M10-VL1.M2, VH2.M11-VL1.M2, VH2.M12-VL1.M2, VH1-VL1.M3, VH2-VL1.M3, VH2.M1-VL1.M3, VH2.M5-VL1.M3, VH2.M6-VL1.M3, VH2.M8-VL1.M3, VH2.M9-VL1.M3, VH2.M10-VL1.M3, VH2.M11-VL1.M3, VH2.M12-VL1.M3, VH1-VL1.M6, VH2-VL1.M6, VH2.M1-VL1.M6, VH2.M5-VL1.M6, VH2.M6-VL1.M6, VH2.M8-VL1.M6, VH2.M9-VL1.M6, VH2.M10-VL1.M6, VH2.M11-VL1.M6, VH2.M12-VL1.M6, VH1-VL1.M7, VH2-VL1.M7, VH2.M1-VL1.M7, VH2.M5-VL1.M7, VH2.M6-VL1.M7, VH2.M8-VL1.M7, VH2.M9-VL1.M7, VH2.M10-VL1.M7, VH2.M11-VL1.M7, VH2.M12-VL1.M7, VH1-VL1.M8, VH2-VL1.M8, VH2.M1-VL1.M8, VH2.M5-VL1.M8, VH2.M6-VL1.M8, VH2.M8-VL1.M8, VH2.M9-VL1.M8, VH2.M10-VL1.M8, VH2.M11-VL1.M8, VH2.M12-VL1.M8, VH1-VL1.M9, VH2-VL1.M9, VH2.M1-VL1.M9, VH2.M5-VL1.M9, VH2.M6-VL1.M9, VH2.M8-VL1.M9, VH2.M9-VL1.M9, VH2.M10-VL1.M9, VH2.M11-VL1.M9, and VH2.M12-VL1.M9. In some embodiments, the selected VH sequence and the selected VL sequences are derived from VL1-VH2, VL1-VH2M1, VL1-VH2M5, VL1-VH2M6, VL1M2-VH2, VL1M2-VH2M1, VL1M2-VH2M5, VL1M2-VH2M6, VL1M2-VH2M8, VL1M2-VH2M9, VL1M2-VH2M10, VL1M2-VH2M11, VL1M3-VH2M6, VL1M6-VH2M6, VL1M7-VH2M6, and VL1M8-VH2M6.

The amino acid sequence for the heavy chain variable region of antibody VL1-VH2 is set forth in SEQ ID NO: 5. The amino acid sequence for the light chain variable region of VL1-VH2 antibody is set forth in SEQ ID NO: 4.

The amino acid sequence for the heavy chain variable region of antibody VL1-VH2.M1 is set forth in SEQ ID NO: 6. The amino acid sequence for the light chain variable region of VL1-VH2.M1 antibody is set forth in SEQ ID NO: 4.

The amino acid sequence for the heavy chain variable region of antibody VL1-VH2.M5 is set forth in SEQ ID NO: 7. The amino acid sequence for the light chain variable region of VL1-VH2.M5 antibody is set forth in SEQ ID NO: 4.

The amino acid sequence for the heavy chain variable region of antibody VL1-VH2.M6 is set forth in SEQ ID NO: 8. The amino acid sequence for the light chain variable region of VL1-VH2.M6 antibody is set forth in SEQ ID NO: 4.

The amino acid sequence for the heavy chain variable region of antibody VL1.M2-VH2 is set forth in SEQ ID NO: 5. The amino acid sequence for the light chain variable region of VL1.M2-VH2 antibody is set forth in SEQ ID NO: 9.

The amino acid sequence for the heavy chain variable region of antibody VL1.M2-VH2.M1 is set forth in SEQ ID NO: 6. The amino acid sequence for the light chain variable region of VL1.M2-VH2.M1 antibody is set forth in SEQ ID NO: 9.

The amino acid sequence for the heavy chain variable region of antibody VL1.M2-VH2.M5 is set forth in SEQ ID NO: 7. The amino acid sequence for the light chain variable region of VL1.M2-VH2.M5 antibody is set forth in SEQ ID NO: 9.

The amino acid sequence for the heavy chain variable region of antibody VL1.M2-VH2.M6 is set forth in SEQ ID NO: 8. The amino acid sequence for the light chain variable region of VL1.M2-VH2.M6 antibody is set forth in SEQ ID NO: 9.

The amino acid sequence for the heavy chain variable region of antibody VL1.M2-VH2.M8 is set forth in SEQ ID NO: 11. The amino acid sequence for the light chain variable region of VL1.M2-VH2.M8 antibody is set forth in SEQ ID NO: 9.

The amino acid sequence for the heavy chain variable region of antibody VL1.M2-VH2.M9 is set forth in SEQ ID NO: 12. The amino acid sequence for the light chain variable region of VL1M2-VH2M9 antibody is set forth in SEQ ID NO: 9.

The amino acid sequence for the heavy chain variable region of antibody VL1.M2-VH2.M10 is set forth in SEQ ID NO: 13. The amino acid sequence for the light chain variable region of VL1.M2-VH2.M10 antibody is set forth in SEQ ID NO: 9.

The amino acid sequence for the heavy chain variable region of antibody VL1.M2-VH2.M11 is set forth in SEQ ID NO: 14. The amino acid sequence for the light chain variable region of VL1.M2-VH2.M11 antibody is set forth in SEQ ID NO: 9.

The amino acid sequence for the heavy chain variable region of antibody VL1.M3-VH2.M6 is set forth in SEQ ID NO: 8. The amino acid sequence for the light chain variable region of VL1.M3-VH2.M6 antibody is set forth in SEQ ID NO: 16.

The amino acid sequence for the heavy chain variable region of antibody VL1.M6-VH2.M6 is set forth in SEQ ID NO: 8. The amino acid sequence for the light chain variable region of VL1.M6-VH2.M6 antibody is set forth in SEQ ID NO: 17.

The amino acid sequence for the heavy chain variable region of antibody VL1.M7-VH2.M6 is set forth in SEQ ID NO: 8. The amino acid sequence for the light chain variable region of VL1.M7-VH2.M6 antibody is set forth in SEQ ID NO: 18.

The amino acid sequence for the heavy chain variable region of antibody VL1.M8-VH2.M6 is set forth in SEQ ID NO: 8. The amino acid sequence for the light chain variable region of VL1.M8-VH2.M6 antibody is set forth in SEQ ID NO: 19.

The present disclosure also provides humanized antibodies or antigen binding fragments thereof. Humanization percentage means the percentage identity of the heavy chain or light chain variable region sequence as compared to human antibody sequences in International Immunogenetics Information System (IMGT) database. In some embodiments, humanization percentage is greater than 80%, 81%, 82%, 83%, 84%, 85%, 86%, 87%, 88%, 89%, 90%, 91%, 92%, 93%, 94%, or 95%. A detailed description regarding how to determine humanization percentage and how to determine top hits is known in the art, and is described, e.g., in Jones, Tim D., et al, MAbs. Vol. 8. No. 1. Taylor & Francis, 2016, which is incorporated herein by reference in its entirety. A high humanization percentage often has various advantages, e.g., more safe and more effective in humans, more likely to be tolerated by a human subject, and/or less likely to have side effects.

In some embodiments, the human antibody germlines with highest sequence homology to the variable domains can be used for humanization purpose (e.g., HV1-46 for VH or KV2-29 for VL). In some embodiments, the murine VH and VL CDRs can be straightly grafted to these human frameworks. In some embodiments, the humanized VH has the sequence of SEQ ID NO: 3 and the humanized VL has the sequence of SEQ ID NO: 4. In some embodiments, in order to restore the binding activity, back mutations in the framework regions can be introduced. In some embodiments, the back mutations include one or more of the following in the VH:M70L, R72A and A97T. In some embodiments, the humanized VH has the sequence of SEQ ID NO: 5. In some embodiments, the VH has one or more (e.g., 1, 2, 3) of the following: (1) amino acid at position 69 (Kabat numbering) is L; (2) amino acid at position 71 (Kabat numbering) is A; and (2) amino acid at position 93 (Kabat numbering) is T. To increase the humanization of CDRs, several amino acid residues in VH and VL can also be mutated to their human counterparts in HV1-46 and KV2-29 germlines, respectively. These humanized variable domains can be e.g., VH2.M1 with D31S mutation in HCDR1 (SEQ ID NO: 6), VH2.M5 with K65Q mutation in HCDR2 (SEQ ID NO: 7), VH2.M6 with both HCDR mutations (SEQ ID NO: 8) and VL1.M2 with N58S mutation in LCDR2 (SEQ ID NO: 9).

Furthermore, in some embodiments, the antibodies or antigen-binding fragments thereof described herein can also contain one, two, or three heavy chain variable region CDRs (in any order) selected from the groups of SEQ ID NOs for each antibody or antigen-binding fragment listed in Table 6, and/or one, two, or three light chain variable region CDRs (in any order) selected from the groups of SEQ ID NOs for each antibody or antigen-binding fragment listed in Table 7. In some embodiments, the antibody or an antigen-binding fragment described herein can contain a heavy chain variable domain containing one, two, or three of the CDRs of any one of the heavy chain CDRs of the antibodies or antigen-binding fragments thereof described herein with zero, one or two amino acid insertions, deletions, or substitutions. In some embodiments, the antibody or an antigen-binding fragment described herein can contain a light chain variable domain containing one, two, or three of the CDRs of any one of the light chain CDRs of the antibodies or antigen-binding fragments thereof described herein with zero, one or two amino acid insertions, deletions, or substitutions. The insertions, deletions, and substitutions can be within the CDR sequence, or at one or both terminal ends of the CDR sequence.

The disclosure also provides antibodies or antigen-binding fragments thereof that bind to GPC3. The antibodies or antigen-binding fragments thereof contain a heavy chain variable region (VH) comprising or consisting of an amino acid sequence that is at least 80%, 85%, 90%, or 95% identical to a selected VH sequence or the VH of a selected scFv, and a light chain variable region (VL) comprising or consisting of an amino acid sequence that is at least 80%, 85%, 90%, or 95% identical to a selected VL sequence or the VL of a selected scFv. In some embodiments, the selected scFv comprises a sequence selected from SEQ ID NO: 39-46 and 62-96. In some embodiments, the VH and VL are selected from the combination as shown in Table 2, Table 8, and FIG. 12A.

In some embodiments, the selected VH sequence is SEQ ID NO: 5, and the selected VL sequence is SEQ ID NO: 4. In some embodiments, the selected scFv is SEQ ID NO: 39.

In some embodiments, the selected VH sequence is SEQ ID NO: 6, and the selected VL sequence is SEQ ID NO: 4. In some embodiments, the selected scFv is SEQ ID NO: 40.

In some embodiments, the selected VH sequence is SEQ ID NO: 7, and the selected VL sequence is SEQ ID NO: 4. In some embodiments, the selected scFv is SEQ ID NO: 41.

In some embodiments, the selected VH sequence is SEQ ID NO: 8, and the selected VL sequence is SEQ ID NO: 4. In some embodiments, the selected scFv is SEQ ID NO: 42.

In some embodiments, the selected VH sequence is SEQ ID NO: 5, and the selected VL sequence is SEQ ID NO: 9. In some embodiments, the selected scFv is SEQ ID NO: 43.

In some embodiments, the selected VH sequence is SEQ ID NO: 6, and the selected VL sequence is SEQ ID NO: 9. In some embodiments, the selected scFv is SEQ ID NO: 44.

In some embodiments, the selected VH sequence is SEQ ID NO: 7, and the selected VL sequence is SEQ ID NO: 9. In some embodiments, the selected scFv is SEQ ID NO: 45.

In some embodiments, the selected VH sequence is SEQ ID NO: 8, and the selected VL sequence is SEQ ID NO: 9. In some embodiments, the selected scFv is SEQ ID NO: 46.

In some embodiments, the selected VH sequence is SEQ ID NO: 11, and the selected VL sequence is SEQ ID NO: 9.

In some embodiments, the selected VH sequence is SEQ ID NO: 12, and the selected VL sequence is SEQ ID NO: 9.

In some embodiments, the selected VH sequence is SEQ ID NO: 13, and the selected VL sequence is SEQ ID NO: 9.

In some embodiments, the selected VH sequence is SEQ ID NO: 14, and the selected VL sequence is SEQ ID NO: 9.

In some embodiments, the selected VH sequence is SEQ ID NO: 8, and the selected VL sequence is SEQ ID NO: 16.

In some embodiments, the selected VH sequence is SEQ ID NO: 8, and the selected VL sequence is SEQ ID NO: 17.

In some embodiments, the selected VH sequence is SEQ ID NO: 8, and the selected VL sequence is SEQ ID NO: 18.

In some embodiments, the selected VH sequence is SEQ ID NO: 8, and the selected VL sequence is SEQ ID NO: 19.

The disclosure also provides nucleic acid comprising a polynucleotide encoding a polypeptide comprising an immunoglobulin heavy chain or an immunoglobulin light chain. The immunoglobulin heavy chain or immunoglobulin light chain comprises CDRs of any one of the antibodies or antigen binding fragments thereof described herein, or have sequences of the immunoglobulin heavy chain or immunoglobulin light chain of any one of the antibodies or antigen binding fragments thereof described herein. When the polypeptides are paired with corresponding polypeptide (e.g., a corresponding heavy chain variable region or a corresponding light chain variable region), the paired polypeptides bind to GPC3.

In some embodiments, the nucleic acid described herein comprises a polynucleotide encoding a polypeptide comprising an immunoglobulin heavy chain or a fragment thereof comprising a heavy chain variable region (VH) comprising complementarity determining regions (CDRs) 1, 2, and 3, wherein
(a) the VH CDR1 comprises GYTFTX₁YEMH (SEQ ID NO: 132);
(b) the VH CDR2 comprises ALDPX₂×₃GX₄TAYSQKFX₅G (SEQ ID NO: 133);
(c) the VH CDR3 comprises FYSYTY(SEQ ID NO: 29);
wherein
X₁ is S or D;
X₂ is S or K;
X₃ is G or T;
X₄ is S or D;
X₅ is Q or K,
wherein the VH, when paired with a light chain variable region (VL) comprising the amino acid sequence set forth in SEQ ID NO: 4, 9, 16, 17, 18, 19 or 137, or any VL as described herein binds to GPC3.

In some embodiments, the nucleic acid described herein comprises a polynucleotide encoding a polypeptide comprising an immunoglobulin light chain or a fragment thereof comprising a VL comprising CDRs 1, 2, and 3, wherein
(d) the VL CDR1 comprises RSSQSLVHSNGX₆TYLH(SEQ ID NO: 134);
(e) the VL CDR2 comprises KVSX₇RFS(SEQ ID NO: 135); and
(f) the VL CDR3 comprises X₈QX₉THX₁₀PPT(SEQ ID NO: 136);
and wherein:
X₆ is N or K;
X₇ is N or S;
X₈ is S or M;
X₉ is N or G; and
X₁₀ is W or V,
wherein the VL, when paired with a VH comprising the amino acid sequence set forth in SEQ ID NO: 3, 5, 6, 7, 8, 11, 12, 13, 14, or 15 or any VH as described herein binds to GPC3.

In some embodiments, the nucleic acid described herein comprises a polynucleotide encoding a polypeptide comprising an immunoglobulin heavy chain or a fragment thereof comprising a VH comprising VH CDR1, VH CDR2, VH CDR3, wherein
(a) the VH CDR1 amino acid sequence is selected from the group consisting of SEQ ID NOs: 20 and 21;
(b) the VH CDR2 amino acid sequence is selected from the group consisting of SEQ ID NOs: 22-28;
(c) the VH CDR3 amino acid sequence is SEQ ID NOs: 29,
wherein the VH, when paired with a light chain variable region (VL) comprising the amino acid sequence set forth in SEQ ID NO: 4, 9, 16, 17, 18, 19 or 137 or any VL as described herein binds to GPC3.

In some embodiments, the nucleic acid described herein comprises a polynucleotide encoding a polypeptide comprising an immunoglobulin light chain or a fragment thereof comprising a VL comprising VL CDR1, VL CDR2, VL CDR3, wherein
(d) the VL CDR1 amino acid sequence is selected from the group consisting of SEQ ID NOs: 30 and 31;
(e) the VL CDR2 amino acid sequence is selected from the group consisting of SEQ ID NOs: 32 and 33; and
(f) the VL CDR3 amino acid sequence is selected from the group consisting of SEQ ID NOs: 34-37,
wherein the VL, when paired with a VH comprising the amino acid sequence set forth in SEQ ID NO: 3, 5, 6, 7, 8, 11, 12, 13, 14 or 15 or any VH as described herein binds to GPC3.

### Chimeric Antigen Receptors and Binding Molecules

Chimeric antigen receptors (CARs) combine many facets of normal T cell activation into a single protein. They link an extracellular antigen recognition domain to an intracellular signaling domain, which activates the T cell when an antigen is bound. CARs typically have the following regions: an antigen binding domain, an extracellular hinge domain, a transmembrane domain, and an intracellular domain. In some embodiments, the intracellular domain comprises an intracellular signaling domain or an intracellular signaling region.

The antigen binding domain is exposed to the outside of the cell, in the ectodomain portion of the receptor. It interacts with potential target molecules and is responsible for targeting the CAR-T cell to any cell expressing a matching molecule. The antigen binding domain is typically derived from the variable regions of a monoclonal antibody linked together as a single-chain variable fragment (scFv). An scFv is a chimeric protein made up of the light (VL) and heavy (VH) chains of immunoglobulins, connected with a short linker peptide. In some embodiments, the antigen binding domain comprises one or more (e.g., 1, 2, 3, 4, 5, or 6) VH-VL pairs. In some embodiments, the variable domains are connected with a linker peptide (e.g., a flexible linker). The linker peptide can include hydrophilic residues with stretches of glycine and serine in it for flexibility as well as stretches of glutamate and lysine for added solubility.

In some embodiments, the linker peptide comprises at least or about 5, 6, 7, 8, 9, 10, 11, 12, 13, 14, 15, 16, 17, 18, 19, 20, 30, 40, or 50 amino acid residues. In some embodiments, the linker peptide comprises at least or about 4, 5, 6, 7, 8, 9, 10, 11, 12, 13, 14, 15, 16, 20, 25, 30, or 40 glycine residues. In some embodiments, the linker peptide comprises at least or about 1, 2, 3, 4, 5, 6, 7, or 8 serine residues. In some embodiments, the linker peptide comprises or consists of both glycine and serine residues. In some embodiments, the linker peptide comprises or consists of a sequence that is at least or about 70%, at least or about 75%, at least or about 80%, at least or about 85%, at least or about 90%, at least or about 95%, at least or about 99%, or 100% identical to GGGGS (SEQ ID NO: 140) or GGGGSGGGGSGGGGS (SEQ ID NO: 10). In some embodiments, the linker sequence comprises at least 1, 2, 3, 4, 5, 6, 7, or 8 repeats of GGGGS (SEQ ID NO: 140). In some embodiments, the linker sequence has no more than 10, 11, 12, 13, 14, 15, 16, 17, 18, 19, 20, 30, 40, or 50 amino acid residues. In some embodiments, the linker peptide comprises 1, 2, 3, 4, or 5 amino acid insertions, deletions, or substitutions.

In some embodiments, the antigen binding domain specifically binds to GPC3 (e.g., human GPC3, mouse GPC3, or monkey (cynomolgus) GPC3). In some embodiments, the antigen binding domain specifically binds to the extracellular domain (ECD) of GPC3.

The hinge, also called a spacer, is a small structural domain that sits between the antigen binding domain and the cell's outer membrane. An ideal hinge enhances the flexibility of the antigen binding domain, reducing the spatial constraints between the CAR and its target antigen. This promotes antigen binding and synapse formation between the CAR-T cells and target cells. Hinge sequences are often based on IgG hinge regions, or membrane-proximal regions from immune molecules including e.g., CD8, and CD28. In some embodiments, the hinge region is derived from CD8.

The transmembrane region or transmembrane domain is a structural component, consisting of a hydrophobic alpha helix that spans the cell membrane. It anchors the CAR to the plasma membrane, bridging the extracellular hinge and antigen binding domains with the intracellular signaling domain. This domain is essential for the stability of the receptor as a whole. Generally, the transmembrane domain from the most membrane-proximal component of the endodomain is used, but different transmembrane domains result in different receptor stability. In some embodiments, the transmembrane region is derived from CD8.

The intracellular T cell signaling region lies in the receptor's endodomain, inside the cell. After an antigen is bound to the external antigen binding domain, CAR receptors cluster together and transmit an activation signal. Then the internal cytoplasmic end of the receptor perpetuates signaling inside the T cell. Normal T cell activation relies on the phosphorylation of immunoreceptor tyrosine-based activation motifs (ITAMs) present in the cytoplasmic domain of CD3-zeta. To mimic this process, CD3-zeta's cytoplasmic domain is commonly used as the main CAR endodomain component. T cells also require co-stimulatory molecules in addition to CD3 signaling in order to persist after activation. For this reason, the endodomains of CAR receptors typically also include one or more chimeric domains from co-stimulatory proteins. Signaling domains from a wide variety of co-stimulatory molecules have been successfully tested, including CD28, CD27, CD134 (OX40), ICOS, hematopoietic cell signal transducer (DAP10) and/or CD137 (4-11B). In some embodiments, the co-stimulatory domain is derived from 4-1BB.

Various CAR molecules and vectors expressing these CAR molecules can be used in the methods described herein. In some embodiments, the CAR molecules specifically binds to GPC3 (e.g., human GPC3). In some embodiments, the CAR comprises the amino acid sequence set forth in any of SEQ ID NOs: 54-61 and 97-131; or an amino acid sequence that has at least 80%, 85%, 90%, 91%, 92%, 93%, 94%, 95%, 96%, 97%, 98%, or 99% sequence identity thereto.

Exemplary structure of antigen receptors, including the hinge, the transmembrane domain, and the intracellular T cell signaling domain, and methods for engineering and introducing such receptors into cells, are described, for example, in Chandran et al., Immunological Reviews 290.1 (2019): 127-147; Cartellieri, Marc, et al., BioMed Research International 2010 (2010); and PCT Publication No. WO2017173256A1; US2002/131960, US2013/287748, US2013/0149337, U.S. Pat. Nos. 6,451,995, 7,446,190, and 8,252,592; each of which is incorporated herein by reference in its entirety.

The disclosure provides chimeric antigen receptors (CARs) or fragments thereof that specifically bind to GPC3. The CARs or fragments thereof described herein are capable of binding to GPC3.

The disclosure provides CARs or fragments thereof, comprising (a) an extracellular antigen-binding domain that specifically recognizes GPC3; (b) a transmembrane domain or region; and/or (c) an intracellular signaling domain. In some embodiments, the antigen-binding domain of the CARs or fragments thereof described herein are identical to any of the antigen binding fragments described herein (e.g., VH1-VL1, VH2-VL1, VH2.M1-VL1, VH2.M5-VL1, VH2.M6-VL1, VH2.M8-VL1, VH2.M9-VL1, VH2.M10-VL1, VH2.M11-VL1, VH2.M12-VL1, VH1-VL1.M2, VH2-VL1.M2, VH2.M1-VL1.M2, VH2.M5-VL1.M2, VH2.M6-VL1.M2, VH2.M8-VL1.M2, VH2.M9-VL1.M2, VH2.M10-VL1.M2, VH2.M11-VL1.M2, VH2.M12-VL1.M2, VH1-VL1.M3, VH2-VL1.M3, VH2.M1-VL1.M3, VH2.M5-VL1.M3, VH2.M6-VL1.M3, VH2.M8-VL1.M3, VH2.M9-VL1.M3, VH2.M10-VL1.M3, VH2.M11-VL1.M3, VH2.M12-VL1.M3, VH1-VL1.M6, VH2-VL1.M6, VH2.M1-VL1.M6, VH2.M5-VL1.M6, VH2.M6-VL1.M6, VH2.M8-VL1.M6, VH2.M9-VL1.M6, VH2.M10-VL1.M6, VH2.M11-VL1.M6, VH2.M12-VL1.M6, VH1-VL1.M7, VH2-VL1.M7, VH2.M1-VL1.M7, VH2.M5-VL1.M7, VH2.M6-VL1.M7, VH2.M8-VL1.M7, VH2.M9-VL1.M7, VH2.M10-VL1.M7, VH2.M11-VL1.M7, VH2.M12-VL1.M7, VH1-VL1.M8, VH2-VL1.M8, VH2.M1-VL1.M8, VH2.M5-VL1.M8, VH2.M6-VL 1.M8, VH2.M8-VL 1.M8, VH2.M9-VL 1.M8, VH2.M10-VL1.M8, VH2.M11-VL1.M8, VH2.M12-VL1.M8, VH1-VL1.M9, VH2-VL1.M9, VH2.M1-VL1.M9, VH2.M5-VL1.M9, VH2.M6-VL1.M9, VH2.M8-VL1.M9, VH2.M9-VL1.M9, VH2.M10-VL1.M9, VH2.M11-VL1.M9, and VH2.M12-VL1.M9 as shown in FIG. 12A). In some embodiments, the antigen-binding domain of the CARs or fragments thereof described herein comprises one or more (e.g., 1, 2, 3, 4, 5, or 6) scFvs that are connected with the linker peptide described herein.

In some embodiments, the amino acid sequences for scFv of the antigen-binding domain for the CAR, or related antigen binding fragment thereof are humanized (e.g., a sequence can be modified with different amino acid substitutions). In some embodiments, the scFv can have more than one version of humanized sequences.

In some embodiments, the CAR, related antibody or antigen binding fragment thereof described herein can have a heavy chain variable domain (VH) comprising complementarity determining regions (CDRs) 1, 2, 3, wherein the CDR1 region comprises or consists of an amino acid sequence that is at least 80%, 85%, 90%, or 95% identical to a selected VH CDR1 amino acid sequence, the CDR2 region comprises or consists of an amino acid sequence that is at least 80%, 85%, 90%, or 95% identical to a selected VH CDR2 amino acid sequence, and the CDR3 region comprises or consists of an amino acid sequence that is at least 80%, 85%, 90%, or 95% identical to a selected VH CDR3 amino acid sequence.

In some embodiments, the CAR, related antibody or antigen binding fragment thereof described herein can have a light chain variable domain (VL) comprising complementarity determining regions (CDRs) 1, 2, 3, wherein the CDR1 region comprises or consists of an amino acid sequence that is at least 80%, 85%, 90%, or 95% identical to a selected VL CDR1 amino acid sequence, the CDR2 region comprises or consists of an amino acid sequence that is at least 80%, 85%, 90%, or 95% identical to a selected VL CDR2 amino acid sequence, and the CDR3 region comprises or consists of an amino acid sequence that is at least 80%, 85%, 90%, or 95% identical to a selected VL CDR3 amino acid sequence.

In some embodiments, the selected VH and VL CDRs 1, 2, 3 amino acid sequences are shown in Tables 6-7.

In some embodiments, (a) the VH CDR1 amino acid sequence is selected from the group consisting of SEQ ID NOs: 20 and 21; (b) the VH CDR2 amino acid sequence is selected from the group consisting of SEQ ID NOs: 22-28; (c) the VH CDR3 amino acid sequence is SEQ ID NOs: 29; (d) the VL CDR1 amino acid sequence is selected from the group consisting of SEQ ID NOs: 30 and 31; (e) the VL CDR2 amino acid sequence is selected from the group consisting of SEQ ID NOs: 32 and 33; and (f) the VL CDR3 amino acid sequence is selected from the group consisting of SEQ ID NOs: 34-37.

In some embodiments, the VH CDR1, VH CDR2, and VH CDR3 are selected from the combination of VH CDR sequences as shown in FIG. 12B. In some embodiments, the VL CDR1, VL CDR2, and VL CDR3 are selected from the combination of VL CDR sequences as shown in FIG. 12C. Any of these VH sequences can be combined with any of these VL sequences.

In some embodiments, the CAR, related antibody or antigen binding fragment thereof described herein contains a VH containing one, two, or three of the VH CDR1 with zero, one or two amino acid insertions, deletions, or substitutions; VH CDR2 with zero, one or two amino acid insertions, deletions, or substitutions; VH CDR3 with zero, one or two amino acid insertions, deletions, or substitutions.

In some embodiments, the CAR, related antibody or antigen binding fragment thereof described herein contains a VL containing one, two, or three of the VL CDR1 with zero, one or two amino acid insertions, deletions, or substitutions; VL CDR2 with zero, one or two amino acid insertions, deletions, or substitutions; VL CDR3 with zero, one or two amino acid insertions, deletions, or substitutions.

The disclosure also provides CARs or fragments thereof that bind to GPC3. In some embodiments, the CAR, related antibody or antigen binding fragment thereof contains a heavy chain variable region (VH) comprising or consisting of an amino acid sequence that is at least 80%, 85%, 90%, or 95% identical to a selected VH sequence. In some embodiments, the selected VH sequence is selected from SEQ ID NOs: 3, 5-8, and 11-15.

In some embodiments, the CAR, related antibody or antigen binding fragment thereof contains a light chain variable region (VL) comprising or consisting of an amino acid sequence that is at least 80%, 85%, 90%, or 95% identical to a selected VL sequence. In some embodiments, the selected VL sequence is selected from SEQ ID NOs: 4, 9, 16-19 and 137.

Various different combinations of VH and VL are shown in FIG. 12A.

The amino acid sequence for VL1-VH2 CAR is set forth in SEQ ID NO: 39. The amino acid sequence for VL1-VH2M1CAR is set forth in SEQ ID NO: 40. The amino acid sequence for VL1-VH2M5 CAR is set forth in SEQ ID NO: 41. The amino acid sequence for VL1-VH2M6 CAR is set forth in SEQ ID NO: 42. The amino acid sequence for VL1M2-VH2 CAR is set forth in SEQ ID NO: 43. The amino acid sequence for VL1M2-VH2M1 CAR is set forth in SEQ ID NO: 44. The amino acid sequence for VL1M2-VH2M5 CAR is set forth in SEQ ID NO: 45. The amino acid sequence for VL1M2-VH2M6 CAR is set forth in SEQ ID NO: 46.

In some embodiments, provided herein are CARs or fragments thereof comprising an amino acid sequence that is at least 90%, 91%, 92%, 93%, 94%, 95%, 96%, 97%, 98%, 99% or 100% identical to any one of SEQ ID NOs: 54-61 and 97-131. In some embodiments, the CAR described herein comprises an amino acid sequence as set forth in any one of SEQ ID NOs: 54-61 and 97-131; optionally with about or no more than 0, 1, 2, 3, 4, 5, 6, 7, 8, 9, 10, 15, 20, 25, 30, 35, 40, 45, or 50 amino acid insertions, deletions, or substitutions.

In some embodiments, the chimeric antigen receptors (CARs) or fragments thereof described herein comprises a hinge region. In some embodiments, the hinge region is a membrane-proximal region from CD8, and/or CD28, or an IgG hinge region, or any combination thereof. In some embodiments, the hinge region is a membrane-proximal region of CD8 (e.g., human CD8). In some embodiments, the hinge region is a fusion peptide comprising all or a portion of the membrane-proximal region of CD28 (e.g., human CD28) and all or a portion of the membrane-proximal region of CD8 (e.g., human CD8). In some embodiments, the hinge region comprises the membrane-proximal regions of both CD8 and CD28.

In some embodiments, the chimeric antigen receptors (CARs) or fragments thereof described herein comprises a transmembrane region. In some embodiments, the transmembrane region is a transmembrane region of 4-1BB/CD137, an alpha chain of a T cell receptor, a beta chain of a T cell receptor, CD3 epsilon, CD4, CD5, CD8, CD8 alpha, CD9, CD16, CD19, CD22, CD28, CD33, CD37, CD45, CD64, CD80, CD86, CD134, CD137, CD154, or a zeta chain of a T cell receptor, or any combination thereof. In some embodiments, the transmembrane region is a transmembrane region from CD8 (e.g., human CD8). In some embodiments, the hinge region and the transmembrane region are directly joined. In some embodiments, the transmembrane region is a fusion peptide comprising all or a portion of the transmembrane region of CD28 (e.g., human CD28) and all or a portion of the transmembrane region of CD8 (e.g., human CD8). In some embodiments, the transmembrane region comprises the transmembrane regions of both CD8 and CD28.

In some embodiments, the chimeric antigen receptors (CARs) or fragments thereof described herein comprises an intracellular signaling domain. In some embodiments, the intracellular signaling domain comprises an activating cytoplasmic signaling domain, which is capable of inducing a primary activation signal in an immune cell (e.g., a T cell). In some embodiments, the activating cytoplasmic signaling domain is a T cell receptor (TCR) component. In some embodiments, the activating cytoplasmic signaling domain comprises an immunoreceptor tyrosine-based activation motif (ITAM). In some embodiments, the intracellular signaling domain comprises an amino acid sequence derived from CD3 zeta, FcR gamma, FcR beta, CD3 gamma, CD3 delta, CD3 epsilon, CD5, CD22, CD79a, CD79b, CD278 (ICOS), FceRI, CD66d, DAP10, DAP12, or combinations thereof. In some embodiments, the intracellular signaling domain comprises a functional signaling domain of CD3 zeta (e.g., a human CD3 zeta). In some embodiments, the intracellular signaling domain comprises an amino acid sequence that is at least 80%, at least 85%, at least 90%, at least 95%, or 100% identical to SEQ ID NO: 52.

In some embodiments, the chimeric antigen receptors (CARs) or fragments thereof described herein comprises a costimulatory signaling domain. In some embodiments, the costimulatory signaling domain is between the transmembrane domain and the intracellular signaling domain. In some embodiments, the costimulatory signaling domain comprises a functional signaling domain from a protein selected from the group consisting of a MHC class I molecule, a TNF receptor protein, an Immunoglobulin-like protein, a cytokine receptor, an integrin, a signaling lymphocytic activation molecule (SLAM protein), an activating NK cell receptor, BTLA, a Toll ligand receptor, OX40, CD2, CD7, CD27, CD28, CD30, CD40, CDS, ICAM-1, LFA-1, CD11a/CD18, 4-1BB (CD137), B7-H3, CDS, ICAM-1, ICOS (CD278), GITR, BAFFR, LIGHT, HVEM (LIGHTR), KIRDS2, SLAMF7, NKp80 (KLRF1), NKp44, NKp30, NKp46, CD19, CD4, CD8alpha, CD8beta, IL2R beta, IL2R gamma, IL7R alpha, ITGA4, VLA1, CD49a, ITGA4, IA4, CD49D, ITGA6, VLA-6, CD49f, ITGAD, CD11d, ITGAE, CD103, ITGAL, CD11a, LFA-1, ITGAM, CD 11b, ITGAX, CD 11c, ITGB1, CD29, ITGB2, CD18, LFA-1, ITGB7, NKG2D, NKG2C, TNFR2, TRANCE/RANKL, DNAM1 (CD226), SLAMF4 (CD244, 2B4), CD84, CD96 (Tactile), CEACAMI, CRTAM, Ly9 (CD229), CD160 (BY55), PSGL1, CD100 (SEMA4D), CD69, SLAMF6 (NTB-A, Lyl08), SLAM (SLAMF1, CD150, IPO-3), BLAME (SLAMF8), SELPLG (CD162), LTBR, LAT, GADS, SLP-76, PAG/Cbp, CD19a, and a CD83 ligand. In some embodiments, the costimulatory signaling domain comprises a functional signaling domain from OX40, CD28, 4-1BB, ICOS, or a signaling portion thereof. In some embodiments, the costimulatory signaling domain comprises an intracellular signaling domain of 4-1BB (e.g., human 4-1BB). In some embodiments, the costimulatory signaling domain comprises an amino acid sequence that is at least 80%, at least 85%, at least 90%, at least 95%, or 100% identical to SEQ ID NO: 51. In some embodiments, the costimulatory signaling domain comprises an intracellular signaling domain of CD28 (e.g., human CD28). In some embodiments, the costimulatory signaling domain comprises intracellular signaling domains of both CD28 (e.g., human CD28) and 4-1BB (e.g., human 4-1BB). In some embodiments, the costimulatory signaling domain is a fusion peptide comprising all or a portion of the intracellular signaling domain of CD28 (e.g., human CD28) and all or a portion of the intracellular signaling domain of 4-1BB. In some embodiments, the costimulatory signaling domain is human CD28-4-1BB.

In some embodiments, the hinge domain, transmembrane domain, and/or intracellular signaling domain (e.g., costimulatory signaling domain and/or activating cytoplasmic signaling domain) of CARs or fragments thereof described herein are derived from a first generation, a second generation, a third generation, or a fourth generation CAR structure. Details of the structural features of CARs can be found, e.g., in Jackson, Hollie J., et al., Nature Reviews Clinical Oncology 13.6 (2016): 370; and Subklewe, Marion, et al., Transfusion Medicine and Hemotherapy 46.1 (2019): 15-24; each of which is incorporated herein by reference.

In some embodiments, the CAR is dual chain CAR, ligand-based CAR, T cell receptor fusion constructs (TRuCs), universal immune receptors (UIR), or tandem CARs (tanCARs). In some embodiments, the CAR is used in connection with bispecific T cell engagers (BiTEs). These CAR constructs are described e.g., Hughes-Parry et al, International journal of molecular sciences 21.1 (2020): 204, which is incorporated herein by reference in its entirety.

### Car, Antibody, Antigen-Binding Fragment Characteristics

In some embodiments, the CAR, antibodies, or antigen-binding fragments thereof as described herein can increase immune response, activity or number of immune cells (e.g., T cells, CD8+ T cells, CD4+ T cells, macrophages, antigen presenting cells) by at least 10%, 20%, 30%, 40%, 50%, 60%, 70%, 80%, 90%, 100%, 2 folds, 3 folds, 5 folds, 10 folds, or 20 folds, as compared to that of immune cells that do not express the CAR, antibodies, or antigen-binding fragments thereof.

In some implementations, the antibody (or antigen-binding fragments thereof) specifically binds to GPC3 with a dissociation rate (k_{off} or K_{d}) of less than 0.1 s⁻¹, less than 0.01 s⁻¹, less than 0.001 s⁻¹, less than 0.0001 s⁻¹, or less than 0.00001 s⁻¹. In some embodiments, the dissociation rate (k_{off}) is greater than 0.01 s⁻¹, greater than 0.001 s⁻¹, greater than 0.0001 s⁻¹, greater than 0.00001 s¹, or greater than 0.000001 s⁻¹.

In some embodiments, kinetic association rates (kₒₙ or K_{d}) is greater than 1×10²/Ms, greater than 1×10³/Ms, greater than 1×10⁴/Ms, greater than 1×10⁵/Ms, or greater than 1×10⁶/Ms.

In some embodiments, kinetic association rates (kₒₙ) is less than 1×10⁵/Ms, less than 1×10⁶/Ms, or less than 1×10⁷/Ms.

Binding affinities can be deduced from the quotient of the kinetic rate constants (K_{D}=k_{off}/kₒₙ). In some embodiments, K_{D} (K_{d}) for the antibody, antigen-binding fragments thereof, or molecules derived therefrom (e.g., CAR), is less than 1×10⁻⁶ M, less than 1×10⁻⁷ M, less than 1×10⁻⁸ M, less than 1×10⁻⁹ M, or less than 1×10⁻¹⁰ M. In some embodiments, the K_{D} is less than 100 nM, 50 nM, 30 nM, 20 nM, 15 nM, 10 nM, 9 nM, 8 nM, 7 nM, 6 nM, 5 nM, 4 nM, 3 nM, 2 nM, 1 nM, 0.9 nM, 0.8 nM, 0.7 nM, 0.6 nM, 0.5 nM, 0.4 nM, 0.3 nM, 0.2 nM, or 0.1 nM. In some embodiments, K_{D} is greater than 1×10⁻⁷ M, greater than 1×10⁻⁸ M, greater than 1×10⁻⁹ M, greater than 1×10⁻¹⁰ M, greater than 1×10⁻¹¹ M, or greater than 1×10⁻¹² M.

General techniques for measuring the affinity of an antibody for an antigen include, e.g., enzyme-linked immunosorbent assay (ELISA), Radioimmunoassay (RIA), fluorescence-activated cell sorting (FACS), and surface plasmon resonance (SPR). In some embodiments, the antibody binds to human GPC3. In some embodiments, the antibody binds to the extracellular domain (ECD) of human GPC3. In some embodiments, the antibody binds to monkey GPC3 (e.g., cynomolgus). In some embodiments, the antibody binds to a cell expressing GPC3.

### Engineered Cells

The present disclosure provides engineered cells (e.g., immune cells, T cells, NK cells, tumor-infiltrating lymphocytes) that express CAR, and/or various proteins as described herein. These engineered cells can be used to treat various disorders or disease as described herein (e.g., GPC3-associated cancer).

In various embodiments, the cell that is engineered can be obtained from e.g., humans and non-human animals. In various embodiments, the cell that is engineered can be obtained from bacteria, fungi, humans, rats, mice, rabbits, monkeys, pig or any other species. Preferably, the cell is from humans, rats or mice. In some embodiments, the cells are mouse lymphocytes and engineered (e.g., transduced) to express the CAR, or antigen-binding fragment thereof. In some embodiments, the cell is obtained from humans. In various embodiments, the cell that is engineered is a blood cell. Preferably, the cell is a leukocyte (e.g., a T cell), lymphocyte or any other suitable blood cell type. In some embodiments, the cell is a peripheral blood cell. In some embodiments, the cell is a tumor-infiltrating lymphocyte (TIL). In some embodiments, the cell is a T cell, B cell or NK cell. In some embodiments, the cells are human peripheral blood mononuclear cells (PBMCs). In some embodiments, the human PBMCs are CD3+ cells. In some embodiments, the human PBMCs are CD8+ cells or CD4+ cells.

In some embodiments, the cell is a T cell. In some embodiments, the T cells can express a cell surface receptor that recognizes a specific antigenic moiety on the surface of a target cell. The cell surface receptor can be a wild type or recombinant T cell receptor (TCR), a chimeric antigen receptor (CAR), or any other surface receptor capable of recognizing an antigenic moiety that is associated with the target cell. T cells can be obtained by various methods known in the art, e.g., in vitro culture of T cells (e.g., tumor infiltrating lymphocytes) isolated from patients. Genetically modified T cells can be obtained by transducing T cells (e.g., isolated from the peripheral blood of patients), with a viral vector. In some embodiments, the T cells are CD4+ T cells, CD8+ T cells, or regulatory T cells. In some embodiments, the T cells are T helper type 1 T cells and T helper type 2 T cells. In some embodiments, the T cell expressing this receptor is an αβ-T cell. In alternate embodiments, the T cell expressing this receptor is a γδ-T cell. In some embodiments, the T cells are central memory T cells. In some embodiments, the T cells are effector memory T cells. In some embodiments, the T cells are naïve T cells.

In some embodiments, the cell is an NK cell. In some embodiments, preparation of the engineered cells includes one or more culture and/or preparation steps. The cells for introduction of the binding molecule, e.g., CAR, can be isolated from a sample, such as a biological sample, e.g., one obtained from or derived from a subject. In some embodiments, the subject from which the cell is isolated is one having the disease or condition or in need of a cell therapy or to which cell therapy will be administered. The subject in some embodiments is a human in need of a particular therapeutic intervention, such as the adoptive cell therapy for which cells are being isolated, processed, and/or engineered.

In some embodiments, the cells are stem cells, such as multipotent and pluripotent stem cells, including induced pluripotent stem cells (iPSCs). The cells can be primary cells, such as those isolated directly from a subject and/or isolated from a subject and frozen. In some embodiments, the stem cells are cultured with additional differentiation factors to obtain desired cell types (e.g., T cells).

Different cell types can be obtained from appropriate isolation methods. The isolation methods include the separation of different cell types based on the expression or presence in the cell of one or more specific molecules, such as surface markers, e.g., surface proteins, intracellular markers, or nucleic acid. In some embodiments, any known method for separation based on such markers can be used. In some embodiments, the separation is affinity- or immunoaffinity-based separation. For example, the isolation in some aspects includes separation of cells and cell populations based on the cells' expression or expression level of one or more markers, typically cell surface markers, for example, by incubation with an antibody or binding partner that specifically binds to such markers, followed generally by washing steps and separation of cells having bound the antibody or binding partner, from those cells having not bound to the antibody or binding partner.

Such separation steps can be based on positive selection, in which the cells having bound the reagents are retained for further use, and/or negative selection, in which the cells having not bound to the antibody or binding partner are retained. In some examples, both fractions are retained for further use. In some aspects, negative selection can be particularly useful where no antibody is available that specifically identifies a cell type in a heterogeneous population, such that separation is best carried out based on markers expressed by cells other than the desired population.

Also provided are methods, nucleic acids, compositions, and kits, for expressing the binding molecules, and for producing the genetically engineered cells expressing such binding molecules. The genetic engineering generally involves introduction of a nucleic acid encoding the therapeutic molecule, e.g. CAR, polypeptides, fusion proteins, into the cell, such as by retroviral transduction, transfection, or transformation. In some embodiments, gene transfer is accomplished by first stimulating the cell, such as by combining it with a stimulus that induces a response such as proliferation, survival, and/or activation, e.g., as measured by expression of a cytokine or activation marker, followed by transduction of the activated cells, and expansion in culture to numbers sufficient for clinical application.

In some embodiments, recombinant nucleic acids are transferred into cells using recombinant infectious virus particles, such as, e.g., vectors derived from simian virus 40 (SV40), adenoviruses, adeno-associated virus (AAV). In some embodiments, recombinant nucleic acids are transferred into T cells using recombinant lentiviral vectors or retroviral vectors, such as gamma-retroviral vectors. In some embodiments, the retroviral vector has a long terminal repeat sequence (LTR), e.g., a retroviral vector derived from the Moloney murine leukemia virus (MoMVLV), myeloproliferative sarcoma virus (MPSV), murine embryonic stem cell virus (MESV), murine stem cell virus (MSCV), or spleen focus forming virus (SFFV). Most retroviral vectors are derived from murine retroviruses. In some embodiments, the retroviruses include those derived from any avian or mammalian cell source. The retroviruses typically are amphotropic, meaning that they are capable of infecting host cells of several species, including humans. In some embodiments, the vector is a lentivirus vector. In some embodiments, recombinant nucleic acids are transferred into T cells via electroporation. In some embodiments, recombinant nucleic acids are transferred into T cells via transposition. Other methods of introducing and expressing genetic material in immune cells include calcium phosphate transfection, protoplast fusion, cationic liposome-mediated transfection; tungsten particle-facilitated microparticle bombardment and strontium phosphate DNA co-precipitation. Many of these methods are descried e.g., in WO2019195486, which is incorporated herein by reference in its entirety. In some embodiments, the T cells are pre-activated, e.g., using anti-CD3/CD28 particles, for about 12 hours, about 24 hours, about 36 hours, about 48 hours, or about 60 hours prior to transduction. In some embodiments, the transduced T cells are harvested on day 5, day 6, day 7, day 8, day 9, day 10, day 11, or day 12 post transduction.

In some embodiments, the transfection efficiency of the virus-infected T cells described herein is at least 20%, at least 25%, at least 30%, at least 35%, at least 40%, at least 45%, at least 50%, at least 55%, at least 60%, at least 65%, at least 70%, at least 75%, or at least 80%. In some embodiments, the viability of the transduced T cells is at least 85%, at least 86%, at least 87%, at least 88%, at least 89%, at least 90%, at least 91%, at least 92%, at least 93%, at least 94%, or at least 95% on day 0, day 1, day 2, day 3, day 4, or day 5 post transduction. In some embodiments, the viability of the transduced T cells is at least or about 80%, at least or about 90%, at least or about 100%, at least or about 110%, at least or about 120% as compared to the viability of untransduced T cells, on day 0, day 1, day 2, day 3, day 4, or day 5 (e.g., on day 5) post transduction.

In some embodiments, the T cell expansion fold is at least 1 fold, 2 folds, 3 folds, 4 folds, 5 folds, 10 folds, 15 folds, 20 folds, 25 folds, 30 folds, 35 folds, 40 folds, 45 folds, or 50 folds, on day 0, day 1, day 2, day 3, day 4, or day 5 post transduction. In some embodiments, the T cell expansion fold of the transduced T cells is at least or about 50%, at least or about 60%, at least or about 70%, at least or about 80%, at least or about 90% as compared to that of untransduced T cells, on day 0, day 1, day 2, day 3, day 4, or day 5 (e.g., on day 5) post transduction.

Also provided are populations of engineered cells, compositions containing such cells and/or enriched for such cells, such as in which cells expressing the CAR make up at least 15%, 20%, 25%, 30%, 35%, 40%, 50%, 60%, 70%, 80%, 90%, 91%, 92%, 93%, 94%, 95%, 96%, 97%, 98%, 99%, or more percent of the total cells in the composition or cells of a certain type such as T cells, CD8+ or CD4+ cells.

In some embodiments, the engineered cells (e.g., CAR-T cells) are co-cultured with target cells for at least or about 1 hour, 2 hours, 3 hours, 4 hours, 5 hours, 6 hours, 12 hours, 16 hours, 18 hours, 1 day, 2 days, 3 days, or longer, such that the engineered cells (e.g., CAR-T cells) can be activated.

In some embodiments, the in vitro cytotoxicity of the engineered cells described herein (e.g., CAR-T cells) is determined. In some embodiments, the engineered cells are incubated with the target cells at an E:T ratio of about 5:1, about 4:1, about 3:1, about 2:1, about 1:1, about 0.9:1, about 0.8:1, about 0.7:1, about 0.6:1, about 0.5:1, about 0.4:1, about 0.3:1, about 0.2:1, or about 0.1:1. In some embodiments, the incubation is about 8 hours, about 10 hours, about 12 hours, about 14 hours, about 16 hours, about 20 hours, about 22 hours, about 24 hours, about 36 hours, or about 48 hours.

In some embodiment, the in vitro cytotoxicity of the engineered cells described herein (e.g., CAR-T cells) is at least 15%, at least 20%, at least 30%, at least 40%, at least 50%, at least 60%, at least 70%, at least 80%, at least 90%, or at least 95%, e.g., using the method as described in the Examples.

In some embodiments, the long-term cytotoxicity of the engineered cells (e.g., CAR-T cells) is determined, e.g., by re-challenging the engineered cells. Exemplary re-challenging procedures of CAR-T cells can be found, e.g., in Wang, Dongrui, et al., Journal of Visualized Experiments: JoVE 144 (2019); Wang D, et al., JCI Insight 2018, 3(10); Lange et al., Cancer Discov. 2021 Feb. 9, candisc.0896.2020; each of which is incorporated herein by reference in its entirety.

In some embodiments, the engineered cells are re-challenged for at least 1, 2, 3, 4, 5, or 6 times. In some embodiments, the calculated cytotoxicity (Cytotoxicity %) is determined after each re-challenge. In some embodiments, after the first re-challenged, the calculated cytotoxicity of the engineered cells described herein is at least 70%, at least 75%, at least 80%, at least 85%, at least 90%, or at least 95%. In some embodiments, after the second re-challenge, the calculated cytotoxicity of the engineered cells described herein is at least 80%, at least 90%, or at least 95%. In some embodiments, after the third re-challenge, the calculated cytotoxicity of the engineered cells described herein is at least 30%, at least 40%, at least 50%, at least 60%, at least 70%, at least 75%, at least 80%, at least 85%, at least 90%, or at least 95%. In some embodiments, after the fourth re-challenge, the calculated cytotoxicity of the engineered cells described herein is at least 15%, at least 20%, at least 30%, at least 40%, at least 50%, at least 60%, at least 70%, at least 80%, at least 90%, or at least 95%. In some embodiments, after the fifth re-challenge, the calculated cytotoxicity of the engineered cells described herein is at least 15%, at least 20%, at least 30%, at least 40%, at least 50%, at least 60%, at least 70%, at least 80%, at least 90%, or at least 95%. In some embodiments, after the sixth re-challenge, the calculated cytotoxicity of the engineered cells described herein is at least 0%, at least 5%, at least 10%, at least 15%, at least 20%, at least 30%, at least 40%, at least 50%, at least 60%, at least 70%, at least 80%, at least 90%, or at least 95%. In some embodiments, the maximum re-challenge number (i.e., the number of re-challenge times before tumor cells outgrow) of the engineered cells described herein is at least 5 times, 6 times, 7 times, 8 times, 9 times, or 10 times.

In some embodiments, when the engineered cells (e.g., CAR-T cells) are co-cultured with target cells, population of the engineered cells increases by at least or about 1 fold, 2 folds, 3 folds, 4 folds, 5 folds, 10 folds, 20 folds, 30 folds, 40 folds, 50 folds, 60 folds, 70 folds, 80 folds, 90 folds, 100 folds, 150 folds, 200 folds, or more, after 1 re-challenge, 2 re-challenges, 3 re-challenges, 4 re-challenges, 5 re-challenges, or 6 re-challenges, as compared to the initial population of the engineered cells.

In some embodiments, concentration of the cytokines (e.g., IFN-7, GM-CSF, and/or TNF-α) released by the engineered cells (e.g., CAR-T cells) described herein is determined by homogeneous time resolved fluorescence (HTRF) assays.

In some embodiments, the engineered cells (e.g. CAR-T cells) described herein increase cytokine (e.g., IFN-γ, GM-CSF, and/or TNF-α) expression or secretion by at least or about 1 fold, 2 folds, 3 folds, 4 folds, 5 folds, 10 folds, 20 folds, 30 folds, 40 folds, 50 folds, 60 folds, 70 folds, 80 folds, 90 folds, 100 folds, 500 folds, 1000 folds, 2000 folds, 3000 folds, 4000 folds, 5000 folds, 10000 folds, or more when co-cultured with the target cells, as compared to the cytokine expression or secretion level of the untransduced cells (e.g., T cells). In some embodiments, the cells are human PBMCs and engineered (e.g., transduced) to express the CAR, or antigen-binding fragment thereof.

In some embodiments, the in vitro cytotoxicity of the engineered cells described herein (e.g., CAR-T cells) is determined. In some embodiments, the in vitro cytotoxicity of the engineered cells described herein (e.g., CAR-T cells) is determined using a Huh7 xenograft model. In some embodiments, mice treated with the engineered cells described herein (e.g., CAR-T cells) are tumor-free after 2 weeks, after 3 weeks, after 4 weeks, or after 5 weeks post-treatment.

### Recombinant Vectors

The present disclosure also provides recombinant vectors (e.g., an expression vectors) that include an isolated polynucleotide disclosed herein (e.g., a polynucleotide that encodes a polypeptide disclosed herein), host cells into which are introduced the recombinant vectors (i.e., such that the host cells contain the polynucleotide and/or a vector comprising the polynucleotide), and the production of recombinant polypeptides or fragments thereof by recombinant techniques.

A vector is a construct capable of delivering one or more polynucleotide(s) of interest to a host cell when the vector is introduced to the host cell. An “expression vector” is capable of delivering and expressing the one or more polynucleotide(s) of interest as an encoded polypeptide in a host cell into which the expression vector has been introduced. Thus, in an expression vector, the polynucleotide of interest is positioned for expression in the vector by being operably linked with regulatory elements such as a promoter, enhancer, and/or a poly-A tail, either within the vector or in the genome of the host cell at or near or flanking the integration site of the polynucleotide of interest such that the polynucleotide of interest will be translated in the host cell introduced with the expression vector.

A vector can be introduced into the host cell by methods known in the art, e.g., electroporation, chemical transfection (e.g., DEAE-dextran), transformation, transfection, and infection and/or transduction (e.g., with recombinant virus). Thus, non-limiting examples of vectors include viral vectors (which can be used to generate recombinant virus), naked DNA or RNA, plasmids, cosmids, phage vectors, and DNA or RNA expression vectors associated with cationic condensing agents.

The present disclosure provides a recombinant vector comprising a nucleic acid construct suitable for genetically modifying a cell, which can be used for treatment of pathological disease or condition.

Any vector or vector type can be used to deliver genetic material to the cell. These vectors include but are not limited to plasmid vectors, viral vectors, bacterial artificial chromosomes (BACs), yeast artificial chromosomes (YACs), and human artificial chromosomes (HACs). Viral vectors can include but are not limited to recombinant retroviral vectors, recombinant lentiviral vectors, recombinant adenoviral vectors, foamy virus vectors, recombinant adeno-associated viral (AAV) vectors, hybrid vectors, and plasmid transposons (e.g., sleeping beauty transposon system, and PiggyBac transposon system) or integrase based vector systems. Other vectors that are known in the art can also be used in connection with the methods described herein.

In some embodiments, the vector is a viral vector. The viral vector can be grown in a culture medium specific for viral vector manufacturing. Any suitable growth media and/or supplements for growing viral vectors can be used in accordance with the embodiments described herein. In some embodiments, the viral vector contains an EF1α promoter to facilitate expression.

In some embodiments, the vector used is a recombinant retroviral vector. A retroviral vector is capable of directing the expression of a nucleic acid molecule of interest. A retrovirus is present in the RNA form in its viral capsule and forms a double-stranded DNA intermediate when it replicates in the host cell. Similarly, retroviral vectors are present in both RNA and double-stranded DNA forms. The retroviral vector also includes the DNA form which contains a recombinant DNA fragment and the RNA form containing a recombinant RNA fragment. The vectors can include at least one transcriptional promoter/enhancer, or other elements which control gene expression. Such vectors can also include a packaging signal, long terminal repeats (LTRs) or portion thereof, and positive and negative strand primer binding sites appropriate to the retrovirus used. Long terminal repeats (LTRs) are identical sequences of DNA that repeat many times (e.g., hundreds or thousands of times) found at either end of retrotransposons or proviral DNA formed by reverse transcription of retroviral RNA. They are used by viruses to insert their genetic material into the host genomes. Optionally, the vectors can also include a signal which directs polyadenylation, selectable markers such as Ampicillin resistance, Neomycin resistance, TK, hygromycin resistance, phleomycin resistance histidinol resistance, or DHFR, as well as one or more restriction sites and a translation termination sequence. For example, such vectors can include a 5′ LTR, a leading sequence, a tRNA binding site, a packaging signal, an origin of second strand DNA synthesis, and a 3′ LTR or a portion thereof. Additionally, retroviral vector used herein can also refers to the recombinant vectors created by removal of the retroviral gag, pol, and env genes and replaced with the gene of interest.

In some embodiments, the vector or construct can contain a single promoter that drives the expression of one or more nucleic acid molecules. In some embodiments, such promoters can be multicistronic (bicistronic or tricistronic). For example, in some embodiments, transcription units can be engineered as a bicistronic unit containing an IRES (internal ribosome entry site), which allows coexpression of gene products (e.g. encoding CAR and an antibody or antigen binding fragment thereof) by a message from a single promoter. Alternatively, in some cases, a single promoter may direct expression of an RNA that contains, in a single open reading frame (ORF), two or three genes (e.g. encoding CAR and/or an antibody or antigen binding fragment thereof) separated from one another by sequences encoding a self-cleavage peptide (e.g., P2A or T2A) or a protease recognition site (e.g., furin). The ORF thus encodes a single polyprotein, which, either during (in the case of 2A e.g., T2A) or after translation, is cleaved into the individual proteins. In some cases, the peptide, such as T2A, can cause the ribosome to skip (ribosome skipping) synthesis of a peptide bond at the C-terminus of a 2A element, leading to separation between the end of the 2A sequence and the next peptide downstream.

Various cell lines can be used in connection with the vectors as described herein. Exemplary eukaryotic cells that may be used to express polypeptides include, but are not limited to, COS cells, including COS 7 cells; HEK293 cells, including HEK293-6E cells; CHO cells, including CHO-S, DG44. Lec13 CHO cells, and FUT8 CHO cells; PER.C6® cells; and NSO cells. In some embodiments, a particular eukaryotic host cell is selected based on its ability to make desired post-translational modifications to the antibodies or CAR molecule. For example, in some embodiments, CHO cells produce polypeptides that have a higher level of sialylation than the same polypeptide produced in HEK293 cells. In one aspect, the disclosure relates to a cell comprising the vector or the pair of vectors as described herein.

The present disclosure also provides a nucleic acid sequence comprising a nucleotide sequence encoding any of the antibodies, CAR, antigen binding fragments thereof, and/or CAR-derived binding molecules (including e.g., functional portions and functional variants thereof, polypeptides, or proteins described herein). “Nucleic acid” as used herein can include “polynucleotide,” “oligonucleotide,” and “nucleic acid molecule,” and generally means a polymer of DNA or RNA, which can be single-stranded or double-stranded, synthesized or obtained from natural sources, which can contain natural, non-natural or altered nucleotides. Furthermore, the nucleic acid comprises complementary DNA (cDNA). It is generally preferred that the nucleic acid does not comprise any insertions, deletions, inversions, and/or substitutions. However, it can be suitable in some instances, as discussed herein, for the nucleic acid to comprise one or more insertions, deletions, inversions, and/or substitutions.

The nucleic acids as described herein can be constructed based on chemical synthesis and/or enzymatic ligation reactions using procedures known in the art. For example, a nucleic acid can be chemically synthesized using naturally occurring nucleotides or variously modified nucleotides. In some of any such embodiments, the nucleotide sequence is codon-optimized.

The present disclosure also provides the nucleic acids comprising a nucleotide sequence complementary to the nucleotide sequence of any of the nucleic acids described herein or a nucleotide sequence which hybridizes under stringent conditions to the nucleotide sequence of any of the nucleic acids described herein.

In some embodiments, the nucleotide sequence encoding the CARs are separated by a peptide sequence that causes ribosome skipping. In some embodiments, the peptide that causes ribosome skipping is a P2A or T2A peptide. In some embodiments, the nucleic acid is synthetic. In some embodiments, the nucleic acid is cDNA.

In certain embodiments, the polypeptide comprises a signal peptide. In some embodiments, the signal peptide comprises a sequence that is at least at least 80%, at least 85%, at least 90%, at least 95%, or 100% identical to SEQ ID NO: 47.

The disclosure also provides a nucleic acid sequence that is at least 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, 99% identical to any nucleotide sequence as described herein, and an amino acid sequence that is at least 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, 99% identical to any amino acid sequence as described herein. In some embodiments, the disclosure relates to nucleotide sequences encoding any peptides that are described herein, or any amino acid sequences that are encoded by any nucleotide sequences as described herein.

To determine the percent identity of two amino acid sequences, or of two nucleic acid sequences, the sequences are aligned for optimal comparison purposes (e.g., gaps can be introduced in one or both of a first and a second amino acid or nucleic acid sequence for optimal alignment and non-homologous sequences can be disregarded for comparison purposes). The length of a reference sequence aligned for comparison purposes is at least 80% of the length of the reference sequence, and in some embodiments is at least 90%, 95%, or 100%. The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position. The percent identity between the two sequences is a function of the number of identical positions shared by the sequences, taking into account the number of gaps, and the length of each gap, which need to be introduced for optimal alignment of the two sequences. For example, the comparison of sequences and determination of percent identity between two sequences can be accomplished using a Blossum 62 scoring matrix with a gap penalty of 12, a gap extend penalty of 4, and a frameshift gap penalty of 5.

In some embodiments, the nucleic acid sequence is at least or about 10, 20, 30, 40, 50, 60, 70, 80, 90, 100, 110, 120, 130, 150, 200, 250, 300, 350, 400, 500, or 600 nucleotides. In some embodiments, the amino acid sequence is at least or about 5, 6, 7, 8, 9, 10, 20, 30, 40, 50, 60, 70, 80, 90, 100, 110, 120, 130, 140, 150, 160, 170, 180, 190, 200, 300, 400, 500, 600, 700, 800, or 900 amino acid residues. In some embodiments, the nucleic acid sequence is less than 10, 20, 30, 40, 50, 60, 70, 80, 90, 100, 110, 120, 130, 150, 200, 250, 300, 350, 400, 500, or 600 nucleotides. In some embodiments, the amino acid sequence is less than 5, 6, 7, 8, 9, 10, 20, 30, 40, 50, 60, 70, 80, 90, 100, 110, 120, 130, 140, 150, 160, 170, 180, 190, 200, 300, 400, 500, 600, 700, 800, or 900 amino acid residues.

### Method for Preparation of Engineered Cells

The present disclosure provides a method or process for preparing, manufacturing and/or using the engineered cells for treatment of pathological diseases or conditions.

The cells for introduction of the protein described herein, e.g., CAR, can be isolated from a sample, such as a biological sample, e.g., one obtained from or derived from a subject. In some embodiments, the subject from which the cell is isolated is one having the disease or condition or in need of a cell therapy or to which cell therapy will be administered. The subject in some embodiments is a human in need of a particular therapeutic intervention, such as the adoptive cell therapy for which cells are being isolated, processed, and/or engineered.

Accordingly, the cells in some embodiments are primary cells, e.g., primary human cells. The samples include tissue, fluid, and other samples taken directly from the subject, as well as samples resulting from one or more processing steps, such as separation, centrifugation, genetic engineering (e.g. transduction with viral vector), washing, and/or incubation. The biological sample can be a sample obtained directly from a biological source or a sample that is processed. Biological samples include, but are not limited to, body fluids, such as blood, plasma, serum, cerebrospinal fluid, synovial fluid, urine and sweat, tissue and organ samples, including processed samples derived therefrom.

In some aspects, the sample from which the cells are derived or isolated is blood or a blood-derived sample, or is or is derived from an apheresis or leukapheresis product. Exemplary samples include whole blood, peripheral blood mononuclear cells (PBMCs), leukocytes, bone marrow, thymus, tissue biopsy, tumor, leukemia, lymphoma, lymph node, gut associated lymphoid tissue, mucosa associated lymphoid tissue, spleen, other lymphoid tissues, liver, lung, stomach, intestine, colon, kidney, pancreas, breast, bone, prostate, cervix, testes, ovaries, tonsil, or other organ, and/or cells derived therefrom. Samples include, in the context of cell therapy, e.g., adoptive cell therapy, samples from autologous and allogeneic sources.

In some embodiments, the cells are derived from cell lines, e.g., T cell lines. The cells in some embodiments are obtained from a xenogeneic source, for example, from mouse, rat, or non-human primate. In some embodiments, the cells are isolated from mouse lymph nodes.

In some embodiments, the blood cells collected from the subject are washed, e.g., to remove the plasma fraction and to place the cells in an appropriate buffer or media for subsequent processing steps. In some embodiments, the cells are washed with phosphate buffered saline (PBS). In some embodiments, the wash solution lacks calcium and/or magnesium and/or many or all divalent cations. In some aspects, a washing step is accomplished a semi-automated “flow-through” centrifuge. In some aspects, a washing step is accomplished by tangential flow filtration (TFF). In some embodiments, the cells are resuspended in a variety of biocompatible buffers after washing, such as, for example, Ca²/Mg²⁺ free PBS. In certain embodiments, components of a blood cell sample are removed and the cells directly resuspended in culture media. In some embodiments, the methods include density-based cell separation methods, such as the preparation of white blood cells from peripheral blood by lysing the red blood cells and centrifugation through a Percoll or Ficoll gradient.

In some embodiments, the method comprises one or more steps of: e.g., isolating the T cells from a patient's blood; transducing the population T cells with a viral vector including the nucleic acid construct encoding a genetically engineered antigen receptor; expanding the transduced cells in vitro; and/or infusing the expanded cells into the patient, where the engineered T cells will seek and destroy antigen positive tumor cells. In some embodiments, the method further comprises: transfection of T cells with the viral vector containing the nucleic acid construct.

In some embodiments, the methods involve introducing any vectors described herein into a cell in vitro or ex vivo. In some embodiments, the vector is a viral vector and the introducing is carried out by transduction. In some embodiments, the cell is transduced for at least or about 1 hour, 2 hours, 3 hours, 4 hours, 5 hours, 6 hours, 12 hours, 18 hours, 1 day, 2 days, 3 days, 4 days, 5 days, 6 days, 1 week, 2 weeks, 3 weeks, or longer. In some embodiments, the methods further involve introducing into the cell one or more agent, wherein each of the one or more agent is independently capable of inducing a genetic disruption of a T cell receptor alpha constant (TRAC) gene and/or a T cell receptor beta constant (TRBC) gene. In some embodiments, the one or more agent is an inhibitory nucleic acid (e.g., siRNA). In some embodiments, the one or more agent is a fusion protein comprising a DNA-targeting protein and a nuclease or an RNA-guided nuclease (e.g., a clustered regularly interspaced short palindromic nucleic acid (CRISPR)-associated nuclease).

The transfection of T cells can be achieved by using any standard method such as calcium phosphate, electroporation, liposomal mediated transfer, microinjection, biolistic particle delivery system, or any other known methods by skilled artisan. In some embodiments, transfection of T cells is performed using the calcium phosphate method.

The present disclosure provides a method to create a personalized anti-tumor immunotherapy. Genetically engineered T cells can be produced from a patient's blood cells. These engineered T cells are then reinfused into the patient as a cellular therapy product.

### Methods of Treatment

The antibodies and antigen-binding fragments thereof, CARs, and immune cells disclosed herein can be used for various therapeutic purposes. In one aspect, the disclosure provides methods for treating a cancer in a subject, methods of reducing the rate of the increase of volume of a tumor in a subject over time, methods of reducing the risk of developing a metastasis, or methods of reducing the risk of developing an additional metastasis in a subject. In some embodiments, the treatment can halt, slow, retard, or inhibit progression of a cancer. In some embodiments, the treatment can result in the reduction of in the number, severity, and/or duration of one or more symptoms of the cancer in a subject.

In one aspect, the disclosure features methods that include administering a therapeutically effective amount of antibodies or antigen binding fragments thereof, or engineered cells expressing CAR, to a subject in need thereof (e.g., a subject having, or identified or diagnosed as having, a cancer).

In some embodiments, the subject has GPC3-positive cancer. In some embodiments, the subject has liver cancer (e.g., hepatocellular carcinoma), glioma, lung cancer, colorectal cancer, head and neck cancer, stomach cancer, renal cancer, urothelial cancer, testis cancer, breast cancer, cervical cancer, endometrial cancer, and/or ovarian cancer. In some embodiments, the subject has squamous cell lung carcinoma, or solid tumor. In some embodiments, the subject has a CNS tumor, thyroid cancer, gastrointestinal cancer, skin cancer, sarcoma, urogenital cancer, and/or germ cell tumor.

In some embodiments, the compositions and methods disclosed herein can be used for treatment of patients at risk for a cancer. Patients with cancer can be identified with various methods known in the art.

As used herein, by an “effective amount” is meant an amount or dosage sufficient to effect beneficial or desired results including halting, slowing, retarding, or inhibiting progression of a disease, e.g., a cancer. An effective amount will vary depending upon, e.g., an age and a body weight of a subject to which the therapeutic agent and/or therapeutic compositions is to be administered, a severity of symptoms and a route of administration, and thus administration can be determined on an individual basis.

As used herein, the term “delaying development of a disease” refers to defer, hinder, slow, retard, stabilize, suppress and/or postpone development of the disease (such as cancer). This delay can be of varying lengths of time, depending on the history of the disease and/or individual being treated. As is evident to one skilled in the art, a sufficient or significant delay can, in effect, encompass prevention, in that the individual does not develop the disease. For example, a late stage cancer, such as development of metastasis, can be delayed.

An effective amount can be administered in one or more administrations. By way of example, an effective amount of a composition is an amount sufficient to ameliorate, stop, stabilize, reverse, inhibit, slow and/or delay progression of a cancer in a patient or is an amount sufficient to ameliorate, stop, stabilize, reverse, slow and/or delay proliferation of a cell (e.g., a biopsied cell, any of the cancer cells described herein, or cell line (e.g., a cancer cell line)) in vitro. As is understood in the art, an effective may vary, depending on, inter alia, patient history as well as other factors such as the type (and/or dosage) of compositions used.

Effective amounts and schedules for administrations may be determined empirically, and making such determinations is within the skill in the art. Those skilled in the art will understand that the dosage that must be administered will vary depending on, for example, the mammal that will receive the treatment, the route of administration, the particular type of therapeutic agents and other drugs being administered to the mammal. Guidance in selecting appropriate doses can be found in the literature. In addition, a treatment does not necessarily result in the 100% or complete treatment or prevention of a disease or a condition. There are multiple treatment/prevention methods available with a varying degree of therapeutic effect which one of ordinary skill in the art recognizes as a potentially advantageous therapeutic mean.

In some embodiments, treating the subject using the CAR-T cells disclosed herein results in at least 20%, 25%, 30%, 35%, 40%, 45%, 50%, 60%, 70%, 80%, 85%, 90%, 95% or 100% tumor volume reduction in the subject. In some embodiments, the subject is tumor free after 2-3 weeks post treatment.

In some aspects, the present disclosure also provides methods of diagnosing a disease/condition in a mammal, wherein the CARs, antibodies, or antigen binding fragments, interact with the sample(s) obtained from a subject to form a complex, wherein the sample can comprise one more cells, polypeptides, proteins, nucleic acids, antibodies, or antigen binding portions, blood, whole cells, lysates thereof, or a fraction of the whole cell lysates, e.g., a nuclear or cytoplasmic fraction, a whole protein fraction, or a nucleic acid fraction thereof, wherein the detection of the complex is the indicative of presence of a condition in the mammal, wherein the condition is cancer or infection. Further, the detection of the complex can be in any number of way known in the art but not limited to, ELISA, Flow cytometery, Fluorescence in situ hybridization (FISH), Polymerase chain reaction (PCR), microarray, southern blotting, electrophoresis, Phage analysis, chromatography and more. Thus, the treatment methods can further include determining whether a subject can benefit from a treatment as disclosed herein, e.g., by determining whether the subject has infection or cancer.

In any of the methods described herein, the engineered cells, optionally with at least one additional therapeutic agent, can be administered to the subject at least once a week (e.g., once a week, twice a week, three times a week, four times a week, once a day, twice a day, or three times a day). In some embodiments, at least two different engineered cells (e.g., cells expressing different CARs) are administered in the same composition (e.g., a liquid composition). In some embodiments, engineered cells and at least one additional therapeutic agent are administered in the same composition (e.g., a liquid composition). In some embodiments, engineered cells and at least one additional therapeutic agent are administered in two different compositions. In some embodiments, the at least one additional therapeutic agent is administered as a pill, tablet, or capsule. In some embodiments, the at least one additional therapeutic agent is administered in a sustained-release oral formulation. In some embodiments, the one or more additional therapeutic agents can be administered to the subject prior to, concurrently with, or after administering the engineered cells to the subject.

In some embodiments, the additional therapeutic agent can comprise one or more inhibitors selected from the group consisting of an inhibitor of B-Raf, an EGFR inhibitor, an inhibitor of a MEK, an inhibitor of ERK, an inhibitor of K-Ras, an inhibitor of c-Met, an inhibitor of anaplastic lymphoma kinase (ALK), an inhibitor of a phosphatidylinositol 3-kinase (PI3K), an inhibitor of an Akt, an inhibitor of mTOR, a dual PI3K/mTOR inhibitor, an inhibitor of Bruton's tyrosine kinase (BTK), and an inhibitor of Isocitrate dehydrogenase 1 (IDH1) and/or Isocitrate dehydrogenase 2 (IDH2). In some embodiments, the additional therapeutic agent is an inhibitor of indoleamine 2, 3-dioxygenase-1) (IDO1) (e.g., epacadostat). In some embodiments, the additional therapeutic agent can comprise one or more inhibitors selected from the group consisting of an inhibitor of HER3, an inhibitor of LSD1, an inhibitor of MDM2, an inhibitor of BCL2, an inhibitor of CHK1, an inhibitor of activated hedgehog signaling pathway, and an agent that selectively degrades the estrogen receptor.

In some embodiments, the additional therapeutic agent can comprise one or more therapeutic agents selected from the group consisting of Trabectedin, nab-paclitaxel, Trebananib, Pazopanib, Cediranib, Palbociclib, everolimus, fluoropyrimidine, IFL, regorafenib, Reolysin, Alimta, Zykadia, Sutent, temsirolimus, axitinib, everolimus, sorafenib, Votrient, Pazopanib, IMA-901, AGS-003, cabozantinib, Vinflunine, an Hsp90 inhibitor, Ad-GM-CSF, Temazolomide, IL-2, IFNa, vinblastine, Thalomid, dacarbazine, cyclophosphamide, lenalidomide, azacytidine, lenalidomide, bortezomid, amrubicine, carfilzomib, pralatrexate, and enzastaurin.

In some embodiments, the additional therapeutic agent can comprise one or more therapeutic agents selected from the group consisting of an adjuvant, a TLR agonist, tumor necrosis factor (TNF) alpha, IL-1, HMGB1, an IL-10 antagonist, an IL-4 antagonist, an IL-13 antagonist, an IL-17 antagonist, an HVEM antagonist, an ICOS agonist, a treatment targeting CX₃CL1, a treatment targeting CXCL9, a treatment targeting CXCL10, a treatment targeting CCL5, an LFA-1 agonist, an ICAM1 agonist, and a Selectin agonist.

In some embodiments, carboplatin, nab-paclitaxel, paclitaxel, cisplatin, pemetrexed, gemcitabine, FOLFOX, or FOLFIRI are administered to the subject. In some embodiments, the additional therapeutic agent is selected from asparaginase, busulfan, carboplatin, cisplatin, daunorubicin, doxorubicin, fluorouracil, gemcitabine, hydroxyurea, methotrexate, paclitaxel, rituximab, vinblastine, vincristine and/or combinations thereof.

### Compositions and Formulations

The present disclosure provides compositions (including pharmaceutical and therapeutic compositions) containing the engineered cells and populations thereof, produced by the methods disclosed herein. Also provided are methods, e.g., therapeutic methods for administrating the engineered cells and compositions thereof to subjects, e.g., patients or animal models (e.g., mice).

Compositions including the engineered cells for administration, including pharmaceutical compositions and formulations, such as unit dose form compositions including the number of cells for administration in a given dose or fraction thereof are provided. The pharmaceutical compositions and formulations can include one or more optional pharmaceutically acceptable carrier or excipient. In some embodiments, the composition includes at least one additional therapeutic agent.

A pharmaceutically acceptable carrier refers to an ingredient in a pharmaceutical composition, other than an active ingredient. The pharmaceutically acceptable carrier does not interfere with the active ingredient and is nontoxic to a subject. A pharmaceutically acceptable carrier can include, but is not limited to, a buffer, excipient, stabilizer, or preservative. The pharmaceutical formulation refers to process in which different substances and/or agents are combined to produce a final medicinal product. The formulation studies involve developing a preparation of drug acceptable for patient. Additionally, a preparation which is in such form as to permit the biological activity of an active ingredient contained therein to be effective, and which contains no additional components which are unacceptably toxic to a subject to which the formulation would be administered.

In some embodiments, the choice of carrier is determined in part by the particular cell (e.g., T cell or NK cell) and/or by the method of administration. A variety of suitable formulations are available. For example, the pharmaceutical composition can contain preservatives. Suitable preservatives can include, for example, methylparaben, propylparaben, sodium benzoate, and benzalkonium chloride. In some embodiments, a mixture of two or more preservatives is used. The preservative or mixtures thereof are typically present in an amount of about 0.0001% to about 2% by weight of the total composition. Carriers are described, e.g., by Remington's Pharmaceutical Sciences 16th edition, Osol, A. Ed. (1980). Pharmaceutically acceptable carriers are generally nontoxic to recipients at the dosages and concentrations employed, and include, but are not limited to: buffers such as phosphate, citrate, and other organic acids; antioxidants including ascorbic acid and methionine; preservatives (such as octadecyldimethylbenzyl ammonium chloride; hexamethonium chloride; benzalkonium chloride; benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g. Zn-protein complexes); and/or non-ionic surfactants such as polyethylene glycol (PEG).

Suitable buffering agents include, for example, citric acid, sodium citrate, phosphoric acid, potassium phosphate, and various other acids and salts. In some embodiments, a mixture of two or more buffering agents is used. The buffering agent or mixtures thereof are typically present in an amount of about 0.001% to about 4% by weight of the total composition. Methods for preparing administrable pharmaceutical compositions are known. Exemplary methods are described in more detail in, for example, Remington: The Science and Practice of Pharmacy, Lippincott Williams & Wilkins; 21st ed. (May 1, 2005).

The formulations can include aqueous solutions. The formulation or composition can also contain more than one active ingredient useful for a particular indication, disease, or condition being treated with the engineered cells, preferably those with activities complementary to the cells, where the respective activities do not adversely affect one another. Such active ingredients are suitably present in combination in amounts that are effective for the purpose intended. Thus, in some embodiments, the pharmaceutical composition can further include other pharmaceutically active agents or drugs, such as checkpoint inhibitors, fusion proteins, chemotherapeutic agents, e.g., asparaginase, busulfan, carboplatin, cisplatin, daunorubicin, doxorubicin, fluorouracil, gemcitabine, hydroxyurea, methotrexate, paclitaxel, rituximab, vinblastine, and/orvincristine.

The pharmaceutical composition in some embodiments contains the cells in amounts effective to treat or prevent the disease or condition, such as a therapeutically effective or prophylactically effective amount. Therapeutic or prophylactic efficacy in some embodiments is monitored by periodic assessment of treated subjects. The desired dosage can be delivered by a single bolus administration of the cells, by multiple bolus administrations of the cells, or by continuous infusion administration of the cells.

The cells and compositions can be administered using standard administration techniques, formulations, and/or devices. Administration of the cells can be autologous or heterologous. For example, immunoresponsive T cells or progenitors can be obtained from one subject, and administered to the same subject or a different, compatible subject after genetically modifying them in accordance with various embodiments described herein. Peripheral blood derived immunoresponsive T cells or their progeny (e.g., in vivo, ex vivo or in vitro derived) can be administered via localized injection, including catheter administration, systemic injection, localized injection, intravenous injection, or parenteral administration. Usually, when administering a therapeutic composition (e.g., a pharmaceutical composition containing a genetically modified immunoresponsive cell), it is generally formulated in a unit dosage injectable form (solution, suspension, emulsion).

Formulations disclosed herein include those for oral, intravenous, intraperitoneal, subcutaneous, pulmonary, transdermal, intramuscular, intranasal, buccal, sublingual, or suppository administration. In some embodiments, the cell populations are administered parenterally. The term “parenteral,” as used herein, includes intravenous, intramuscular, subcutaneous, rectal, vaginal, and intraperitoneal administration. In some embodiments, the cells are administered to the subject using peripheral systemic delivery by intravenous, intraperitoneal, or subcutaneous injection.

Sterile injectable solutions can be prepared by incorporating the cells in a solvent, such as in admixture with a suitable carrier, diluent, or excipient such as sterile water, physiological saline, glucose, dextrose, or the like. The compositions can contain auxiliary substances such as wetting, dispersing, or emulsifying agents (e.g., methylcellulose), pH buffering agents, gelling or viscosity enhancing additives, preservatives, flavoring agents, and/or colors, depending upon the route of administration and the preparation desired. Standard texts can in some aspects be consulted to prepare suitable preparations.

Various additives which enhance the stability and sterility of the compositions, including antimicrobial preservatives, antioxidants, chelating agents, and buffers, can be added. Prevention of the action of microorganisms can be ensured by various antibacterial and antifungal agents, for example, parabens, chlorobutanol, phenol, and sorbic acid. Prolonged absorption of the injectable pharmaceutical form can be brought about by the use of agents delaying absorption, for example, aluminum monostearate and gelatin.

The formulations to be used for in vivo administration are generally sterile. Sterility can be readily accomplished, e.g., by filtration through sterile filtrationmembranes.

The compositions or pharmaceutical compositions as described herein can be included in a container, pack, or dispenser together with instructions for administration.

### Methods of Administration

Provided are also methods of administering the cells, populations, and compositions, and uses of such cells, populations, and compositions to treat or prevent diseases, conditions, and disorders, including cancers. In some embodiments, the methods described herein can reduce the risk of the developing diseases, conditions, and disorders as described herein.

In some embodiments, the cells, populations, and compositions, described herein are administered to a subject or patient having a particular disease or condition to be treated, e.g., via adoptive cell therapy, such as adoptive T cell therapy. In some embodiments, cells and compositions prepared by the provided methods, such as engineered compositions and end-of-production compositions following incubation and/or other processing steps, are administered to a subject, such as a subject having or at risk for the disease or condition. In some aspects, the methods thereby treat, e.g., ameliorate one or more symptom of, the disease or condition, such as by lessening tumor burden in cancer expressing an antigen recognized by the engineered T cells.

Methods for administration of cells for adoptive cell therapy are known and can be used in connection with the provided methods and compositions. For example, adoptive T cell therapy methods are described, e.g., in U.S. 2003/0170238; U.S. Pat. No. 4,690,915; Rosenberg, Nature reviews Clinical oncology 8.10 (2011): 577; Themeli et al., Nature biotechnology 31.10 (2013): 928; Tsukahara et al., Biochemical and biophysical research communications 438.1 (2013): 84-89; Davila et al., PloS one 8.4 (2013); each of which is incorporated herein by reference in its entirety.

In some embodiments, the cell therapy, e.g., adoptive T cell therapy, is carried out by autologous transfer, in which the T cells are isolated and/or otherwise prepared from the subject who is to receive the cell therapy, or from a sample derived from such a subject. Thus, in some aspects, the cells are derived from a subject, e.g., patient, in need of a treatment and the cells, following isolation and processing are administered to the same subject.

In some embodiments, the cell therapy, e.g., adoptive T cell therapy, is carried out by allogeneic transfer, in which the T cells are isolated and/or otherwise prepared from a subject other than a subject who is to receive or who ultimately receives the cell therapy, e.g., a first subject. In such embodiments, the cells then are administered to a different subject, e.g., a second subject, of the same species. In some embodiments, the first and second subjects are genetically identical. In some embodiments, the first and second subjects are genetically similar. In some embodiments, the second subject expresses the same HLA class or supertype as the first subject.

In some embodiments, the subject has been treated with a therapeutic agent targeting the disease or condition, e.g. the tumor, prior to administration of the cells or composition containing the cells. In some aspects, the subject is refractory or non-responsive to the other therapeutic agent. In some embodiments, the subject has persistent or relapsed disease, e.g., following treatment with another therapeutic intervention, including chemotherapy, radiation, and/or hematopoietic stem cell transplantation (HSCT), e.g., allogenic HSCT. In some embodiments, the administration effectively treats the subject despite the subject having become resistant to another therapy.

In some embodiments, the subject is responsive to the other therapeutic agent, and treatment with the therapeutic agent reduces disease burden. In some aspects, the subject is initially responsive to the therapeutic agent, but exhibits a relapse of the disease or condition over time. In some embodiments, the subject has not relapsed. In some such embodiments, the subject is determined to be at risk for relapse, such as at high risk of relapse, and thus the cells are administered prophylactically, e.g., to reduce the likelihood of or prevent relapse. In some embodiments, the subject has not received prior treatment with another therapeutic agent.

In some embodiments, the cells are administered at a desired dosage, which in some aspects includes a desired dose or number of cells or cell type(s) and/or a desired ratio of cell types. Thus, the dosage of cells in some embodiments is based on a total number of cells (or number per kg body weight) and a desired ratio of the individual populations or sub-types, such as the CD4+ to CD8+ ratio. In some embodiments, the dosage of cells is based on a desired total number (or number per kg of body weight) of cells in the individual populations or of individual cell types. In some embodiments, the dosage is based on a combination of such features, such as a desired number of total cells, desired ratio, and desired total number of cells in the individual populations.

In some embodiments, the populations or sub-types of cells, such as CD8+ and CD4+ T cells, are administered at or within a tolerated difference of a desired dose of total cells, such as a desired dose of T cells. In some embodiments, the desired dose is a desired number of cells or a desired number of cells per unit of body weight of the subject to whom the cells are administered, e.g., cells/kg. In some embodiments, the desired dose is at or above a minimum number of cells or minimum number of cells per unit of body weight. In some embodiments, among the total cells, administered at the desired dose, the individual populations or sub-types are present at or near a desired output ratio (such as CD4+ to CD8+ ratio), e.g., within a certain tolerated difference or error of such a ratio.

In some embodiments, the cells are administered at or within a tolerated difference of a desired dose of one or more of the individual populations or sub-types of cells, such as a desired dose of CD4+ cells and/or a desired dose of CD8+ cells. In some embodiments, the desired dose is a desired number of cells of the sub-type or population, or a desired number of such cells per unit of body weight of the subject to whom the cells are administered, e.g., cells/kg. In some embodiments, the desired dose is at or above a minimum number of cells of the population or sub-type, or minimum number of cells of the population or sub-type per unit of body weight.

Thus, in some embodiments, the dosage is based on a desired fixed dose of total cells and a desired ratio, and/or based on a desired fixed dose of one or more, e.g., each, of the individual sub-types or sub-populations. Thus, in some embodiments, the dosage is based on a desired fixed or minimum dose of T cells and a desired ratio of CD4+ to CD8+ cells, and/or is based on a desired fixed or minimum dose of CD4+ and/or CD8+ cells.

In certain embodiments, the cells or individual populations of sub-types of cells, are administered to the subject at a range of about one million to about 100 billion cells, such as, e.g., 1 million to about 50 billion cells (e.g., about 5 million cells, about 25 million cells, about 500 million cells, about 1 billion cells, about 5 billion cells, about 20 billion cells, about 30 billion cells, about 40 billion cells, or a range defined by any two of the foregoing values), such as about 10 million to about 100 billion cells (e.g., about 20 million cells, about 30 million cells, about 40 million cells, about 60 million cells, about 70 million cells, about 80 million cells, about 90 million cells, about 10 billion cells, about 25 billion cells, about 50 billion cells, about 75 billion cells, about 90 billion cells, or a range defined by any two of the foregoing values), and in some cases about 100 million cells to about 50 billion cells (e.g., about 120 million cells, about 250 million cells, about 350 million cells, about 450 million cells, about 650 million cells, about 800 million cells, about 900 million cells, about 3 billion cells, about 30 billion cells, about 45 billion cells) or any value in between these ranges.

In some embodiments, the dose of total cells and/or dose of individual sub-populations of cells is within a range of between at or about 10⁴ and at or about 10⁹ cells/kilograms (kg) body weight, such as between 10⁵ and 10⁶ cells/kg body weight, for example, at least or at least about or at or about 1×10⁵ cells/kg, 1.5×10⁵ cells/kg, 2×10⁵ cells/kg, or 1×10⁶ cells/kg body weight.

For example, in some embodiments, the cells are administered at, or within a certain range of error of, between at or about 10⁴ and at or about 10⁹ T cells/kilograms (kg) body weight, such as between 10⁵ and 10⁶ T cells/kg body weight, for example, at least or at least about or at or about 1×10⁵ T cells/kg, 1.5×10⁵ T cells/kg, 2×10⁵ T cells/kg, or 1×10⁶ T cells/kg body weight.

In some embodiments, the cells are administered at or within a certain range of error of between at or about 10⁴ and at or about 10⁹ CD4+ and/or CD8+ cells/kilograms (kg) body weight, such as between 10⁵ and 10⁶ CD4+ and/or CD8+ cells/kg body weight, for example, at least or at least about or at or about 1×10⁵ CD4+ and/or CD8+ cells/kg, 1.5×10⁵ CD4+ and/or CD8+ cells/kg, 2×10⁵ CD4+ and/or CD8+ cells/kg, or 1×10⁶ CD4+ and/or CD8+ cells/kg body weight.

In some embodiments, the cells are administered at or within a certain range of error of, greater than, and/or at least about 1×10⁶, about 2.5×10⁶, about 5×10⁶, about 7.5×10⁶, or about 9×10⁶ CD4+ cells, and/or at least about 1×10⁶, about 2.5×10⁶, about 5×10⁶, about 7.5×10⁶, or about 9×10⁶ CD8+ cells, and/or at least about 1×10⁶, about 2.5×10⁶, about 5×10⁶, about 7.5×10⁶, or about 9×10⁶ T cells. In some embodiments, the cells are administered at or within a certain range of error of between about 10⁸ and 10¹² or between about 10¹⁰ and 10¹¹ cells, between about 10⁸ and 10¹² or between about 10¹⁰ and 10¹¹ CD4+ cells, and/or between about 10⁸ and 10¹² or between about 10¹⁰ and 10¹¹ CD8+ cells.

In some embodiments, the cells are administered at or within a tolerated range of a desired output ratio of multiple cell populations or sub-types, such as CD4+ and CD8+ cells or sub-types. In some aspects, the desired ratio can be a specific ratio or can be a range of ratios, for example, in some embodiments, the desired ratio (e.g., ratio of CD4+ to CD8+ cells) is between at or about 1:5 and at or about 5:1 (or greater than about 1:5 and less than about 5:1), or between at or about 1:3 and at or about 3:1 (or greater than about 1:3 and less than about 3:1), such as between at or about 2:1 and at or about 1:5 (or greater than about 1:5 and less than about 2:1, such as at or about 5:1, 4.5:1, 4:1, 3.5:1, 3:1, 2.5:1, 2:1, 1.9:1, 1.8:1, 1.7:1, 1.6:1, 1.5:1, 1.4:1, 1.3:1, 1.2:1, 1.1:1, 1:1, 1:1.1, 1:1.2, 1:1.3, 1:1.4, 1:1.5, 1:1.6, 1:1.7, 1:1.8, 1:1.9:1:2, 1:2.5, 1:3, 1:3.5, 1:4, 1:4.5, or 1:5. In some aspects, the tolerated difference is within about 1%, about 2%, about 3%, about 4% about 5%, about 10%, about 15%, about 20%, about 25%, about 30%, about 35%, about 40%, about 45%, about 50% of the desired ratio, including any value in between these ranges. In some aspects, the CAR described here provides improved expression and activity, thereby providing therapeutic effects even at a low effector to target (E:T) ratio.

Optimal response to therapy can depend on the ability of the engineered recombinant receptors such as CARs, to be consistently and reliably expressed on the surface of the cells and/or bind the target antigen. For example, in some cases, properties of certain recombinant receptors, e.g., CARs, can affect the expression and/or activity of the recombinant receptor, in some cases when expressed in a cell, such as a human T cell, used in cell therapy. In some contexts, the level of expression of particular recombinant receptors, e.g., CARs, can be low, and activity of the engineered cells, such as human T cells, expressing such recombinant receptors, may be limited due to poor expression or poor signaling activity. In some cases, consistency and/or efficiency of expression of the recombinant receptor, and activity of the receptor is limited in certain cells or certain cell populations of available therapeutic approaches. In some cases, a large number of engineered T cells (a high effector to target (E:T) ratio) is required to exhibit functional activity. In some embodiments, the desired ratio (E:T ratio) is between at or about 1:10 and at or about 10:1 (or greater than about 1:10 and less than about 10:1), or between at or about 1:1 and at or about 10:1 (or greater than about 1:1 and less than about 5:1), such as between at or about 2:1 and at or about 10:1. In some embodiments, the E:T ratio is greater than or about 1:1, 2:1, 3:1, 4:1, 5:1, 6:1, 7:1, 8:1, 9:1, or 10:1. In some embodiments, the E:T ratio is about 3:1, about 1:1, or about 0.3:1.

For the prevention or treatment of disease, the appropriate dosage may depend on the type of disease to be treated, the type of cells or recombinant receptors, the severity and course of the disease, whether the cells are administered for preventive or therapeutic purposes, previous therapy, the subject's clinical history and response to the cells, and the discretion of the attending physician. The compositions and cells are in some embodiments suitably administered to the subject at one time or over a series of treatments.

The cells described herein can be administered by any suitable means, for example, by bolus infusion, by injection, e.g., intravenous or subcutaneous injections, intraocular injection, periocular injection, subretinal injection, intravitreal injection, trans-septal injection, subscleral injection, intrachoroidal injection, intracameral injection, subconjectval injection, subconjuntival injection, sub-Tenon's injection, retrobulbar injection, peribulbar injection, or posterior juxtascleral delivery. In some embodiments, they are administered by parenteral, intrapulmonary, and intranasal, and, if desired for local treatment, intralesional administration. Parenteral infusions include intramuscular, intravenous, intraarterial, intraperitoneal, or subcutaneous administration. In some embodiments, a given dose is administered by a single bolus administration of the cells. In some embodiments, it is administered by multiple bolus administrations of the cells, for example, over a period of no more than 3 days, or by continuous infusion administration of the cells.

In some embodiments, the cells are administered as part of a combination treatment, such as simultaneously with or sequentially with, in any order, another therapeutic intervention, such as an antibody or engineered cell or receptor or agent, such as a cytotoxic or therapeutic agent. The cells in some embodiments are co-administered with one or more additional therapeutic agents or in connection with another therapeutic intervention, either simultaneously or sequentially in any order. In some contexts, the cells are co-administered with another therapy sufficiently close in time such that the cell populations enhance the effect of one or more additional therapeutic agents, or vice versa. In some embodiments, the cells are administered prior to the one or more additional therapeutic agents. In some embodiments, the cells are administered after the one or more additional therapeutic agents. In some embodiments, the one or more additional agents includes a cytokine, such as IL-2, for example, to enhance persistence. In some embodiments, the methods comprise administration of a chemotherapeutic agent.

Following administration of the cells, the biological activity of the engineered cell populations in some embodiments is measured, e.g., by any of a number of known methods. Parameters to assess include specific binding of engineered T cells to the antigen, in vivo, e.g., by imaging, or ex vivo, e.g., by ELISA or flow cytometry. In certain embodiments, the ability of the engineered cells to destroy target cells can be measured using any suitable method known in the art, such as cytotoxicity assays described in, for example, Kochenderfer et al., “Construction and pre-clinical evaluation of an anti-CD19 chimeric antigen receptor.” Journal of immunotherapy (Hagerstown, Md.: 1997) 32.7 (2009): 689 and Hermans et al., “The VITAL assay: a versatile fluorometric technique for assessing CTL- and NKT-mediated cytotoxicity against multiple targets in vitro and in vivo.” Journal of immunological methods 285.1 (2004): 25-40. In certain embodiments, the biological activity of the cells is measured by assaying expression and/or secretion of one or more cytokines, such as CD107a, IFNγ, IL-2, and TNF. In some aspects the biological activity is measured by assessing clinical outcome, such as reduction in tumor burden or load.

Repeated dosing methods are provided in which a first dose of cells is given followed by one or more second consecutive doses. The timing and size of the multiple doses of cells generally are designed to increase the efficacy and/or activity and/or function of engineered cells as described herein, when administered to a subject in adoptive therapy methods. The methods involve administering a first dose, generally followed by one or more consecutive doses, with particular time frames between the different doses.

In the context of adoptive cell therapy, administration of a given “dose” encompasses administration of the given amount or number of cells as a single composition and/or single uninterrupted administration, e.g., as a single injection or continuous infusion, and also encompasses administration of the given amount or number of cells as a split dose, provided in multiple individual compositions or infusions, over a specified period of time (e.g., no more than 3 days). Thus, in some contexts, the first or consecutive dose is a single or continuous administration of the specified number of cells, given or initiated at a single point in time. In some contexts, however, the first or consecutive dose is administered in multiple injections or infusions over a limited time period (e.g., no more than three days), such as once a day for three days or for two days or by multiple infusions over a single day period.

The cells of the first dose are administered in a single pharmaceutical composition. In some embodiments, the cells of the consecutive dose are administered in a single pharmaceutical composition.

In some embodiments, the cells of the first dose are administered in a plurality of compositions, collectively containing the cells of the first dose. In some embodiments, the cells of the consecutive dose are administered in a plurality of compositions, collectively containing the cells of the consecutive dose. In some aspects, additional consecutive doses can be administered in a plurality of compositions over a period of no more than 3 days.

With reference to a prior dose, such as a first dose, the term “consecutive dose” refers to a dose that is administered to the same subject after the prior, e.g., first, dose without any intervening doses having been administered to the subject in the interim. Nonetheless, the term does not encompass the second, third, and/or so forth, injection or infusion in a series of infusions or injections comprised within a single split dose. Thus, unless otherwise specified, a second infusion within a one, two or three-day period is not considered to be a “consecutive” dose as used herein. Likewise, a second, third, and so-forth in the series of multiple doses within a split dose also is not considered to be an “intervening” dose in the context of the meaning of “consecutive” dose. Thus, unless otherwise specified, a dose administered a certain period of time, greater than three days, after the initiation of a first or prior dose, is considered to be a “consecutive” dose even if the subject receives a second or subsequent injection or infusion of the cells following the initiation of the first dose, so long as the second or subsequent injection or infusion occurred within the three-day period following the initiation of the first or priordose.

Thus, unless otherwise specified, multiple administrations of the same cells over a period of up to 3 days is considered to be a single dose, and administration of cells within 3 days of an initial administration is not considered a consecutive dose and is not considered to be an intervening dose for purposes of determining whether a second dose is “consecutive” to the first.

In some embodiments, multiple consecutive doses are given, in some aspects using the same timing guidelines as those with respect to the timing between the first dose and first consecutive dose, e.g., by administering a first and multiple consecutive doses.

In some embodiments, the timing between the first dose and first consecutive dose, or a first and multiple consecutive doses, is such that each consecutive dose is given within a period of time is greater than about 5 days, 6 days, 7 days, 8 days, 9 days, 10 days, 11 days, 12 days, 13 days, 14 days, 15 days, 16 days, 17 days, 18 days, 19 days, 20 days, 21 days, 22 days, 23 days, 24 days, 25 days, 26 days, 27 days, 28 days or more. In some embodiments, the consecutive dose is given within a time period that is less than about 28 days after the administration of the first or immediately prior dose. The additional multiple additional consecutive dose or doses also are referred to as subsequent dose or subsequent consecutive dose.

The size of the first and/or one or more consecutive doses of cells are generally designed to provide improved efficacy and/or reduced risk of toxicity. In some aspects, a dosage amount or size of a first dose or any consecutive dose is any dosage or amount as described above. In some embodiments, the number of cells in the first dose or in any consecutive dose is between about 0.5×10⁶ cells/kg body weight of the subject and 5×10⁶ cells/kg, between about 0.75×10⁶ cells/kg and 3×10⁶ cells/kg or between about 1×10⁶ cells/kg and 2×10⁶ cells/kg.

As used herein, “first dose” is used to describe the timing of a given dose being prior to the administration of a consecutive or subsequent dose. The term does not necessarily imply that the subject has never before received a dose of cell therapy or even that the subject has not before received a dose of the same cells or cells expressing the same recombinant receptor or targeting the same antigen.

In some embodiments, multiple doses can be administered to a subject over an extended period of time (e.g., over a period of at least 1 week, 2 weeks, 3 weeks, 1 month, 2 months, 3 months, 4 months, 5 months, 6 months, 7 months, 8 months, 9 months, 10 months, 11 months, 12 months, 1 year, 2 years, 3 years, 4 years, or 5 years). A skilled medical professional may determine the length of the treatment period using any of the methods described herein for diagnosing or following the effectiveness of treatment (e.g., the observation of at least one symptom of cancer).

### EXAMPLES

The disclosure is further described in the following examples, which do not limit the scope of the disclosure described in the claims.

### Example 1: Humanization of Anti-GPC3 Murine Antibody GC33

An example for generating humanized VH and VL of the anti-GPC3 GC33 scFv (SEQ ID NO: 2) using CDR grafting technology is provided.

First, human antibody germlines with highest sequence homology to GC33 variable domains were identified as HV1-46 for VH and KV2-29 for VL. The murine VH and VL CDRs of GC33 were straightly grafted to these human frameworks (Gene Bank accession #ABF83374 for VH and QEP23526 for VL), yielding a humanized VH1 (SEQ ID NO: 3) and VL1 (SEQ ID NO: 4). In order to restore the binding activity, back mutations in the framework regions, M70L, R72A and A97T were introduced to the straightly-grafted VH, yielding a humanized VH2 sequence (SEQ ID NO: 5). To increase the humanization of CDRs, several amino acid residues in VH1 and VL1 were mutated to their human counterparts in HV1-46 and KV2-29 germlines, respectively. These humanized variable domains were VH2.M1 with D31S mutation in HCDR1 (SEQ ID NO: 6), VH2.M5 with K65Q mutation in HCDR2 (SEQ ID NO: 7), VH2.M6 with both HCDR mutations (SEQ ID NO: 8) and VL1.M2 with N58S mutation in LCDR2 (SEQ ID NO: 9).

The humanized VLs (VL1 and VL1.M2) were connected to humanized VHs (VH2, VH2.M1, VH2.M5 and VH2.M6) with a (G4S)₃ linker (SEQ ID NO: 10) to construct humanized scFvs. These scFvs were expressed with a C-terminal Fc tag in HEK293 cells. The crude scFv proteins secreted to the cell culture supernatant were used for binding affinity determination using surface plasmon resonance (SPR) on a Biacore T200 machine. Briefly, the scFv protein was captured on the sensor chip pre-coated with goat-anti-human Fc polyclonal antibody (pAb) (Jackson ImmunoResearch, cat #109-005-098) via the interaction between pAb and the Fc tag, then the antigen GPC3 concentration ranging from 10 nM to 320 nM flowed through the sensor chip. After 100 s of association, running buffer (10 mM HEPES, 150 mM NaCl, 3 mM EDTA, 0.005% Tween-20, pH 7.4) flowed through the sensor chip to allow the scFv-GPC3 complex to dissociation. The sensor chip was regenerated using 50 mM HCl between cycles. The binding affinity of humanized scFvs is comparable to that of mouse scFv (Table 1).

TABLE 1
Binding affinity of mouse and humanized scFvs.

| | Species | kₐ (1/Ms) | k_{d} (1/s) | K_{D} (M) |
|---|---|---|---|---|
| VL-VH | Mouse | 1.3E+05 | 2.3E−04 | 1.8E−09 |
| VL1-VH2 | Humanized | 1.7E+05 | 2.9E−04 | 1.7E−09 |
| VL1-VH2.M1 | Humanized | 1.9E+05 | 3.6E−04 | 1.9E−09 |
| VL1-VH2.M5 | Humanized | 1.7E+05 | 2.7E−04 | 1.6E−09 |
| VL1-VH2.M6 | Humanized | 1.9E+05 | 3.7E−04 | 2.0E−09 |
| VL1.M2-VH2 | Humanized | 1.6E+05 | 3.5E−04 | 2.2E−09 |
| VL1.M2-VH2.M1 | Humanized | 1.7E+05 | 5.1E−04 | 3.0E−09 |
| VL1.M2-VH2.M5 | Humanized | 1.7E+05 | 3.7E−04 | 2.1E−09 |
| VL1.M2-VH2.M6 | Humanized | 1.9E+05 | 5.0E−04 | 2.7E−09 |

### Example 2: Generation and Screening of Humanized Anti-GPC3 CAR T Cells In Vitro Generation of CAR Constructs

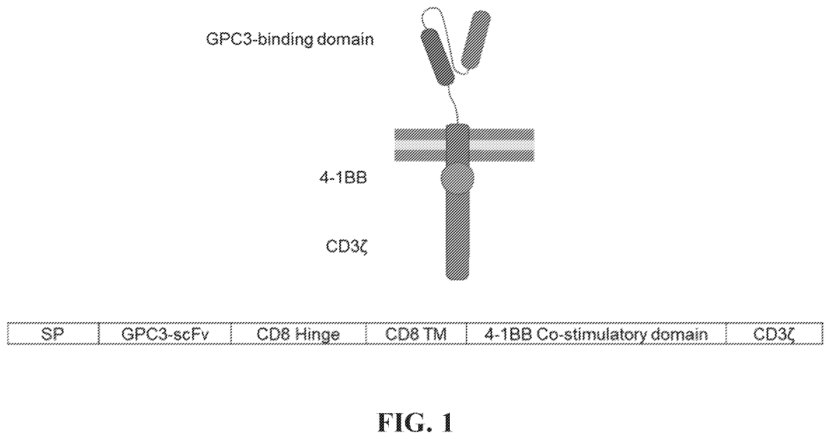

Each isolated optimized binder (SEQ ID NOs: 39-46) was cloned into a lentiviral expression vector with the intracellular co-stimulatory sequence of 4-1BB and intracellular domain of CD3ξ as shown in FIG. 1. The CAR constructs (Table 2, SEQ ID NOs: 54-61) were cloned into-an expression vector with an EF1α promoter for expression.

TABLE 2
Exemplary anti-GPC3 chimeric receptors

| | VH | VH2 | VH2.M1 | VH2.M5 | VH2.M6 |
|---|---|---|---|---|---|
| VL | / | / | / | / | / |
| VL1 | / | construct185 | construct186 | construct187 | construct188 |
| VL1.M2 | / | construct189 | construct190 | construct191 | construct192 |

### Preparation of Lentivirus

The lentivirus packaging plasmid mixture including pCMV-ΔR-8.47 and pMD2.G (Addgene, Cat #12259) was pre-mixed with each vector PLLV-hEF 1a-GPC3 comprising a CAR construct at a pre-optimized ratio with polyethylenimine. The mixture was then added to the HEK293 cells. Supernatants from the cells were collected after overnight incubation. The virus-containing supernatants were filtered through a 0.45 μm PES filter, followed by ultra-centrifugation to pellet the lentivirus. The virus pellets were rinsed with pre-chilled PBS. The virus was aliquoted and stored at −80° C. immediately. The virus titer was determined by measurement of transduction efficiency to supT1 cell line using a flow cytometry assay.

### Collection and Transduction of αβ T Lymphocytes

Leukocytes were collected from healthy donors by apheresis. Peripheral blood mononuclear cells (PBMCs) were isolated using FICOLL-PAQUE™ PLUS Media (GE Healthcare, Cat #17-5442-02) according to manufacturer's protocol. Human T cells were purified from PMBCs using a Pan-T cell isolation kit (Miltenyi, Cat #130-096-535), following manufacturer's protocol. The purified T cells were subsequently pre-activated for 48 hours with a human T cell activation/expansion kit (Miltenyi, Cat #130-091-441) according to manufacturer's protocol in which anti-CD3/CD28 MACSiBead particles were added at a bead-to-cell ratio of 1:2. The pre-activated T cells were transduced with each lentivirus stock in the presence of 7 μg/ml polybrene. The transduced cells were then transferred to the cell culture incubator for transgene expression under suitable conditions. Cells are harvested 6 days post-transduction, the total number and transduction efficiency are determined.

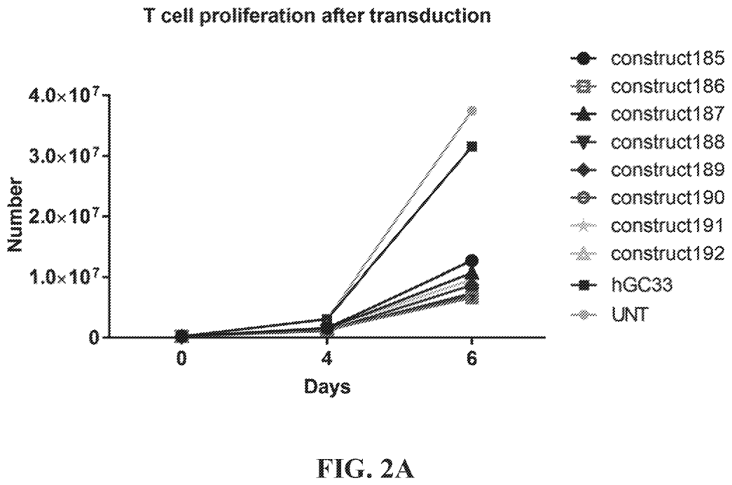

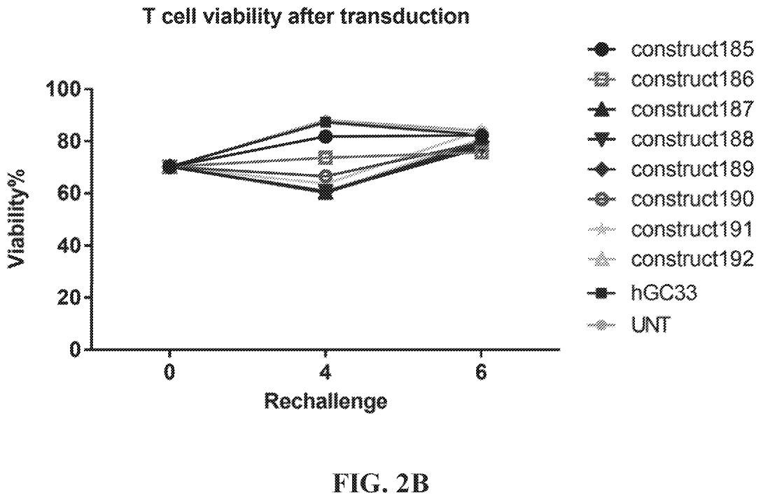

For anti-GPC3 CAR-T staining, cells are stained with PE anti-DYKDDDDK Tag Antibody (Biolegend). Flow cytometry analysis for all experiments is performed by using FlowJo (Tree Star, Inc.). The positive rates of virus infected T cells expressing different CARs are shown in Table 3. Among them, a CAR with anti-GPC3 antibody hGC33 VH and VL sequences was used for comparison purpose. The hGC33 antibody was described e.g., Ishiguro, et al., Cancer research 68.23 (2008): 9832-9838, which is incorporated herein by reference in its entirety. The viability and expansion of anti-GPC3 CAR αβ T cells are shown in FIG. 2A-2B.

TABLE 3
Transfection efficiency of exemplary anti-GPC3 CAR αβ T cells

| CAR | Positive rate of T cells transfection (%) |
|---|---|
| construct185 | 42.3 |
| construct186 | 40.8 |
| construct187 | 49.5 |
| construct188 | 57.1 |
| construct189 | 55.7 |
| construct190 | 52.1 |
| construct191 | 52.6 |
| construct192 | 38.2 |
| hGC33 | 19.0 |

### Long-Term Cytotoxicity Assay of Anti-GPC3 CAR αβ T Cells

To evaluate the long-term killing efficacy of CAR αβ T cells, we performed long-term co-culture assays, which mimic the dynamic killing process in vivo. Transduced or non-transduced T cells (1×10⁵/well) were co-cultured with tumor cell lines (Huh7 cells, 1×10⁵/well) at an E:T ratio of 1:1 in 24-well plates, in the absence of exogenous cytokines (IL-2). Part of the cells were harvested and stained for CD3 after 2 or 3 days' co-culture. For serial co-culture assays, the remaining T cells were then re-challenged with fresh Huh7 cells at the same E:T ratio. Co-cultures were carried on until tumor cells outgrew. The T cell proliferation rate at each time point is calculated by dividing the number of T cells at the time point by the number of T cells at the initial time point.

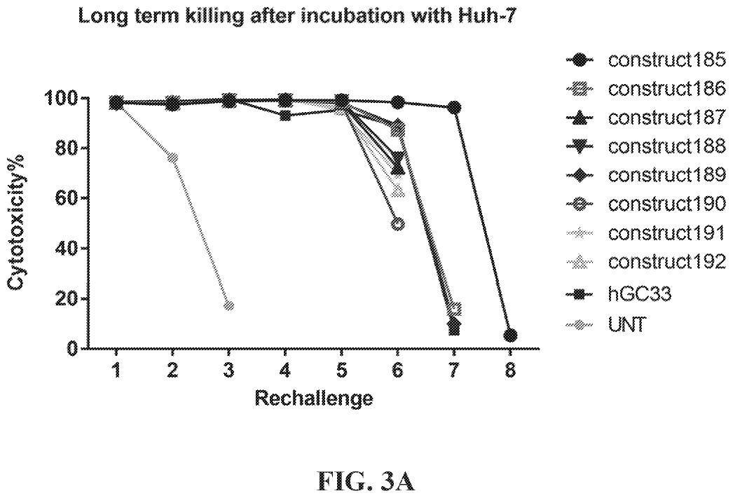

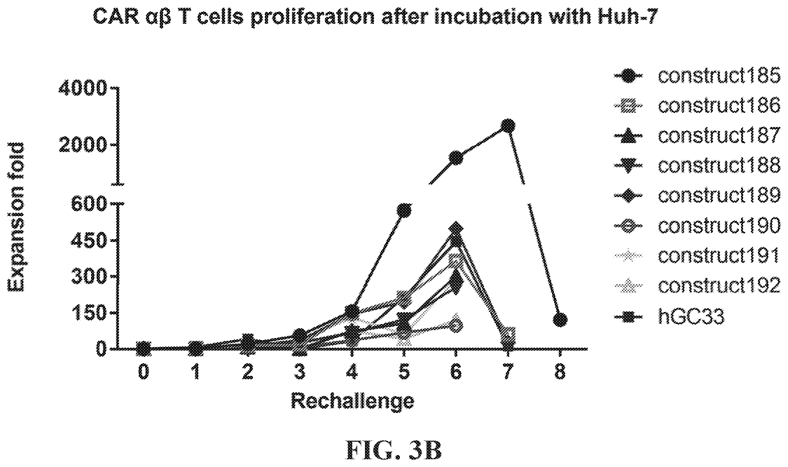

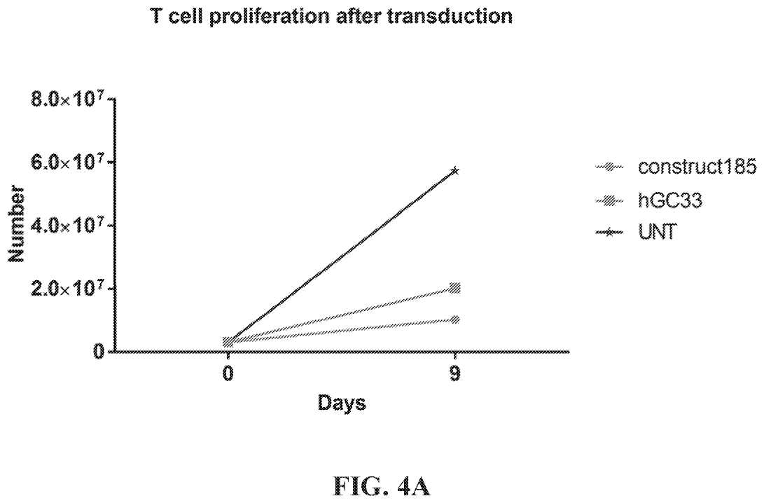

Representative result of long-term co-culture assay by FACS detection was shown in FIG. 3A. Calculated T cell proliferation from the same experiment were shown in FIG. 3B. The data indicated that construct 185 CAR αβ T cells had better cytotoxicity and proliferation than hGC33 CAR αβ T cells, and the efficacy of construct 186 and 189 CAR αβ T cells were comparable to hGC33 CAR αβ T cells in vitro.

### Collection and Transduction of γδ T Lymphocytes

T6 T cells were prepared by addition of 5 μM Zoledronate and 1000 IU/mL IL-2 to PBMCs and cultured for 9 days with periodical change of media supplemented with 1000 IU/mL IL-2. Alternatively, γδ T cells were isolated from PBMC or umbilical cord blood (UCB) and then stimulated by anti-γδ TCR antibody and anti-CD3 (OKT3) followed by co-incubation of K562-based artificial antigen-presenting cells (aAPCs) at an 1:2 ratio for at least 10 days.

PBMCs were isolated by density centrifugation (lymphoprep) from leukapheresis material and cryopreserved. PBMCs were resuscitated and activated with zoledronic acid (5M) in cell culture media AIMN-V supplemented with IL-2 (1000 IU/ml) and 5% human AB serum and kept in a humidified chamber (37° C., 5% C02). 48 hours post-activation, cells were transduced with lentiviral vectors encoding the system of Example 1 at an MOI of 5 with 5 μg/ml polybrene. Such transduction procedure was repeated the next day followed by replenishment of fresh media containing IL-2 (1000 IU/ml) the second day after the transduction. Cells were cultured in AIM-V supplemented with IL-2 (1000 IU/ml) in a humidified chamber with periodical change of media as determined by the pH of the culture media for further expansion. Cells were harvested 10 days post-transduction and the total number, purity and transduction efficiency were determined. Cells were further enriched with a negative TCRγ/δ+ T cell isolation kit (Miltenyi Biotec) before future applications or cryopreserved.

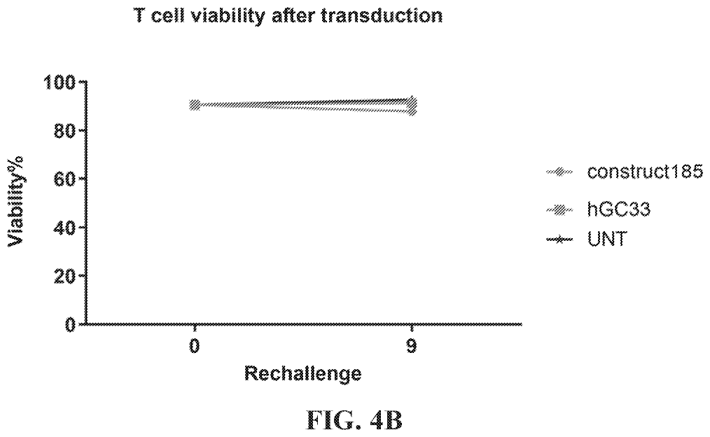

The positive rates of virus infected T cells expressing different CARs are shown in Table 4. The viability and expansion of anti-GPC3 CAR γδ T cells are shown in FIG. 4A-4B.

TABLE 4
Transfection efficiency of exemplary anti-GPC3 CAR γδ T cells

| CAR | Positive rate of T cells transfection (%) |
|---|---|
| construct185 | 60.9 |
| hGC33 | 53.2 |

### Long-Term Cytotoxicity Assay of Anti-GPC3 CAR γδ T Cells

The long-term killing efficacy of CAR γδ T cells against tumor cells was assessed using long-term co-culture assays as described in CAR αβ T cells.

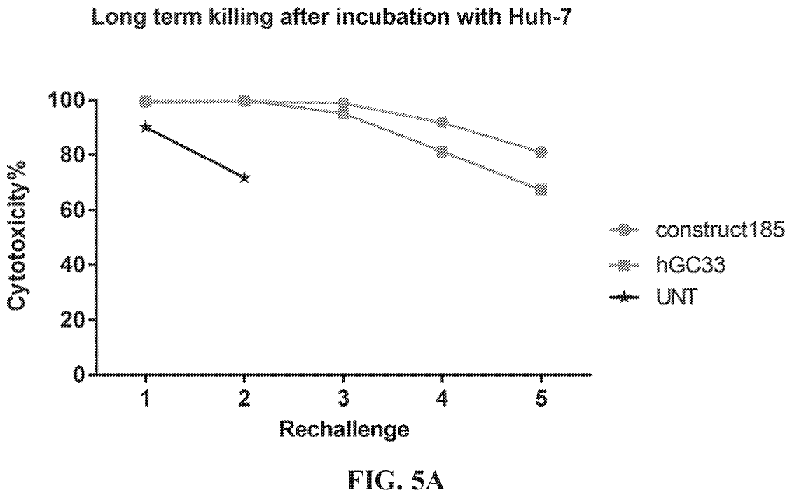

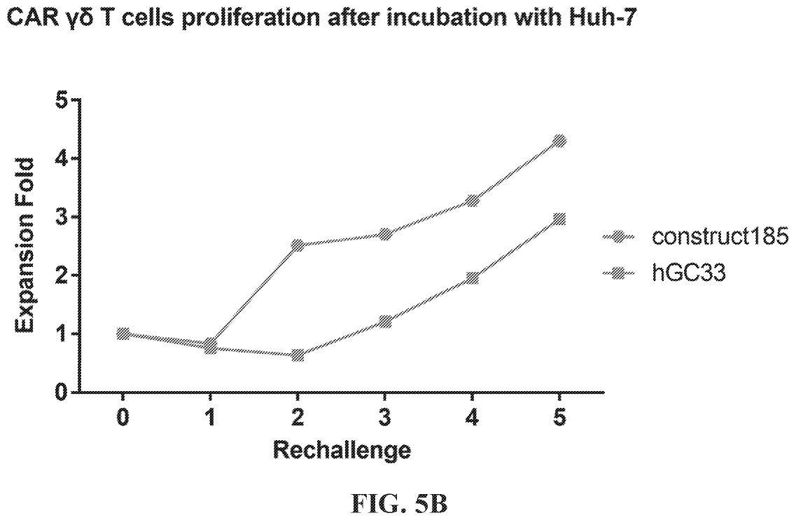

Representative result of long-term co-culture assay by FACS detection was shown in FIG. 5A. Calculated T cell proliferation from the same experiment were shown in FIG. 5B. The data indicated that the cytotoxicity and proliferation of construct185 CAR γδ T cells were better than hGC33 CAR γδ T cells in vitro.

### Example 3: In Vivo Efficacy Evaluation of Anti-GPC3 CAR γδT Cells in Mouse Xenograft Model

Anti-tumor activity of an exemplary anti-GPC3 CAR γδ T cells was assessed in vivo in a Huh7 xenograft model. Briefly, 3 million (3×10⁶) huh7 cells were implanted subcutaneously on day 0 in NOD/SCID IL-2RγC null (NSG) mice. Ten days after tumor inoculation, mice were treated with intravenous injection of 1×10⁶ CAR-T or mock T cells or phosphate-buffered saline (PBS). Tumor dimensions were measured with calipers twice a week, and tumor volumes were calculated using the formula V=1/2 (length×width²). Mice were euthanized when the mean tumor burden in the control mice reached 2,000 mm³.

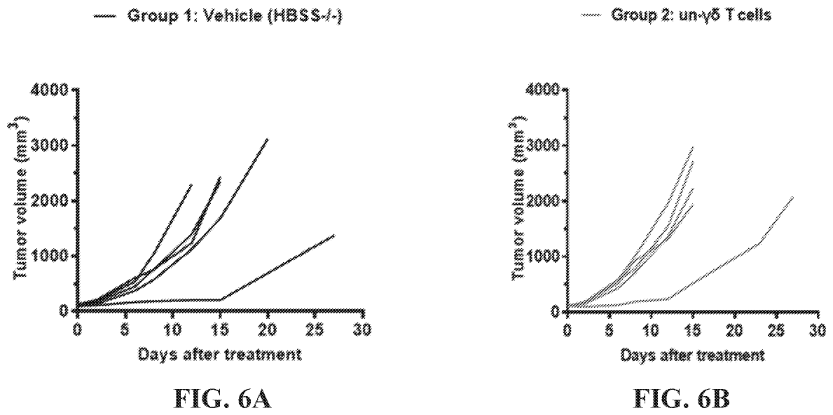

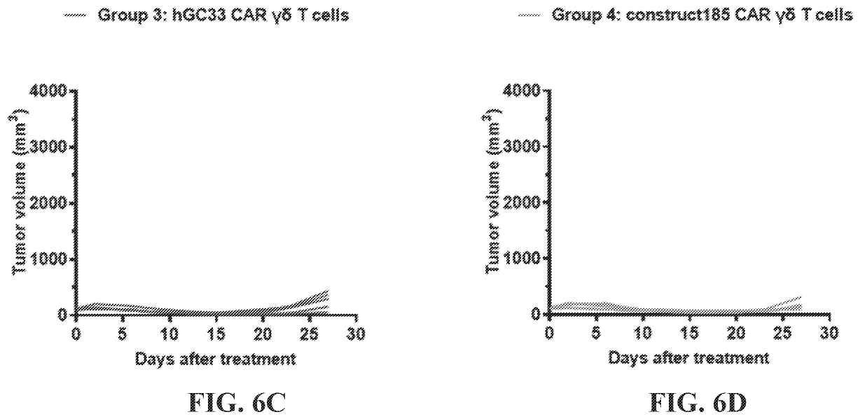

As shown in FIGS. 6A-6D, mice treated with UnT or vehicle exhibited rapid tumor progression and had to be euthanized before the end of the experiment, while mice treated with anti-GPC3 CAR γδ T cells were tumor free after 2-3 weeks post infusion, meanwhile construct 185 CAR γδ T cells had better efficacy than hGC33 CAR γδ T cells in vivo.

### Example 4: Further Humanization Improvement of Humanized Anti-GPC3 scFvs

To further improve the humanization of anti-GPC3 scFvs, additional amino acid residues in VH1 and VL1 were mutated to their human germline counterparts. In HCDR2 of VH2.M6, mutations K54S, T55G or both were introduced, yielding VH2.M8 (SEQ ID NO: 11), VH2.M9 (SEQ ID NO: 12) and VH2.M10 (SEQ ID NO: 13) sequences, respectively. Mutation D57S was introduced to VH2.M6, yielding VH2.M11 sequence (SEQ ID NO: 14). All three additional mutations was introduced to VH2.M6, yielding VH2.M12 sequence (SEQ ID NO: 15). Similarly for VL1.M2, an additional N35K mutation was introduced to LCDR1, yielding VL1.M3 (SEQ ID NO: 16). Additional S94M, N96G and V99W mutations were introduced to LCDR3 of VL1.M2 individually, yielding VL1.M6 (SEQ ID NO: 17), VL1.M7 (SEQ ID NO: 18) and VL1.M8 (SEQ ID NO: 19) sequences, respectively. Additional S94M mutations was introduced to LCDR3 of VL1, yielding VL1.M9 (SEQ ID NO: 137) sequence. The binding affinity of these humanized variants were again determined using SPR as described in Example 1 (Table 5). According to the result, further mutations in HCDR2 did not affect the binding affinity of VH2.M6 significantly. The VH variant with the lowest binding affinity is VH2.M11 the affinity of which dropped less than 4-fold. On the other hand, further mutations in VL1M2 CDRs did affect its binding affinity, especially LCDR3 mutations, except the S94M mutation which has a binding affinity which is nearly the same as that of VL1.M2. CDR sequences of humanized VHs and VLs are showed in Table 6 and Table 7, respectively.

TABLE 5
Binding affinity of mouse and further humanized scFvs.

| | Species | kₐ (1/Ms) | k_{d} (1/s) | K_{D} (M) |
|---|---|---|---|---|
| VL-VH | Mouse | 5.1E+04 | 2.1E−04 | 4.0E−09 |
| VL1M2-VH2M6 | Humanized | 7.0E+04 | 4.8E−04 | 6.8E−09 |
| VL1M2-VH2M8 | Humanized | 7.4E+04 | 4.4E−04 | 6.0E−09 |
| VL1M2-VH2M9 | Humanized | 7.4E+04 | 6.0E−04 | 8.2E−09 |
| VL1M2-VH2M10 | Humanized | 7.4E+04 | 5.0E−04 | 6.8E−09 |
| VL1M2-VH2M11 | Humanized | 6.7E+04 | 8.5E−04 | 1.3E−08 |
| VL1M3-VH2M6 | Humanized | 6.1E+04 | 1.6E−03 | 2.7E−08 |
| VL1M6-VH2M6 | Humanized | 1.0E+05 | 5.8E−04 | 5.7E−09 |
| VL1M7-VH2M6 | Humanized | 6.7E+04 | 3.4E−03 | 5.1E−08 |
| VL1M8-VH2M6 | Humanized | 9.4E+04 | 4.2E−03 | 4.4E−08 |

TABLE 6
CDR sequences of humanized VHs

| | HCDR1 | | HCDR2 | | HCDR3 | |
|---|---|---|---|---|---|---|
| | SEQ | Se- | SEQ | Se- | SEQ | Se- |
| VH | ID | quence | ID | quence | ID | quence |
| Mouse | 20 | GYTFT | 22 | ALDPKTG | 29 | FYSYTY |
| | | DYEMH | | DTAYSQK | | |
| | | | | FKG | | |
| VH1 | 20 | GYTFT | 22 | ALDPKTG | 29 | FYSYTY |
| | | DYEMH | | DTAYSQK | | |
| | | | | FKG | | |
| VH2 | 20 | GYTFT | 22 | ALDPKTG | 29 | FYSYTY |
| | | DYEMH | | DTAYSQK | | |
| | | | | FKG | | |
| VH2.M1 | 21 | GYTFT | 22 | ALDPKTG | 29 | FYSYTY |
| | | SYEMH | | DTAYSQK | | |
| | | | | FKG | | |
| VH2.M5 | 20 | GYTFT | 23 | ALDPKTG | 29 | FYSYTY |
| | | DYEMH | | DTAYSQK | | |
| | | | | FQG | | |
| VH2.M6 | 21 | GYTFT | 23 | ALDPKTG | 29 | FYSYTY |
| | | SYEMH | | DTAYSQK | | |
| | | | | FQG | | |
| VH2.M8 | 21 | GYTFT | 24 | ALDPSTG | 29 | FYSYTY |
| | | SYEMH | | DTAYSQK | | |
| | | | | FQG | | |
| VH2.M9 | 21 | GYTFT | 25 | ALDPKGG | 29 | FYSYTY |
| | | SYEMH | | DTAYSQK | | |
| | | | | FQG | | |
| VH2.M10 | 21 | GYTFT | 26 | ALDPSGG | 29 | FYSYTY |
| | | SYEMH | | DTAYSQK | | |
| | | | | FQG | | |
| VH2.M11 | 21 | GYTFT | 27 | ALDPKTG | 29 | FYSYTY |
| | | SYEMH | | STAYSQK | | |
| | | | | FQG | | |
| VH2.M12 | 21 | GYTFT | 28 | ALDPSGG | 29 | FYSYTY |
| | | SYEMH | | STAYSQK | | |
| | | | | FQG | | |

TABLE 7
CDR sequences of humanized VLs

| | LCDR1 | | LCDR2 | | LCDR3 | |
|---|---|---|---|---|---|---|
| | SEQ | Se- | SEQ | Se- | SEQ | Se- |
| VL | ID | quence | ID | quence | ID | quence |
| Mouse | 30 | RSSQSLV | 32 | KVSNR | 34 | SQNTH |
| | | HSNGNTY | | FS | | VPPT |
| | | LH | | | | |
| VL1 | 30 | RSSQSLV | 32 | KVSNR | 34 | SQNTH |
| | | HSNGNTY | | FS | | VPPT |
| | | LH | | | | |
| VL1.M2 | 30 | RSSQSLV | 33 | KVSSR | 34 | SQNTH |
| | | HSNGNTY | | FS | | VPPT |
| | | LH | | | | |
| VL1.M3 | 31 | RSSQSLV | 33 | KVSSR | 34 | SQNTH |
| | | HSNGKTY | | FS | | VPPT |
| | | LH | | | | |
| VL1.M6 | 30 | RSSQSLV | 33 | KVSSR | 35 | MQNTH |
| | | HSNGNTY | | FS | | VPPT |
| | | LH | | | | |
| VL1.M7 | 30 | RSSQSLV | 33 | KVSSR | 36 | SQGTH |
| | | HSNGNTY | | FS | | VPPT |
| | | LH | | | | |
| VL1.M8 | 30 | RSSQSLV | 33 | KVSSR | 37 | SQNTH |
| | | HSNGNTY | | FS | | WPPT |
| | | LH | | | | |
| VL1.M9 | 30 | RSSQSLV | 32 | KVSNR | 35 | MQNTH |
| | | HSNGNTY | | FS | | VPPT |
| | | LH | | | | |

### Example 5: Generation and Screening of Further Humanized Anti-GPC3 CAR T Cells In Vitro Generation of CAR Constructs

Each isolated further humanized binder (SEQ TD NOs: 62-96) was cloned into a lentiviral expression vector with the intracellular co-stimulatory sequence of 4-1BB1 and intracellular domain of CD3C as shown in FIG. 1. The CAR constructs (Table 8, SEQ ID NOs: 97-131) were cloned into an expression vector with an EF1α promoter for expression.

TABLE 8
Exemplary anti-GPC3 chimeric receptors

| | VH2 | VH2.M8 | VH2.M9 | VH2.M10 | VH2.M11 | VH2.M12 |
|---|---|---|---|---|---|---|
| VL1 | construct185 | construct195 | construct196 | construct197 | construct198 | construct199 |
| VL1.M3 | construct200 | construct201 | construct202 | construct203 | construct204 | construct205 |
| VL1.M6 | construct206 | construct207 | construct208 | construct209 | construct210 | construct211 |
| VL1.M7 | construct212 | construct213 | construct214 | construct215 | construct216 | construct217 |
| VL1.M8 | construct218 | construct219 | construct220 | construct221 | construct222 | construct223 |
| VL1.M9 | construct224 | construct225 | construct226 | construct227 | construct228 | construct229 |

### Preparation of Lentivirus

Lentiviruses encoding further humanized CARs (construct95-construct229) were prepared as described in Example 2.

### Collection and Transduction of αβ T Lymphocytes

αβ T lymphocytes were collected and transduced with the lentiviruses according to the protocol in Example 2. The positive rates of virus infected T cells expressing different CARs were shown in Table 9-11.

TABLE 9
Transfection efficiency of exemplary anti-GPC3 CAR αβ T cells

| CAR | Positive rate of T cells transfection (%) |
|---|---|
| construct195 | 40.0 |
| construct196 | 32.4 |
| construct197 | 30.3 |
| construct198 | 46.5 |
| construct199 | 8.60 |
| construct200 | 40.6 |
| construct201 | 21.8 |
| construct202 | 10.9 |
| construct203 | 28.4 |
| construct204 | 36.4 |
| construct205 | 30.0 |
| hGC33 | 29.6 |

TABLE 10
Transfection efficiency of exemplary anti-GPC3 CAR αβ T cells

| CAR | Positive rate of T cells transfection (%) |
|---|---|
| Construct206 | 53.6 |
| construct207 | 40.3 |
| construct208 | 17.7 |
| construct209 | 25.1 |
| construct210 | 49.8 |
| construct211 | 37.7 |
| construct212 | 42.2 |
| construct213 | 34.6 |
| construct214 | 16.1 |
| construct215 | 7.96 |
| construct216 | 30.8 |
| construct217 | 11.6 |
| hGC33 | 36.6 |

TABLE 11
Transfection efficiency of exemplary anti-GPC3 CAR αβ T cells

| CAR | Positive rate of T cells transfection (%) |
|---|---|
| construct218 | 0.06 |
| construct219 | 23.4 |
| construct220 | 15.7 |
| construct221 | 9.10 |
| construct222 | 30.8 |
| construct223 | 15.6 |
| construct224 | 33.6 |
| construct225 | 23.4 |
| construct226 | 19.0 |
| construct227 | 19.0 |
| construct228 | 25.2 |
| construct229 | 2.62 |
| hGC33 | 23.5 |

### Long-Term Cytotoxicity Assay of Anti-GPC3 CAR αβ T Cells

The long-term killing efficacy of CAR αβ 3 T cells against tumor cells was assessed using long-term co-culture assays as described in Example 2.

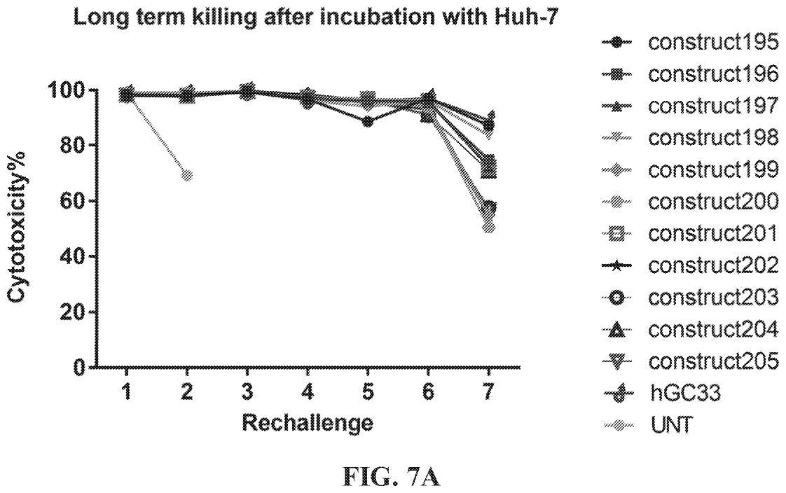

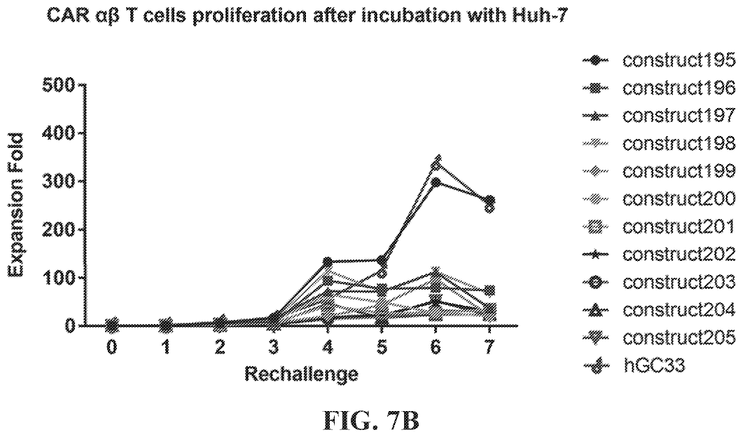

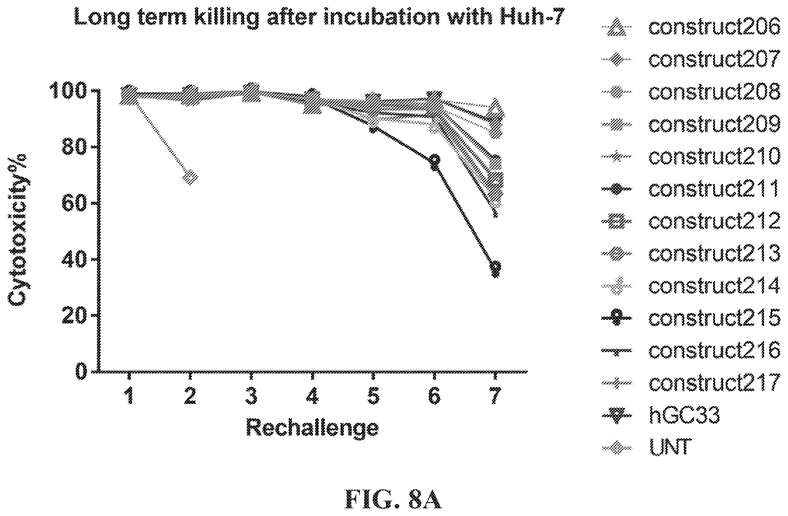

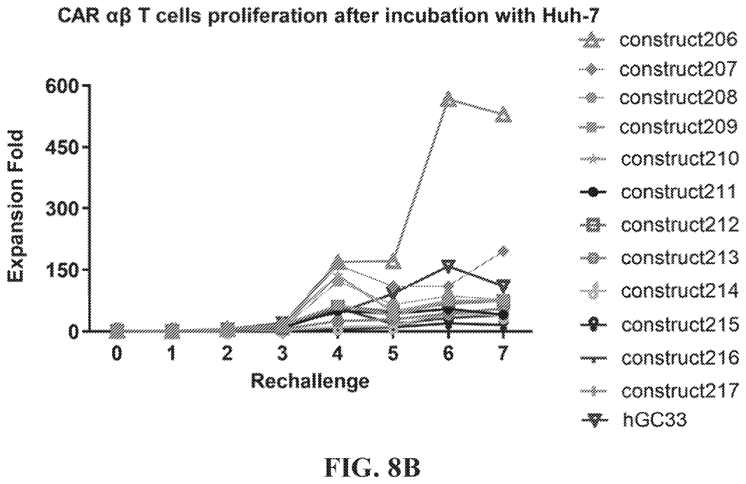

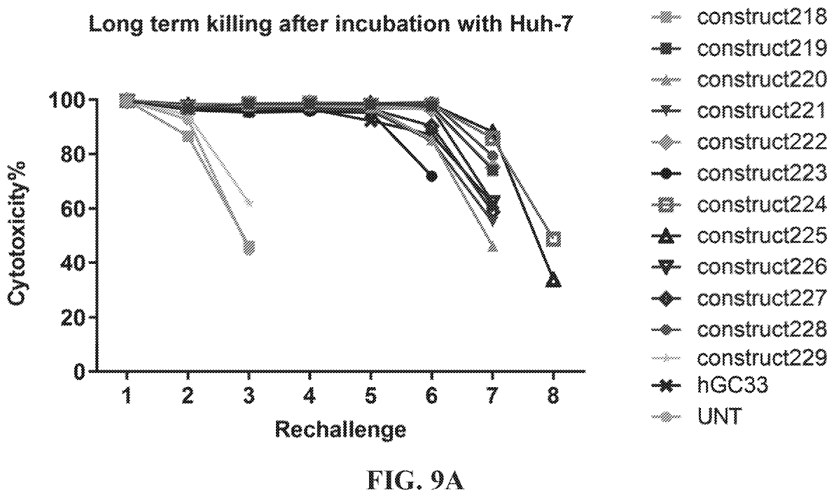

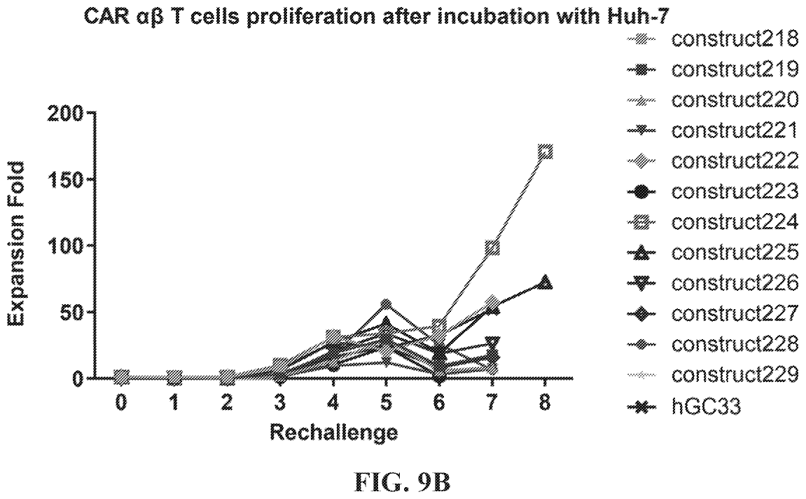

Representative results of long-term co-culture assay by FACS detection were shown in FIGS. 7A, 8A and 9A. Calculated T cell proliferation from the same experiment were shown in FIGS. 71B, 8B and 9B. The data indicated that further humanized construct206, 224 and 225 CAR αβ 3 T cells have better cytotoxicity and proliferation than hGC33 CAR αβ 3 T cells, and the efficacy of construct 195, 207, 219, 222, 226 and 228 CAR αβ 3 T cells were comparable to hGC33 CAR αβ 3 T cells in vitro.

### Collection and Transduction of γδ T Lymphocytes

γδ T lymphocytes were collected and transduced with the lentiviruses according to the protocol in Example 2. The positive rates of virus infected T cells expressing different CARs were shown in Table 12.

TABLE 12
Transfection efficiency of exemplary anti-GPC3 CAR γδ T cells

| CAR | Positive rate of T cells transfection (%) |
|---|---|
| construct185 | 52.7 |
| construct206 | 41.2 |
| construct224 | 50.7 |
| construct225 | 42.2 |
| hGC33 | 55.8 |

### Long-Term Cytotoxicity Assay of Anti-GPC3 CAR γδ T Cells

The long-term killing efficacy of CAR γδ T cells against tumor cells is assessed using long-term co-culture assays as described in Example 2.

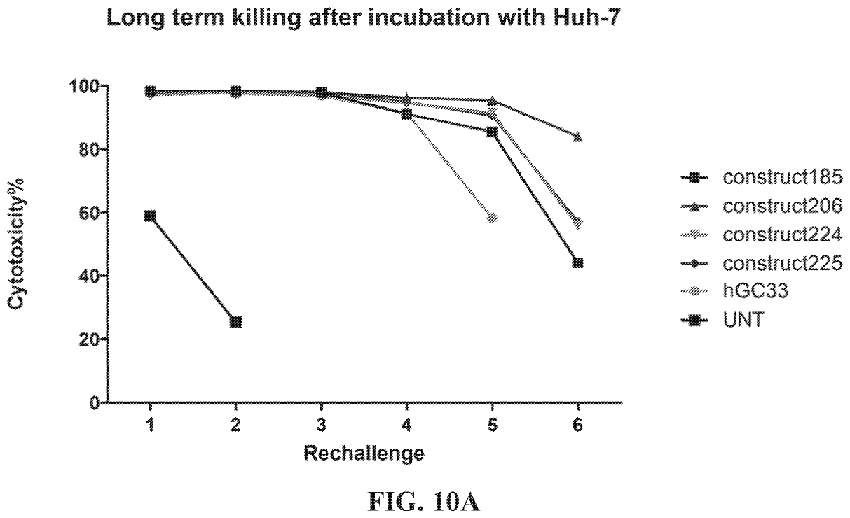

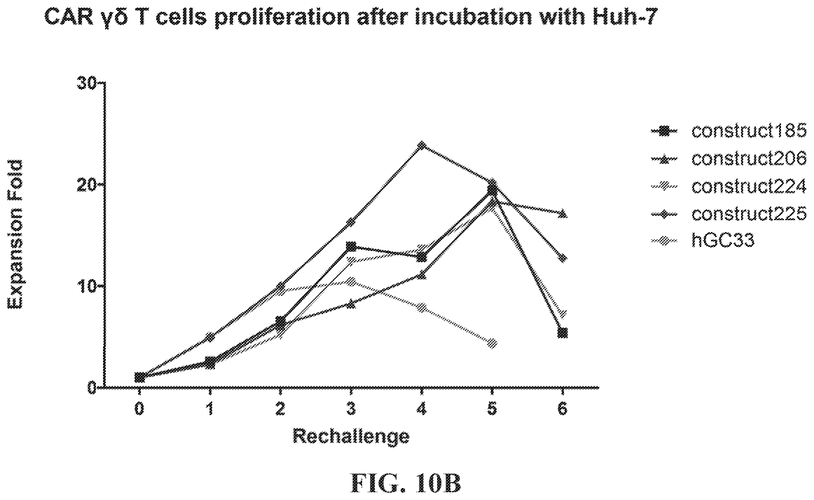

Representative result of long-term co-culture assay by FACS detection was shown in FIG. 10A. Calculated T cell proliferation from the same experiment were shown in FIG. 10B. The data indicated that the cytotoxicity and proliferation of construct185, 206, 224 and 225 CAR T6 T cells were better than hGC33 CAR γδ T cells in vitro.

### Example 6: In Vivo Efficacy Evaluation of Anti-GPC3 CAR γδT Cells in Mouse Xenograft Model

Anti-tumor activity of anti-GPC3 CAR γδ T cells is assessed in a Huh7 xenograft model as described in Example 3.

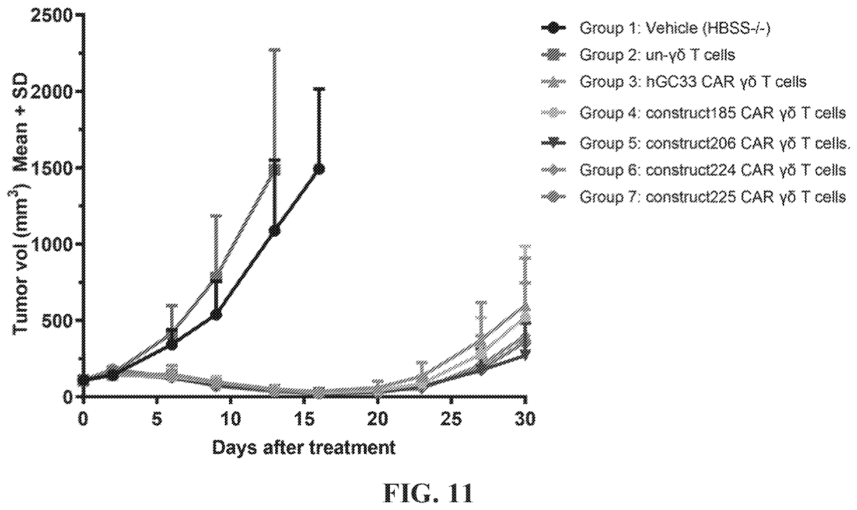

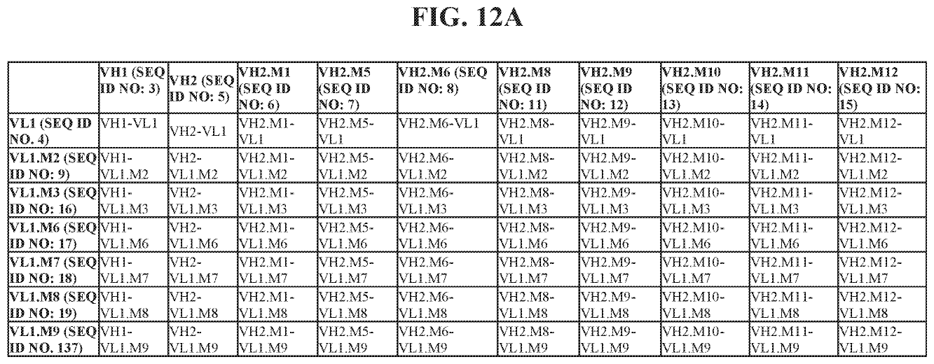

As shown in FIG. 11, mice treated with UnT or vehicle exhibited rapid tumor progression and had to be euthanized before the end of the experiment, while mice treated with anti-GPC3 CAR γδ T cells were tumor free after 2-3 weeks post infusion, meanwhile further humanized construct206, 224 and 225 CAR γδ T cells have better efficacy than hGC33 CAR γδ T cells in vivo.

### Other Embodiments

It is to be understood that while the disclosure has been described in conjunction with the detailed description thereof, the foregoing description is intended to illustrate and not limit the scope of the disclosure, which is defined by the scope of the appended claims. Other aspects, advantages, and modifications are within the scope of the following claims.

This listing of claims replaces all prior versions and listings of claims in the application:

1. An antibody or antigen-binding fragment thereof that binds to GPC3, comprising:
a heavy chain variable region (VH) comprising VH complementarity determining regions (CDRs) 1, 2, and 3; and
a light chain variable region (VL) comprising VL CDRs 1, 2, and 3,
wherein the VH CDRs 1, 2, and 3 amino acid sequences and the VL CDRs, 1, 2, and 3 amino acid sequences are one of the following:
(1) the VH CDRs 1, 2, 3 amino acid sequences are set forth in SEQ ID NOs: 20, 22, and 29 respectively, and the selected VL CDRs 1, 2, 3 amino acid sequences are set forth in SEQ ID NOs: 30, 32, and 34 respectively;
(2) the VH CDRs 1, 2, 3 amino acid sequences are set forth in SEQ ID NOs: 21, 22, and 29 respectively, and the selected VL CDRs 1, 2, 3 amino acid sequences are set forth in SEQ ID NOs: 30, 32 and 34 respectively;
(3) the VH CDRs 1, 2, 3 amino acid sequences are set forth in SEQ ID NOs: 20, 23 and 29 respectively, and the selected VL CDRs 1, 2, 3 amino acid sequences are set forth in SEQ ID NOs: 30, 32 and 34 respectively;
(4) the VH CDRs 1, 2, 3 amino acid sequences are set forth in SEQ ID NOs: 21, 23, and 29 respectively, and the selected VL CDRs 1, 2, 3 amino acid sequences are set forth in SEQ ID NOs: 30, 32 and 34 respectively;
(5) the VH CDRs 1, 2, 3 amino acid sequences are set forth in SEQ ID NOs: 20, 22 and 29 respectively, and the selected VL CDRs 1, 2, 3 amino acid sequences are set forth in SEQ ID NOs: 30, 33 and 34 respectively;
(6) the VH CDRs 1, 2, 3 amino acid sequences are set forth in SEQ ID NOs: 21, 22, and 29 respectively, and the selected VL CDRs 1, 2, 3 amino acid sequences are set forth in SEQ ID NOs: 30, 33 and 34 respectively;
(7) the VH CDRs 1, 2, 3 amino acid sequences are set forth in SEQ ID NOs: 20, 23, and 29 respectively, and the selected VL CDRs 1, 2, 3 amino acid sequences are set forth in SEQ ID NOs: 30, 33 and 34 respectively;
(8) the VH CDRs 1, 2, 3 amino acid sequences are set forth in SEQ ID NOs: 21, 23, and 29 respectively, and the selected VL CDRs 1, 2, 3 amino acid sequences are set forth in SEQ ID NOs: 30, 33 and 34 respectively;
(9) the VH CDRs 1, 2, 3 amino acid sequences are set forth in SEQ ID NOs: 21, 24, and 29 respectively, and the selected VL CDRs 1, 2, 3 amino acid sequences are set forth in SEQ ID NOs: 30, 33 and 34 respectively;
(10) the VH CDRs 1, 2, 3 amino acid sequences are set forth in SEQ ID NOs: 21, 25, and 29 respectively, and the selected VL CDRs 1, 2, 3 amino acid sequences are set forth in SEQ ID NOs: 30, 33 and 34 respectively;
(11) the VH CDRs 1, 2, 3 amino acid sequences are set forth in SEQ ID NOs: 21, 26, and 29 respectively, and the selected VL CDRs 1, 2, 3 amino acid sequences are set forth in SEQ ID NOs: 30, 33 and 34 respectively;
(12) the VH CDRs 1, 2, 3 amino acid sequences are set forth in SEQ ID NOs: 21, 27, and 29 respectively, and the selected VL CDRs 1, 2, 3 amino acid sequences are set forth in SEQ ID NOs: 30, 33 and 34 respectively;
(13) the VH CDRs 1, 2, 3 amino acid sequences are set forth in SEQ ID NOs: 21, 23 and 29 respectively, and the selected VL CDRs 1, 2, 3 amino acid sequences are set forth in SEQ ID NOs: 31, 33 and 34 respectively;
(14) the VH CDRs 1, 2, 3 amino acid sequences are set forth in SEQ ID NOs: 21, 23 and 29 respectively, and the selected VL CDRs 1, 2, 3 amino acid sequences are set forth in SEQ ID NOs: 30, 33 and 35 respectively;
(15) the VH CDRs 1, 2, 3 amino acid sequences are set forth in SEQ ID NOs: 21, 23 and 29 respectively, and the selected VL CDRs 1, 2, 3 amino acid sequences are set forth in SEQ ID NOs: 30, 33 and 36 respectively;
(16) the VH CDRs 1, 2, 3 amino acid sequences are set forth in SEQ ID NOs: 21, 23 and 29 respectively, and the selected VL CDRs 1, 2, 3 amino acid sequences are set forth in SEQ ID NOs: 30, 33 and 37 respectively;
(17) the VH CDRs 1, 2, 3 amino acid sequences are set forth in SEQ ID NOs: 21, 24 and 29 respectively, and the selected VL CDRs 1, 2, 3 amino acid sequences are set forth in SEQ ID NOs: 30, 32 and 34 respectively;
(18) the VH CDRs 1, 2, 3 amino acid sequences are set forth in SEQ ID NOs: 21, 25 and 29 respectively, and the selected VL CDRs 1, 2, 3 amino acid sequences are set forth in SEQ ID NOs: 30, 32 and 34 respectively;
(19) the VH CDRs 1, 2, 3 amino acid sequences are set forth in SEQ ID NOs: 21, 26 and 29 respectively, and the selected VL CDRs 1, 2, 3 amino acid sequences are set forth in SEQ ID NOs: 30, 32 and 34 respectively;
(20) the VH CDRs 1, 2, 3 amino acid sequences are set forth in SEQ ID NOs: 21, 27 and 29 respectively, and the selected VL CDRs 1, 2, 3 amino acid sequences are set forth in SEQ ID NOs: 30, 32 and 34 respectively;
(21) the VH CDRs 1, 2, 3 amino acid sequences are set forth in SEQ ID NOs: 21, 28 and 29 respectively, and the selected VL CDRs 1, 2, 3 amino acid sequences are set forth in SEQ ID NOs: 30, 32 and 34 respectively;
(22) the VH CDRs 1, 2, 3 amino acid sequences are set forth in SEQ ID NOs: 20, 22 and 29 respectively, and the selected VL CDRs 1, 2, 3 amino acid sequences are set forth in SEQ ID NOs: 31, 33 and 34 respectively;
(23) the VH CDRs 1, 2, 3 amino acid sequences are set forth in SEQ ID NOs: 21, 24 and 29 respectively, and the selected VL CDRs 1, 2, 3 amino acid sequences are set forth in SEQ ID NOs: 31, 33 and 34 respectively;
(24) the VH CDRs 1, 2, 3 amino acid sequences are set forth in SEQ ID NOs: 21, 25 and 29 respectively, and the selected VL CDRs 1, 2, 3 amino acid sequences are set forth in SEQ ID NOs: 31, 33 and 34 respectively;
(25) the VH CDRs 1, 2, 3 amino acid sequences are set forth in SEQ ID NOs: 21, 26 and 29 respectively, and the selected VL CDRs 1, 2, 3 amino acid sequences are set forth in SEQ ID NOs: 31, 33 and 34 respectively;
(26) the VH CDRs 1, 2, 3 amino acid sequences are set forth in SEQ ID NOs: 21, 27 and 29 respectively, and the selected VL CDRs 1, 2, 3 amino acid sequences are set forth in SEQ ID NOs: 31, 33 and 34 respectively;
(27) the VH CDRs 1, 2, 3 amino acid sequences are set forth in SEQ ID NOs: 21, 28 and 29 respectively, and the selected VL CDRs 1, 2, 3 amino acid sequences are set forth in SEQ ID NOs: 31, 33 and 34 respectively;
(28) the VH CDRs 1, 2, 3 amino acid sequences are set forth in SEQ ID NOs: 20, 22 and 29 respectively, and the selected VL CDRs 1, 2, 3 amino acid sequences are set forth in SEQ ID NOs: 30, 33 and 35 respectively;
(29) the VH CDRs 1, 2, 3 amino acid sequences are set forth in SEQ ID NOs: 21, 24 and 29 respectively, and the selected VL CDRs 1, 2, 3 amino acid sequences are set forth in SEQ ID NOs: 30, 33 and 35 respectively;
(30) the VH CDRs 1, 2, 3 amino acid sequences are set forth in SEQ ID NOs: 21, 25 and 29 respectively, and the selected VL CDRs 1, 2, 3 amino acid sequences are set forth in SEQ ID NOs: 30, 33 and 35 respectively;
(31) the VH CDRs 1, 2, 3 amino acid sequences are set forth in SEQ ID NOs: 21, 26 and 29 respectively, and the selected VL CDRs 1, 2, 3 amino acid sequences are set forth in SEQ ID NOs: 30, 33 and 35 respectively;
(32) the VH CDRs 1, 2, 3 amino acid sequences are set forth in SEQ ID NOs: 21, 27 and 29 respectively, and the selected VL CDRs 1, 2, 3 amino acid sequences are set forth in SEQ ID NOs: 30, 33 and 35 respectively;
(33) the VH CDRs 1, 2, 3 amino acid sequences are set forth in SEQ ID NOs: 21, 28 and 29 respectively, and the selected VL CDRs 1, 2, 3 amino acid sequences are set forth in SEQ ID NOs: 30, 33 and 35 respectively;
(34) the VH CDRs 1, 2, 3 amino acid sequences are set forth in SEQ ID NOs: 20, 22 and 29 respectively, and the selected VL CDRs 1, 2, 3 amino acid sequences are set forth in SEQ ID NOs: 30, 33 and 36 respectively;
(35) the VH CDRs 1, 2, 3 amino acid sequences are set forth in SEQ ID NOs: 21, 24 and 29 respectively, and the selected VL CDRs 1, 2, 3 amino acid sequences are set forth in SEQ ID NOs: 30, 33 and 36 respectively;
(36) the VH CDRs 1, 2, 3 amino acid sequences are set forth in SEQ ID NOs: 21, 25 and 29 respectively, and the selected VL CDRs 1, 2, 3 amino acid sequences are set forth in SEQ ID NOs: 30, 33 and 36 respectively;
(37) the VH CDRs 1, 2, 3 amino acid sequences are set forth in SEQ ID NOs: 21, 26 and 29 respectively, and the selected VL CDRs 1, 2, 3 amino acid sequences are set forth in SEQ ID NOs: 30, 33 and 36 respectively;
(38) the VH CDRs 1, 2, 3 amino acid sequences are set forth in SEQ ID NOs: 21, 27 and 29 respectively, and the selected VL CDRs 1, 2, 3 amino acid sequences are set forth in SEQ ID NOs: 30, 33 and 36 respectively;
(39) the VH CDRs 1, 2, 3 amino acid sequences are set forth in SEQ ID NOs: 21, 28 and 29 respectively, and the selected VL CDRs 1, 2, 3 amino acid sequences are set forth in SEQ ID NOs: 30, 33 and 36 respectively;
(40) the VH CDRs 1, 2, 3 amino acid sequences are set forth in SEQ ID NOs: 20, 22 and 29 respectively, and the selected VL CDRs 1, 2, 3 amino acid sequences are set forth in SEQ ID NOs: 30, 33 and 37 respectively;
(41) the VH CDRs 1, 2, 3 amino acid sequences are set forth in SEQ ID NOs: 21, 24 and 29 respectively, and the selected VL CDRs 1, 2, 3 amino acid sequences are set forth in SEQ ID NOs: 30, 33 and 37 respectively;
(42) the VH CDRs 1, 2, 3 amino acid sequences are set forth in SEQ ID NOs: 21, 25 and 29 respectively, and the selected VL CDRs 1, 2, 3 amino acid sequences are set forth in SEQ ID NOs: 30, 33 and 37 respectively;
(43) the VH CDRs 1, 2, 3 amino acid sequences are set forth in SEQ ID NOs: 21, 26 and 29 respectively, and the selected VL CDRs 1, 2, 3 amino acid sequences are set forth in SEQ ID NOs: 30, 33 and 37 respectively;
(44) the VH CDRs 1, 2, 3 amino acid sequences are set forth in SEQ ID NOs: 21, 27 and 29 respectively, and the selected VL CDRs 1, 2, 3 amino acid sequences are set forth in SEQ ID NOs: 30, 33 and 37 respectively;
(45) the VH CDRs 1, 2, 3 amino acid sequences are set forth in SEQ ID NOs: 21, 28 and 29 respectively, and the selected VL CDRs 1, 2, 3 amino acid sequences are set forth in SEQ ID NOs: 30, 33 and 37 respectively;
(46) the VH CDRs 1, 2, 3 amino acid sequences are set forth in SEQ ID NOs: 20, 22 and 29 respectively, and the selected VL CDRs 1, 2, 3 amino acid sequences are set forth in SEQ ID NOs: 30, 32 and 35 respectively;
(47) the VH CDRs 1, 2, 3 amino acid sequences are set forth in SEQ ID NOs: 21, 24 and 29 respectively, and the selected VL CDRs 1, 2, 3 amino acid sequences are set forth in SEQ ID NOs: 30, 32 and 35 respectively;
(48) the VH CDRs 1, 2, 3 amino acid sequences are set forth in SEQ ID NOs: 21, 25 and 29 respectively, and the selected VL CDRs 1, 2, 3 amino acid sequences are set forth in SEQ ID NOs: 30, 32 and 35 respectively;
(49) the VH CDRs 1, 2, 3 amino acid sequences are set forth in SEQ ID NOs: 21, 26 and 29 respectively, and the selected VL CDRs 1, 2, 3 amino acid sequences are set forth in SEQ ID NOs: 30, 32 and 35 respectively;
(50) the VH CDRs 1, 2, 3 amino acid sequences are set forth in SEQ ID NOs: 21, 27 and 29 respectively, and the selected VL CDRs 1, 2, 3 amino acid sequences are set forth in SEQ ID NOs: 30, 32 and 35 respectively;
(51) the VH CDRs 1, 2, 3 amino acid sequences are set forth in SEQ ID NOs: 21, 28 and 29 respectively, and the selected VL CDRs 1, 2, 3 amino acid sequences are set forth in SEQ ID NOs: 30, 32 and 35 respectively.

2.-5. (canceled)

6. The antibody or antigen-binding fragment thereof of claim 1, wherein the heavy chain variable region (VH) comprising an amino acid sequence that is at least 80%, 85%, 90%, 95% or 100% identical to a selected VH sequence, and the light chain variable region (VL) comprising an amino acid sequence that is at least 80%, 85%, 90%, 95% or 100% identical to a selected VL sequence, wherein the combination of the selected VH sequence and VL sequence is one of the following:
(1) the selected VH sequence is SEQ ID NO: 5, and the selected VL sequence is SEQ ID NO: 4;
(2) the selected VH sequence is SEQ ID NO: 6, and the selected VL sequence is SEQ ID NO: 4;
(3) the selected VH sequence is SEQ ID NO: 7, and the selected VL sequence is SEQ ID NO: 4;
(4) the selected VH sequence is SEQ ID NO: 8, and the selected VL sequence is SEQ ID NO: 4;
(5) the selected VH sequence is SEQ ID NO: 11, and the selected VL sequence is SEQ ID NO: 4;
(6) the selected VH sequence is SEQ ID NO: 12, and the selected VL sequence is SEQ ID NO: 4;
(7) the selected VH sequence is SEQ ID NO: 13, and the selected VL sequence is SEQ ID NO: 4;
(8) the selected VH sequence is SEQ ID NO: 14, and the selected VL sequence is SEQ ID NO: 4;
(9) the selected VH sequence is SEQ ID NO: 15, and the selected VL sequence is SEQ ID NO: 4;
(10) the selected VH sequence is SEQ ID NO: 5, and the selected VL sequence is SEQ ID NO: 9;
(11) the selected VH sequence is SEQ ID NO: 6, and the selected VL sequence is SEQ ID NO: 9;
(12) the selected VH sequence is SEQ ID NO: 7, and the selected VL sequence is SEQ ID NO: 9;
(13) the selected VH sequence is SEQ ID NO: 8, and the selected VL sequence is SEQ ID NO: 9;
(14) the selected VH sequence is SEQ ID NO: 11, and the selected VL sequence is SEQ ID NO: 9;
(15) the selected VH sequence is SEQ ID NO: 12, and the selected VL sequence is SEQ ID NO: 9;
(16) the selected VH sequence is SEQ ID NO: 13, and the selected VL sequence is SEQ ID NO: 9;
(17) the selected VH sequence is SEQ ID NO: 14, and the selected VL sequence is SEQ ID NO: 9;
(18) the selected VH sequence is SEQ ID NO: 15, and the selected VL sequence is SEQ ID NO: 9;
(19) the selected VH sequence is SEQ ID NO: 8, and the selected VL sequence is SEQ ID NO: 16;
(20) the selected VH sequence is SEQ ID NO: 8, and the selected VL sequence is SEQ ID NO: 17;
(21) the selected VH sequence is SEQ ID NO: 8, and the selected VL sequence is SEQ ID NO: 18;
(22) the selected VH sequence is SEQ ID NO: 8, and the selected VL sequence is SEQ ID NO: 19;
(23) the selected VH sequence is SEQ ID NO: 8, and the selected VL sequence is SEQ ID NO: 137;
(24) the selected VH sequence is SEQ ID NO: 5, and the selected VL sequence is SEQ ID NO: 16;
(25) the selected VH sequence is SEQ ID NO: 5, and the selected VL sequence is SEQ ID NO: 17;
(26) the selected VH sequence is SEQ ID NO: 5, and the selected VL sequence is SEQ ID NO: 18;
(27) the selected VH sequence is SEQ ID NO: 5, and the selected VL sequence is SEQ ID NO: 19;
(28) the selected VH sequence is SEQ ID NO: 5, and the selected VL sequence is SEQ ID NO: 137;
(29) the selected VH sequence is SEQ ID NO: 11, and the selected VL sequence is SEQ ID NO: 16;
(30) the selected VH sequence is SEQ ID NO: 11, and the selected VL sequence is SEQ ID NO: 17;
(31) the selected VH sequence is SEQ ID NO: 11, and the selected VL sequence is SEQ ID NO: 18;
(32) the selected VH sequence is SEQ ID NO: 11, and the selected VL sequence is SEQ ID NO: 19;
(33) the selected VH sequence is SEQ ID NO: 11, and the selected VL sequence is SEQ ID NO: 137;
(34) the selected VH sequence is SEQ ID NO: 12, and the selected VL sequence is SEQ ID NO: 16;
(35) the selected VH sequence is SEQ ID NO: 12, and the selected VL sequence is SEQ ID NO: 17;
(36) the selected VH sequence is SEQ ID NO: 12, and the selected VL sequence is SEQ ID NO: 18;
(37) the selected VH sequence is SEQ ID NO: 12, and the selected VL sequence is SEQ ID NO: 19;
(38) the selected VH sequence is SEQ ID NO: 12, and the selected VL sequence is SEQ ID NO: 137;
(39) the selected VH sequence is SEQ ID NO: 13, and the selected VL sequence is SEQ ID NO: 16;
(40) the selected VH sequence is SEQ ID NO: 13, and the selected VL sequence is SEQ ID NO: 17;
(41) the selected VH sequence is SEQ ID NO: 13, and the selected VL sequence is SEQ ID NO: 18;
(42) the selected VH sequence is SEQ ID NO: 13, and the selected VL sequence is SEQ ID NO: 19;
(43) the selected VH sequence is SEQ ID NO: 13, and the selected VL sequence is SEQ ID NO: 137;
(44) the selected VH sequence is SEQ ID NO: 14, and the selected VL sequence is SEQ ID NO: 16;
(45) the selected VH sequence is SEQ ID NO: 14, and the selected VL sequence is SEQ ID NO: 17;
(46) the selected VH sequence is SEQ ID NO: 14, and the selected VL sequence is SEQ ID NO: 18;
(47) the selected VH sequence is SEQ ID NO: 14, and the selected VL sequence is SEQ ID NO: 19;
(48) the selected VH sequence is SEQ ID NO: 14, and the selected VL sequence is SEQ ID NO: 137;
(49) the selected VH sequence is SEQ ID NO: 15, and the selected VL sequence is SEQ ID NO: 16;
(50) the selected VH sequence is SEQ ID NO: 15, and the selected VL sequence is SEQ ID NO: 17;
(51) the selected VH sequence is SEQ ID NO: 15, and the selected VL sequence is SEQ ID NO: 18;
(52) the selected VH sequence is SEQ ID NO: 15, and the selected VL sequence is SEQ ID NO: 19;
(53) the selected VH sequence is SEQ ID NO: 15, and the selected VL sequence is SEQ ID NO: 137.

7. (canceled)

8. The antibody or antigen-binding fragment thereof of claim 1, wherein the antibody or antigen-binding fragment comprises a single-chain variable fragment (scFv), wherein the scFv comprises an amino acid sequence selected from SEQ ID NOs: 39-46 and 62-96.

9.-15. (canceled)

16. A polynucleotide encoding the antibody or antigen-binding fragment thereof of claim 1.

17.-19. (canceled)

20. An engineered receptor comprising the antigen-binding fragment thereof of claim 1.

21. The engineered receptor of claim 20, wherein the engineered receptor further comprises a transmembrane domain, and an intracellular signaling domain.

22. The engineered receptor of claim 20, wherein the engineered receptor is a chimeric antigen receptor (“CAR”) or a chimeric T cell receptor.

23. (canceled)

24. The engineered receptor of claim 21, wherein the transmembrane domain comprises a transmembrane domain of CD4, CD8, and/or CD28, or a portion thereof.

25. (canceled)

26. The engineered receptor of claim 21, wherein the intracellular signaling domain is or comprises a functional signaling domain of CD3 zeta.

27. The engineered receptor of claim 21, wherein the intracellular signaling domain further comprises a costimulatory signaling domain.

28. The engineered receptor of claim 27, wherein the costimulatory signaling domain comprises a functional signaling domain from a protein selected from the group consisting of a MHC class I molecule, a TNF receptor protein, an Immunoglobulin-like protein, a cytokine receptor, an integrin, a signaling lymphocytic activation molecule (SLAM protein), an activating NK cell receptor, BTLA, a Toll ligand receptor, OX40, CD2, CD7, CD27, CD28, CD30, CD40, CDS, ICAM-1, LFA-1, CD11a/CD18, 4-1BB (CD137), B7-H3, CDS, ICAM-1, ICOS (CD278), GITR, BAFFR, LIGHT, HVEM (LIGHTR), KIRDS2, SLAMF7, NKp80 (KLRF1), NKp44, NKp30, NKp46, CD19, CD4, CD8alpha, CD8beta, IL2R beta, IL2R gamma, IL7R alpha, ITGA4, VLA1, CD49a, ITGA4, IA4, CD49D, ITGA6, VLA-6, CD49f, ITGAD, CD11d, ITGAE, CD103, ITGAL, CD11a, LFA-1, ITGAM, CD 11b, ITGAX, CD 11c, ITGB1, CD29, ITGB2, CD18, LFA-1, ITGB7, NKG2D, NKG2C, TNFR2, TRANCE/RANKL, DNAM1 (CD226), SLAMF4 (CD244, 2B4), CD84, CD96 (Tactile), CEACAMI, CRTAM, Ly9 (CD229), CD160 (BY55), PSGL1, CD100 (SEMA4D), CD69, SLAMF6 (NTB-A, Lyl08), SLAM (SLAMF1, CD150, IPO-3), BLAME (SLAMF8), SELPLG (CD162), LTBR, LAT, GADS, SLP-76, PAG/Cbp, CD19a, and a CD83 ligand.

29.-31. (canceled)

32. The engineered receptor of claim 20, wherein the engineered receptor comprises an amino acid sequence set forth in any one of SEQ ID NOs: 54-61 and 97-131, or an amino acid sequence that is at least 90%, 91%, 92%, 93%, 94%, 95%, 96%, 97%, 98%, 99% or 100% identical to any one of SEQ ID NOs: 54-61 and 97-131.

33.-40. (canceled)

41. A polynucleotide encoding the engineered receptor of claim 20.

42. A vector comprising the polynucleotide of claim 41.

43. (canceled)

44. An engineered cell expressing the engineered receptor of claim 20.

45. The engineered cell of claim 44, wherein the engineered cell is an immune cell.

46. The engineered cell of claim 45, wherein the immune cell is selected from the group consisting of an NK cell, a cytotoxic T cell, a helper T cell, a natural killer T (NK-T) cell, a αβ T cell, and a γδ T cell.

47. (canceled)

48. (canceled)

49. A pharmaceutical composition comprising the engineered cell of claim 20 and a pharmaceutically acceptable carrier.

50. (canceled)

51. A method of treating cancer in a subject, comprising administering an effective amount of the pharmaceutical composition of claim 49 to the subject.

52. The method of claim 51, wherein the cancer is liver cancer.
